(19) United States
(12) Patent Application Publication
(10) Pub. No.: US 2025/0170739 A1
(43) Pub. Date: May 29, 2025

(54) PERSONAL CARE PRODUCT

(71) Applicant: TRISA HOLDING AG, Triengen (CH)

(72) Inventors: Armin BÄRTSCHI, Winznau (CH); Herbert Fischer, Reitnau (CH); Martin Zwimpfer, Luzern (CH)

(73) Assignee: TRISA HOLDING AG, Triengen (CH)

(21) Appl. No.: 19/042,170

(22) Filed: Jan. 31, 2025

Related U.S. Application Data
(62) Division of application No. 18/842,502, filed on Dec. 4, 2024.

(30) Foreign Application Priority Data
Mar. 1, 2022 (EP) 22159453.4

(51) Int. Cl. B26B 21/52

(52) U.S. Cl. CPC B26B 21/522; B26B 21/521

(57) ABSTRACT

A body care product, in particular a toothbrush, a razor or an application brush, includes at least one application unit, further includes a handle unit comprising at least one material handle body which is at least largely made of a paper material, and includes at least one connection unit which connects the application unit with the handle unit, wherein the connection unit is configured at least partially for a form-fitting connection to the material handle body of the handle unit.

### CROSS-REFERENCE TO RELATED APPLICATIONS

This application is a Divisional application of U.S. patent application Ser. No. 18/842,502, filed Aug. 29, 2024, which is a U.S. national stage application of international patent application PCT/EP2023/055207, filed on Mar. 1, 2023, which is based on and claims priority to European patent application EP 22159453.4, filed on Mar. 1, 2022, the contents of which are incorporated herein by reference.

### PRIOR ART

The invention concerns a body care product.

It is a body care product, in particular a toothbrush, a flosser, a razor, an application brush or an applicator, with at least one application unit, with a handle unit comprising at least one material handle body which is at least largely made of a paper material, and with at least one connection unit which connects the application unit with the handle unit.

Oral hygiene means are already known from DE 20 2020 000 613 U1, WO 2014/169398 A1, WO 2021/081438 A1 and DE 10 2020 100 106 A1.

The objective of the invention is in particular to provide a generic device having improved properties with regard to manufacturability, comfort and ecology. The objective is achieved according to the invention.

### Advantages of the Invention

The invention is based on a body care product, in particular a toothbrush, a flosser, a razor, an application brush or an applicator, with at least one application unit, with a handle unit comprising at least one material handle body which is at least largely made of a paper material, and with at least one connection unit which connects the application unit with the handle unit. An applicator may also mean a mascara applicator.

It is proposed that the connection unit is configured at least partially for a form-fitting connection to the material handle body of the handle unit. Additionally or alternatively to the form-fitting connection, a force-fitting and/or material-bonding connection may also be provided. It is in particular conceivable that the connection unit is additionally or alternatively at least partially glued, welded, cast or material-bonded with the material handle body of the handle unit. It would also be conceivable that a connection is created by means of injection-molding and/or overmolding or by means of clamping, snap-action or riveting.

“At least largely” is in particular to mean that at least a main constituent of a material of the material handle body of the handle unit and/or of the application unit is a paper material. Preferably, at least 50 wt. % (weight percent), preferably at least 70 wt. % and particularly preferably at least 90 wt. % of the material handle body of the handle unit are made of a paper material. Particularly preferably the material handle body of the handle unit consists completely of a paper material. Furthermore, in this context a “paper material” is in particular to mean a—preferably planar—material which essentially consists of fibers, preferably of plant origin. The material is preferably formed by dehydration of a fiber suspension on a sieve. The resulting fiber fleece is then in particular compacted and dried. Preferably, the material is produced from pulp, groundwood and/or waste paper. It is conceivable to replace the paper by another sustainable material alternative, such as wood or bamboo. The alternatives are also preferably provided in planar fashion at least as a raw material or as a semi-finished product.

Preferably the paper material in particular consists of fibers, wherein there are in particular various processing possibilities. The paper material may also have been subjected to treatment. The paper material may be coated, such as in particular varnished, for example with a varnish for improving the connection during bonding and/or for improving the water resistance, and/or may be impregnated. The paper material may in particular be present both in flat form, in block form or in liquid to cardboard form and/or consistency, wherein starting from the form various processing operations are possible. In a flat form the paper material may be rolled, layered, pressed, laminated, creased, embossed, folded, punched and/or shagreened, that is to say in particular roughened or grained. For a connection, the paper material may be glued, wherein possibly preparation steps such as shagreening or the like are necessary; it may be welded, such as in particular by means of ultrasonic welding; it may be sealed (hot or cold) or laminated, etc. For a connection, special layers may be necessary on the paper material in order to create and/or improve the connection. Connections may be produced with a paper material or with other substances and/or materials mentioned in this document. In a block form the paper material may be processed mechanically, for example milled, drilled or punched. In a liquid form the paper material may be cast or injection-molded, such as in particular by paper injection-molding or paper casting and/or fiber casting. Herein the paper material is molded in a cavity, in particular analogously to an injection-molding process. Subsequently the cast part is hardened and/or dried. Preferably, in paper injection-molding or paper casting and/or fiber casting, no post-processing will be necessary on the body of paper material, but post-processing is possible, for example mechanical post-processing, for example by milling, drilling or punching. In paper injection-molding, in particular injection-molding onto other parts and/or materials is also conceivable, for example plastic overmolded with paper. Alternatively, a plastic part may also be injection-molded to a paper material by injection-molding of the plasticized plastic (e. g. a thermoplastic) to the paper material in a form-fitting and/or force-fitting manner. All in all, the product structure of the body care product may be of different types, such as in particular completely of a paper material or with a hybrid technology, e. g. of paper material combined with plastic, in particular for example with an exchangeable head and/or with a handle unit of paper and an application unit of plastic. As has been mentioned, further material combinations with paper may be plastic and/or metal and/or organic materials such as wood, bamboo and/or inorganic materials such as stone, glass etc. It should be noted that—for the sake of simplicity—in this document paper is described as a preferred material for the handle unit; it is however actually within the meaning of the invention to replace paper by a material that preferably consists of essentially sustainable components, such as wood or bamboo. The material for the handle unit is provided at least as a raw material and/or as a semi-finished product, preferably in a flat form.

The invention in particular describes that the paper material is preferably a planarly prepared material. For this reason, as has been described, the paper material may be used like a synonym for wood, veneer or also bamboo materials, which preferably are planarly prepared and/or processed in a manner corresponding to the paper. The described implementations with paper material may thus be embodied in a synonym-wise manner to the above-mentioned alternative materials.

The different aspects of the invention are described in connection with bodies that are preferably implemented of flat raw materials. However, in principle the aspects may also be applied to more voluminous bodies, for example cylindrical bodies (rods) or other volumes in 3D form. This is used for example in connection with the described paper injection-molding or paper casting and/or fiber casting methods.

The implementation of the body care product according to the invention allows providing advantageous properties with regard to ergonomics and to manufacturability of the body care product. It is in particular possible to provide an advantageously ecological body care product, respectively a body care product made to a significant extent of sustainable materials. In particular, an advantageously simple separation and/or disposal of the body care product are/is achievable.

The body care product is in particular realized as a razor, in particular a manual razor, as a brush, in particular an application brush for cosmetic applications such as e. g. for hair dyeing, or as an applicator for medical applications such as e. g. applicators in the dental field like for example application brushes for auxiliary agents for fillings, crowns or the like, or as an applicator for cosmetic applications such as e. g. an applicator for mascara, and/or as an oral hygiene means. The razor may be realized, for example, as a disposable razor or as a reusable razor with an exchangeable blade. An “oral hygiene means” is in particular to mean a toothbrush and/or a tongue cleaner or a flosser. Advantageously the oral hygiene means is realized as a toothbrush, in particular as a manual toothbrush. Herein, in a realization as a toothbrush, the oral hygiene means may in particular be a disposable (single-use) toothbrush, a reusable (multiple-use) toothbrush or also an exchangeable-head toothbrush. However, it is also possible that the oral hygiene means is generally realized as a brush product. A “body care product” is in particular to mean an oral hygiene brush and/or a cosmetic brush and/or a hair brush and/or a household brush. For example, manual toothbrushes such as reusable toothbrushes, exchangeable-head toothbrushes, plug-on brushes for electric toothbrushes, disposable toothbrushes or single-tuft brushes or tongue cleaners or flossers are conceivable as oral hygiene brushes. Furthermore, as body care products, in particular cosmetic brushes, for example mascara brushes, nail polish brushes, face brushes, applicators, massaging devices, make-up brushes, shaving brushes and/or wet shavers or other body care products are conceivable.

Generally, medical applicators constructed in a similar way as the body care products may also be realized with the present invention, for example applicators in the dental field like for example application brushes for auxiliary agents for fillings, crowns or the like, or an applicator for cosmetic applications, for example an applicator for mascara.

In an implementation as a toothbrush, it is in particular conceivable that the application unit is composed of a base body and a bristle platelet which is studded with bristles and/or alternative cleaning elements. The application unit may furthermore consist of a base body which is overmolded and/or studded with bristles, or the application unit may be injection-molded directly, respectively integrally, without a base body. Preferably the body care product consists of an application unit with bristles, the connection unit and the handle unit, wherein the handle unit in particular has a neck portion. All injection-molded sub-elements may consist of at least one hard component and/or one or several soft component/s, and/or one or several materials for injection-molded bristles.

The body care product in particular has a longitudinal axis which is advantageously arranged at least substantially parallel to a main extension direction of the body care product. The longitudinal axis preferably runs at least section-wise within the body care product and in particular through the center of gravity of the body care product.

In particular, the longitudinal axis of the body care product is a central axis of the body care product and/or a central axis of the handle unit. By a “central axis” of an object is here in particular an imaginary axis to be understood which runs within the object parallel to a main extension direction of the object and intersects with the object in at most two points. “At least substantially parallel” is here in particular to mean an orientation of a direction relative to a reference direction, in particular in a plane, wherein the direction has a deviation from the reference direction that is in particular smaller than 8°, advantageously smaller than 5° and particularly advantageously smaller than 2°. By a “main extension direction” of an object is here in particular a direction to be understood which runs parallel to a longest edge of a smallest imaginary cuboid just still completely enclosing the object. A “main extension” of an object is in particular to mean, in this context, an extension of a longest edge of a smallest imaginary cuboid just still completely enclosing the object.

Advantageously, the application unit comprises at least one cleaning zone, which is configured for a tooth cleaning application, in particular in an oral cavity of the user. Preferably the cleaning zone comprises at least one cleaning unit. The cleaning unit may comprise a brush head, advantageously a toothbrush head, preferably with a plurality of bristles and/or bristle bundles and/or injection-molded cleaning elements, respectively injection-molded bristles and/or soft-elastic cleaning elements. Alternatively or additionally, the cleaning unit may also be realized as a single tuft (i. e. as a single large bristle bundle), as an interdental-space cleaning element (e. g. dental floss, toothpick, stick etc.) or the like. Moreover, the application unit advantageously comprises at least one base body, which is preferably connected, in particular directly and/or integrally, with the cleaning zone. Alternatively or additionally, the application unit comprises at least one application region which is configured, for example, for storing and dispensing a dye, in particular a hair dye. Alternatively or additionally, the application unit comprises at least one shaving region with at least one blade. Alternatively or additionally, the application unit comprises at least one application region which is configured, for example, for storing and dispensing a medium, in particular an auxiliary agent for fillings, crowns or the like, in particular a dental adhesive, a dental primer or the like. This application region is realized as an applicator head, the shape of the applicator head preferably being spherical or ellipsoidal. The surface of the applicator head is designed with elements supporting the application, respectively supporting the storage and/or dispensing of a medium. Herein, for example, recesses may be formed in the surface which make this possible, or the applicator head may be provided with a flocking which makes this possible. The recesses may be realized around the applicator head or only partially on certain sub-surfaces. The recesses may be realized as individual insulated elements—for example in the form of individual strips, or the recess may be realized as a continuous shape extending over the entire body—for example in the form of a spiral.

The design of such an applicator head may be realized by injection molding. The applicator head may be injection-molded onto a handle body, the applicator head being injection-molded around the handle body, and the connection between the applicator head and the handle body being implemented in this way.

“Integrally” is in particular to mean at least materially-bonded, for example by a connection process such as a welding process, a sealing process, an adhesive process, an injection-molding process and/or another process deemed expedient by someone skilled in the art, and/or advantageously formed in one piece, such as for example by a production from a casting and/or by a production in a one-component or multi-component injection-molding method, and advantageously from a single blank. “Configured” is in particular to mean specifically designed and/or equipped. By an object being configured for a specific function is in particular to be understood that the object fulfils and/or carries out this specific function in at least one application state and/or operation state.

Preferably the handle unit comprises at least one handle element, which is advantageously configured to be held with one hand. It is conceivable that the handle element is waisted at least region-wise and/or is formed with a concave surface region and/or with a convex surface region. The handle element preferably at least region-wise has a cross-section that forms a U-profile. This advantageously allows a secure hold and optimizes ergonomics. Particularly preferably the handle element is realized so as to be elongate, wherein advantageously a longitudinal axis of the handle element corresponds to the longitudinal axis of the body care product. The handle element is at least substantially, in particular completely, made of at least one paper material. Alternatively or additionally, the handle element and/or the handle unit may be made at least partially of a hard component and/or of one or several soft components and/or of material for injection-molded bristles. Preferably the material handle body is at least largely, in particular completely, made of at least one paper material. In particular, the handle element advantageously comprises at least one thumb gripping region and/or at least one hand gripping region. Advantageously the thumb gripping region is arranged on the front side of the body care product, and in particular on a front side of the handle element. It is conceivable that the thumb gripping region and/or the hand gripping region comprise/comprises at least one element and/or a surface structuring made of at least one paper material, of at least one soft component and/or of at least one hard component.

The synthetic materials consisting of hard and/or soft components, which are used for the production and are processed by injection molding, may in particular be classified as follows:
Conventional materials: materials, essentially new materials, which are largely petroleum-based.
Sustainable materials: as listed below and described later, preferably biobased, degradable and/or recycled.
   Biobased materials: material produced, in particular by more than 60%, preferably by more than 80% and particularly preferably by 100% from sustainable raw materials. A further possible additional property of biobased materials is in particular that the biobased materials are biodegradable. The materials are preferably not based on food products, such as in particular corn etc.
   Biodegradable materials: material which is biodegradable according to current standards. This in particular includes compostability (industrial or non-industrial). Herein materials made of sustainable raw materials may have this property.
   Recycled materials: materials resulting from a recycling process, such as for example post-consumer recycled materials, post-industrial recycled materials, ocean waste plastic or social plastic.

The materials used may be recyclable materials. For recyclable materials there is advantageously a possibility of recycling after use.

Within the scope of this disclosure, almost any hard components and soft components are possible, which will be suitably and purposefully combined and/or selected by someone skilled in the art. Suitable hard components are for example styrene polymers such as styrene acrylonitrile (SAN), polystyrene (PS), acrylonitrile butadiene styrene (ABS), styrene methyl methacrylate (SMMA), styrene butadiene (SB) or the like. Furthermore, a hard component may comprise polyolefins such as polypropylene (PP), polyethylene (PE) or the like, in particular also in the form of high-density polyethylene (HDPE) or low-density polyethylene (LDPE). Also suitable are polyesters, such as for example polyethylene terephthalate (PET), in particular in the form of acid-modified polyethylene terephthalate (PETA) or glycol-modified polyethylene terephthalate (PETG), polybutylene terephthalate (PBT), acid-modified polycyclohexylene dimethylene terephthalate (PCT-A), glycol-modified polycyclohexylene dimethylene terephthalate (PCT-G) or the like. Using cellulose derivatives, such as for example cellulose acetate (CA), cellulose acetobutyrate (CAB), cellulose propionate (CP), cellulose acetate phthalate (CAP), cellulose butyrate (CB) or the like, is also conceivable. Furthermore, a hard component may comprise, for example, polyamides (PA) such as PA 6.6, PA 6.10, PA 6.12 or the like, polymethyl methacrylate (PMMA), polycarbonate (PC), polyoxymethylene (POM), polyvinyl chloride (PVC), polyurethane (PUR), polyamide (PA) or the like. In particular polyethylene (PE) and polyurethane (PU) may be used as a hard component and/or as a soft component. In particular, a hard component has a modulus of elasticity of at least 1,000 N/mm², and advantageously of at least 1,300 N/mm², and/or of at most 2,400 N/mm² and advantageously of at most 1,800 N/mm². Polypropylene (PP) is preferably used as a hard component. At least certain materials mentioned as a hard component may be sustainable materials. In particular, materials with a cellulose content are at least partially biobased.

Advantageously hard components are used for stable and/or structure-bearing elements, in particular in the handle element and/or in a connection element of the application unit or the like. Advantageously it is conceivable that a hard component used and a soft component used have different colors, such that surface structures, inscriptions, motifs and the like can be realized by a suitable design of base body and soft element.

Suitable soft components are, for example, thermoplastic styrene elastomers (TPE-S), such as for instance a styrene-ethylene-butylene-styrene copolymer (SEBS), a styrene-butadiene-styrene copolymer (SBS) or the like. Beyond this, it is conceivable to use thermoplastic polyurethane elastomers (TPE-U), thermoplastic polyamide elastomers (TPE-A), thermoplastic polyolefin elastomers (TPE-O), thermoplastic polyester elastomers (TPE-E) or the like. Furthermore, a soft component may comprise, for example, at least one silicone. Advantageously a soft component has a Shore A hardness of at most 90, advantageously of at most 50 and particularly advantageously of at most 30. Preferably at least one soft component forms at least a material bond with at least one hard component, in particular in at least one two-component and/or multi-component injection molding, advantageously at least by overmolding and/or injection-molding-around. The materials mentioned for the soft component may be sustainable materials. Hard component, soft component or material for injection-molded bristles do not form a material bond with the paper material.

In an implementation as a toothbrush, the body care product in particular has a front side and a rear side, which are in particular arranged facing away from each other. Preferably the cleaning zone of the application unit is arranged on the front side of the body care product. The front side is in particular a side of the body care product that is visible in a viewing direction perpendicular to the longitudinal axis of the body care product and perpendicular to the width axis of the body care product. In particular, the side of the brush on which the thumb is placed is referred to as the front side of the body care product. The front side is normally also the side to which the bristle field is directed. The rear side advantageously corresponds to a side of the body care product that is visible in an opposed viewing direction. The side of the toothbrush opposed to the bristle field is referred to as the rear side of the body care product. In particular, a side that lies to the left when looking perpendicularly at the front side of the body care product, with the cleaning zone situated at the top, is referred to as the lefthand side of the body care product. In particular, a side that lies to the right when looking perpendicularly at the front side of the body care product is referred to as the righthand side of the body care product. In particular, an end of the body care product at which the cleaning zone is arranged is referred to as the top side. In particular, an end of the body care product situated opposite the top side and closest to the handle region is referred to as the bottom side.

In this context, a “connection unit” is in particular to mean a unit which is configured to provide a fix and/or releasable connection between two units, in particular components. Preferably, the connection unit connects the application unit to the handle unit. Preferentially the application unit and the handle unit are connected to one another at least for use. Preferably the connection unit may be realized as a separate unit and may at least partially be fixedly connected with the handle unit and/or the application unit. That at least one first element is “connected” with at least one further element is in particular to mean that the first element is advantageously connected with the further element via at least one force-fitting connection and/or at least one form-fitting connection, for example via a riveting and/or via a latching connection and/or via a tongue-and-groove connection and/or via a rivet connection and/or via a clamping connection and/or via a further connection deemed expedient by someone skilled in the art, and/or is materially-bonded with the further element, for example by a welding process and/or a sealing process and/or an adhesive process, an injection-molding, respectively overmolding, process and/or a deformation process and/or another process deemed expedient by someone skilled in the art. In this context, a “form-fitting connection” is in particular to mean a connection in which adjoining surfaces, of components connected with one another in a form-fitting manner, exert a holding force onto one another that acts in the normal direction of the surfaces. In particular, the components are in geometrical engagement with one another. Preferably, a form-fitting connection is in particular provided between the application unit and the handle unit, for example by geometries that are adapted to one another. Thus recesses, ridges and/or form-fitting elements may in particular be provided on the application unit, the handle unit and/or the connection unit. In particular, the form-fitting connection may also serve merely for an orientation of the connection possibilities in order to enable a simple connection during assembly or in order to absorb harmful transverse forces, for example in the case of an adhesive connection. The orientation of the connection possibilities may in particular take place around the handle unit, for example in the case of a brush on the front side, on the rear side or on the short sides. If there are long and short sides of a body care product, an orientation of the connection possibilities in particular takes place on a long side.

The connection unit in particular has the function of connecting the application unit or an interface of the application unit to the handle unit. The connection unit in particular has several functions. In particular, the connection unit is configured for connecting the handle unit and for holding the application unit. The connection unit in particular forms an interface to the handle unit. Furthermore, the connection unit may in particular comprise an exchanging system by means of which the application unit can be exchanged. The connection unit may also support the application unit movably. In addition, the connection unit may for example have a holding geometry for gripping the body care product, for example during use. The connection unit may have means facilitating a separation from the handle unit and/or from the application unit (e. g. for appropriate disposal). Thus, in particular a combination of various of the mentioned functions is conceivable, such as for example an inside of an insertion geometry or of a support geometry and an outside of a holding element.

It is further proposed that the connection unit comprises at least one form-fitting element, which is configured to engage into a recess of the application unit and/or of the handle unit. Preferably the form-fitting element is in particular configured to provide a geometrical engagement, wherein the form-fitting element geometrically engages into the recess, such that in a plane perpendicular to a central axis of the recess the form-fitting element is completely encompassed by a component that delimits the recess. Preferably the form-fitting element is delimited directly by a delimiting surface of the application unit and/or of the handle unit which delimits the recess. Preferably the form-fitting element may be realized, for example, as a latching pin, a rivet, a spring or the like. Preferably the form-fitting element is configured to engage into a recess of the application unit and/or of the handle unit and to create in this recess, for example by an undercut and/or by engaging on the rear, a form-fitting connection that extends beyond a form-fitting connection transversely to an insertion direction into the recess. This in particular allows providing an advantageous connection unit. It is in particular possible to provide an advantageously easily producible and stable connection. It is preferably possible to create a connection that, in the separation from the handle unit and/or the application unit, makes the handle unit and/or the application unit unusable, respectively not reconnectable.

Furthermore, it is proposed that the material handle body delimits at least one pass-through recess, which is configured to receive the at least one form-fitting element of the connection unit. This in particular advantageously allows providing a form-fitting recess for the form-fitting element. It is moreover advantageously possible to introduce a form-fitting recess into the material base body in an advantageously simple manner. The pass-through recess is preferably realized so as to correspond to the form-fitting element of the connection unit. The connection unit is preferably configured for a form-fitting connection to the handle unit. The pass-through recess is realized, for example, as a cylindrical recess, for example a circular-cylindrical recess. The at least one pass-through recess extends completely through the material handle body. The at least one pass-through recess preferably extends through the material handle body perpendicular to a main extension plane of the material handle body. A “main extension plane” of an object is in particular to mean a plane which is parallel to a largest side surface of a smallest imaginary cuboid just still completely enclosing the object, and which extends through the center point of the cuboid. The at least one pass-through recess in particular forms a counter-contour for the form-fitting element of the connection unit. Alternatively or additionally, lateral notches may also be formed on the handle body for the same purpose.

Beyond this it is proposed that the form-fitting element is realized as a latching element. A “latching element” is in particular to mean, in this context, a spring-elastic element for producing a latching connection, which is configured to be elastically deflected during assembly. The latching element is preferably configured to be elastically deflected during a fastening operation in order to subsequently latch by an inner clamping force behind a corresponding latching element, in particular in a latching recess, particularly preferably behind an edge of the pass-through recess. The latching element in particular forms an element with an undercut, which is configured to latch behind a corresponding element. The latching element is preferably configured to extend through the pass-through recess and to latch on the material handle body behind the pass-through recess. The latching element is realized, for example, as a snap-action tongue. Alternatively, it would also be conceivable that the latching element is arranged on the material handle body while a corresponding element and/or a recess is/are arranged on the connection unit. Both elements may in particular be attached to both units that are to be connected. The latching element is in particular latched to the corresponding element or latched into the recess. A positioning of the latching element during assembly is in particular brought about by a combination of placement and insertion. In particular, positioning and fixing is brought about in one action. During production, in particular the connection unit, preferably together with the application unit, and the handle unit are in each case produced separately and are joined subsequently during assembly by latching.

Various arrangements and/or design variants of the at least one latching element, deemed expedient by someone skilled in the art, are conceivable. If there are several latching elements, an arrangement is realized on a line or, in particular in the case of three latching elements, in a triangle. An arrangement in the triangle is in particular more stable and, in the assembled state, less wobbly than an arrangement in a line. Moreover, advantageous absorption of torsional and shearing forces is possible. However, in particular any arrangement of the latching elements is conceivable. If there are several latching elements, these may in particular be spring-loaded individually or they may be arranged on a common spring arm. The latching elements may in particular be arranged in a distributed manner on a common free-standing spring arm. However, it is also conceivable that several latching elements are provided each of which is arranged on a free-standing spring arm. The latching elements may latch in recesses in a main surface of the material handle body as well as in recesses in a side surface of the material handle body. The latching element may in particular extend over the entire thickness or width of the material handle body. It would also be conceivable that the latching element is arranged at the end of a longer tongue of the connection unit, in particular in order to generate lever forces for a stable arrangement. Alternatively, the latching elements may also be arranged on a rigid base of the connection unit. Alternatively or additionally an, in particular large, latching element may be provided.

In a preferred implementation the latching element is in particular realized in a mushroom shape with a stem section and a head section. Preferably, the mushroom-shaped latching element may be divided, thus comprising two or several springy sub-elements. The latching element or the sub-elements in particular comprise undercuts holding the connection unit to the handle unit. The material handle body of the handle unit is in particular held between a surface of the connection unit and an undercut on the latching element. This in particular allows providing an advantageous mechanical connection. Preferably, in particular one to six latching elements, preferably one to three latching elements and particularly preferably exactly one latching element are/is provided on the connection unit.

It is moreover proposed that the form-fitting element is realized as a rivet, in particular a plastic rivet. Preferably the form-fitting element is configured to extend through the pass-through recess and to be deformed, e. g. compressed, in particular melted (e. g. by heat, ultrasound or the like) behind the pass-through recess. This allows creating a mushroom-shaped rivet structure. The at least one pass-through recess is in particular realized so as to correspond to the at least one form-fitting element. The pass-through recess in particular forms a counter-contour for the form-fitting element. The form-fitting element is in particular realized as a cylindrical projecting shaft, wherein the form-fitting element in particular projects from a form-fitting contour of the connection unit or is arranged therein. Beyond this, the at least one form-fitting element in particular also serves for a positioning of the connection unit relative to the handle unit and/or for a positioning of the at least one form-fitting element relative to the pass-through recess. The implementation of the form-fitting element as a rivet in particular allows providing an advantageous mechanical connection, which is releasable only by breakage and thus cannot be reconnected for the intended use.

During production, in particular the handle unit and the connection unit are preferably provided separately. Then the handle unit and the connection unit are brought together, wherein in particular the form-fitting element extends through the pass-through recess. Following this, the at least one projecting shaft of the form-fitting element is deformed. Herein various deformation possibilities, deemed expedient by someone skilled in the art, are conceivable, such as, for example, by pressure, with the form-fitting element being pressed and/or slightly crushed; or e. g. by pressure and heat or ultrasound, with the form-fitting element being deformed and/or slightly crushed by means of a heated punch. Herein the pressure is in particular selected in such a way that controlled crushing takes place. When the pressure is applied, in particular an expansion of the compressed form-fitting element is controlled. Preferably a punch for this purpose is in particular not flat but concavely curved. A round shape of the contact surface in particular controls the flowing of the material of the form-fitting element. Preferably the pressure is applied explicitly only in the region of the at least one form-fitting element. Optionally it is conceivable that the punch is larger than the form-fitting element, that the punch is only partially contacted by the at least one form-fitting element and/or that in an end position the punch contacts the entire surface and not only the form-fitting element. For creating the rivet connection, in particular a counter-holder is necessary. In particular, the part to be pressed must be supported such that the pressure can be built up. Preferably the counter-holder is configured to abut on a bearing surface, in particular of the connection unit, and to provide a counter pressure. A geometry of the bearing surface on the body may in particular be integrated in an overall product such that it is not noticeable. Thus it is for example conceivable that instead of a large bearing surface several smaller bearing surfaces are provided, which do not attract attention. The bearing surface may preferably be situated in different ways. A structure and/or a position of the bearing surface may influence its size. The bearing surfaces are situated in a region suitable with respect to the structure of the product and to the necessary pressure that must be resisted. In particular, in the design of the connection unit it must be taken into account that when pressure is applied, there must be a counter-holder, respectively an abutment, such that the form-fitting element cannot give way. The size of the bearing surfaces (cumulatively) is at least the same, preferably larger than the cross-section of the part that is to be deformed.

Besides a heating of the form-fitting element by means of the heated punch, it is also possible to heat the entire product or at least the respective portion of the product, preferably without contact, for example, with a selective introduction of heat by means of a heating radiator, for example an infrared radiator.

Alternatively or additionally to the at least one form-fitting element of the connection unit, it would also be conceivable that the connection unit is connected to the application unit and/or to the handle unit by an adhesive connection and/or by a sealing connection. Preferably, the connection unit can be connected to the handle unit, in particular to the material handle body, by an adhesive connection. The adhesive connection can preferably be combined with a form-fitting connection. Preferably, in particular recesses and/or ridges and/or recesses which form a form-fitting contour are provided between the connection unit and the application unit and/or the handle unit. In particular, a form-fitting contour is provided on the material handle body. In addition to the orientation of the units, the form-fitting connection should in particular also receive a portion of the forces during the intended use, such that not all holding forces must be supported via the adhesive. In particular, harmful shearing forces can be absorbed. The form-fitting contour therefore in particular brings about a better, more stable connection. Furthermore, the form-fitting contour prevents impurities from accumulating. Preferably, a form-fitting contour corresponding to the form-fitting contour of the handle unit is arranged on the connection unit. A positioning of the connection unit relative to the handle unit is in particular brought about via the form-fitting contours. Alternatively, however, it would also be conceivable that a positioning of the units is brought about via tools for producing the connection.

Different adhesives, deemed expedient by someone skilled in the art, are conceivable for the adhesive connection. The adhesive must in particular be suitable for the connection of paper material and plastic. The adhesive may in particular be realized as a hot-melt adhesive. With the adhesive, in particular the open time and the setting time are relevant as characteristic variables. In the context of adhesive methods and adhesive techniques, the permissible time period from the start of the application of the adhesive until the joining parts are joined and the setting time begins is referred to as the open time. In the case of hot-melt adhesives, the open time is the time in which a liquid hot-melt adhesive is or gets distributed on the surfaces that are to be connected. Since the viscosity of a hot-melt adhesive increases after the application, the open time of hot-melt adhesives is temporally limited. The setting time is a term of adhesive technology and is the time period that an adhesive needs in order to achieve a strength required for an intended application, and/or the time that the adhesive takes for changing from a liquid state into a solid state. If molding pressure is built up during the adhesive process, it can be eliminated after the end of the setting time. With some adhesives, a post-setting time is still necessary until further processing, until the final strength has been achieved. After the setting time, the adhesive has achieved its inner strength, i.e. the cohesion. In a processing operation, in particular a long open time is favorable; during this time the adhesive can be applied and the different parts can be brought together. In the case of high values, the processing operation may in particular take place more slowly. As a rule, the open time cannot be accelerated from the outside and is given by the adhesive. A too long open time or too much time reserve may therefore slow down the entire process. The open time is preferably longer than the setting time. The open time is in particular from 2 seconds to 40 seconds, preferably from 5 seconds to 30 seconds. After the setting time, in particular the shorter will be the better. The setting time herein defines the time after the open time until the adhesive has fixedly joined the parts. During the setting time, in particular pressure is applied, for example, such that the parts are joined in the manner in which they are to be arranged eventually, or a positioning/position of the parts with respect to one another is ensured without the application of pressure. After the setting time, the parts are fixedly joined. The setting time is in particular from 0.5 seconds to 10 seconds, preferably from 1 second to 5 seconds. The setting time is preferably shorter than the open time. During the production, in particular a timing must be kept to when the adhesive is applied.

In the adhesive connection, in particular no full-area application is provided; a distribution of the adhesive mass takes place, for example, via the pressing during the open time. Different ways of application, deemed expedient by someone skilled in the art, are conceivable, for example as an adhesive dot, as several adhesive dots or as a bead. In particular, the application in points or as a bead with subsequent pressing usually will not result in a complete full-area distribution; therefore in particular a combination with structures for receiving forces is expedient.

The adhesive for the adhesive connection should preferably be suitable for oral hygiene and thus suitable for food products. Furthermore, an industrial application should preferably be possible, allowing an application in an automatic system. Furthermore, the adhesive and the materials of the handle unit and/or of the connection unit should be compatible. In particular, a tolerance-free assembly is enabled by means of an adhesive connection. Furthermore, gaps are in particular reliably filled with adhesive, since the adhesive is distributed under pressure. In particular, a standing of liquid in gaps can also be avoided. For better adhesion, surface structures on the handle unit and/or on the connection unit would also be conceivable, allowing better holding of the adhesive. Alternatively or additionally, a combination with a form-fitting connection would also be conceivable, allowing certain forces to be received mechanically.

Alternatively or additionally to the form-fitting element and/or to the adhesive connection, a plug connection would also be conceivable. The application unit may in particular be connected to the material handle body by plugging into and/or plugging onto the material handle body. Herein the body care product may in particular comprise a connection unit that is realized as a plug connection unit and configured for a releasable plug connection of the application unit with the handle unit. Herein it would in particular also be conceivable that the application unit is designed as an exchangeable head, wherein the plug connection unit is arranged directly between the application unit and the handle unit. The plug connection unit is in particular at least partially realized integrally with the application unit and/or at least partially realized integrally with the handle unit. Preferably the plug connection unit comprises at least one first plug connection element which is fixedly connected with the handle unit, and comprises at least one second plug connection element which corresponds to the first plug connection element and is fixedly connected with the application unit. In particular, the application unit can be separated from the material handle body via the plug connection unit. The plug connection unit is in particular configured to provide a releasable or non-releasable latching connection. The first plug connection element in particular forms a latching projection while the second plug connection element forms a latching recess corresponding to the first plug connection element. However, a vice versa design and/or another design of the plug connection unit, deemed expedient by someone skilled in the art, would also be conceivable.

Alternatively or additionally to the form-fitting element, a welded or sealed connection would also be conceivable. For example, ultrasonic welding methods, friction welding methods or sealing are suitable for this purpose. For the welded connection, in particular auxiliary means, such as for example varnishes and/or coatings on the paper material of the material handle body, may be used, which make it possible that the connection is created by the welding. For welding methods, in particular accessibility of the welding point, for example for sonotrodes, respectively sealing punches, is necessary.

Alternatively or additionally to the form-fitting element, the connection could be realized by overmolding with plastic. Herein a handle body may be completely or partially overmolded. For example, the connection unit and/or the base body of the application unit and/or the entire application unit may be realized with overmolding.

In the case of toothbrushes, for example the accommodation of a bristle platelet, a brush head with bristle holes or a brush head with injection-molded bristles may be realized by overmolding.

For the processing operation, the handle body is introduced into an injection-molding tool, where it is then overmolded.

When the handle body is introduced into the injection-molding tool, the body is held in the tool and is sealed in boundary regions between the overmolded portion and the non-overmolded portion. A sealing may take place for example by pressing, with the tool pressing onto the handle body and in this way somewhat deforms the handle body. The pressure that thus builds up on the handle body and acts between the handle body and the injection-molding tool serves for sealing.

The pressure exerted by the injection-molding tool, respectively by the cavity wall, is created by a difference of the mass of the inserted handle body and the cavity wall. In the case of a round cylindrical or hollow-cylindrical handle body, the difference in diameter is between 0.05 mm and 0.5 mm, preferably between 0.1 mm and 0.3 mm. In general, the cavity wall dips into the surface of the handle body by 0.025 mm to 0.25 mm, preferably by 0.05 mm to 0.15 mm, per surface area.

The connection between the handle body and the element created by overmolding of the handle body may take place in different ways.

The handle body may, for example, be connected to the overmolding body by a form-fitting and/or force-fitting connection. Herein the form-fitting connection may be implemented in different ways. For example, the handle body may be deformed during the injection due to the injection pressures, such that an undercut is formed which stabilizes the connection. The deformation is planned and goes only so far that there will not be any defects on the handle body. The form-fitting connection may also be formed by the injected plastic conforming to the surface and also entering pores and cavities in the surface, and thus a form-fitting connection is created. It is furthermore possible to create recesses on the handle body, for example in the form of blind holes, through holes, grooves, creases, which are flowed around or flowed through by the plastic. A fix connection is formed. The handle body may also be prepared insofar as the surface is roughened in the contact region in which the overmolding is realized. This allows improving the connection on the surface.

Alternatively, the overmolding and connecting by a form-fitting and/or force-fitting connection according to the above description may take place with a minimized overmolded body. The possibility is only used to create a basis for the further processing, for example in order to apply a soft component that does not easily connect to the handle body made of paper material. Thus, a hard component may be injection-molded which is attached to the paper material and on which subsequently the soft component is applied. Herein the hard component and the soft component form a material bond.

It is also possible to subject the paper material to pretreatment in order to enable an overmolding. Thus, a plastic film may be applied onto the paper material in the course a standard procedure of the paper processing. This plastic surface in turn enables an overmolding with hard component and/or soft component. The connection between the overmolding component and the plastic surface is in this case a material bond. However, the surface of the paper material may also be treated such that it is provided with an adhesion promoter, and in this way allows material combinations which would not be possible otherwise.

Also alternatively, it is possible to integrate auxiliary substances, which support the connection during the overmolding, directly in the paper material. This just enables the connection. For example, an adhesion promoter may be integrated directly in the paper material.

The overmolding may be used in particular for paper materials and, as has been mentioned, also for wood and bamboo.

During the overmolding, the hard component and/or soft component is injected with an injection pressure of 100 bar to 1,000 bar, preferably of 100 bar to 300 bar.

The bodies which are overmolded may or may not undergo special surface treatment before they are processed.

The bodies which are overmolded may have any shapes. For example, they may be flat and may be completely or partially overmolded in a sub-region. The body may also be a cylinder or a hollow cylinder (a tube), and overmolded. In this case, it is possible that the overmolding takes place at one end of the cylinder/hollow cylinder and, in the case of the hollow cylinder, it is possible that the mass of the overmolding material fills part of the cavity. If hollow cylinders are overmolded, the hollow cylinders are preferably supported in their inner region (cavity) by means of a prop. The prop is inserted into the cavity, supports the body and at the end also delimits the overmolding if the material component penetrates into the cavity.

In particular in the case of flat-shaped handle bodies and/or bodies that are overmolded, it may be necessary to support the body in such a way that the injected material component does not displace the handle body and that the overmolding can be realized all over the body. For this purpose, for example, small supports holding the body in position may have been introduced in the respective cavity of the injection-molding tool. The supports are, for example, small cylinders, and are later flowed around by the overmolding component.

It would also be possible to overmold the body in several steps. This has already been mentioned before, for example, with regard to injection-molding a second component onto a first component that is connected to the paper material. In addition to this two-dimensional option, it is also possible to inject in a first step insulated nubs that, on the one hand, serve as spacers to the cavity wall for a further overmolding step and, on the other hand, may also serve as fixing points for soft components. The soft component would be injected onto the first component and the paper in a second step.

The overmolded body may also be subjected to aftertreatment. For example, the body may be flocked.

A handle body made of paper material may, for example, be overmolded with a plastic (hard component or soft component) in order to form a flosser and to fix the dental-floss thread with the overmolding. The dental-floss thread is held in the end region of the handle body, i.e. in the head region, in a U-shaped geometry by two legs or arms. Herein the respective legs are in their end regions provided with plastic in which the dental-floss thread is fixed. Herein the synthetic material overmolds, on the one hand, a portion of the handle body and, on the other hand, the dental-floss thread, in each case on both legs, thus creating a connection of the parts.

During production, for this purpose a handle body made of paper material is provided, which is equipped with two sub-legs in the later head region. The ends of the sub-legs are prepared for an overmolding, preferably they are prepared symmetrically and equipped identically. Thus the respective leg may have a cross-section narrowing (in the later thread direction) in its end region, thus forming something like a pin. The pin in turn may be provided with one or several undercuts on its flanks, or the pin may be provided with a through hole, for improved securing of the connection between the handle body made of paper material and the plastic that is injected. The creation of undercuts and through holes is preferably brought about by punching, together with the punching-out of the remaining contour. Herein the plastic is applied only in the end region and does not extend over the entire body.

The overmolding should be applied such that the transition is continuous. After the overmolding a continuous body surface should form. The surface preferably continues in a stepless or continuous manner from the handle body made of paper material to the overmolded body. The material thickness of the handle body preferably remains the same between a portion made of paper material and the overmolded portion. The material thickness preferably increases by less than 50%, preferably by less than 30%, most preferably by less than 15%.

The plastic material thickness perpendicular to the longitudinal axis is less than 2 mm, preferably less than 1.4 mm, most preferably less than 1 mm.

In order to allow a more continuous design of the transition on the surface, respectively to attain a more constant material thickness, the handle body made of paper material may be pressed to the plastic in the interface region. As a result, a density of the paper material is increased and the material thickness is reduced. The reduced portion may then be filled up with plastic.

Herein the overmolding of the dental-floss thread is part of the prior art and, due to the processing with plastic, may be taken from the current production of flossers with handle bodies made of plastic. The ends of the dental-floss thread may be melted.

The plastic portion should be kept as small as possible. The portion of plastic and dental-floss thread should ideally not exceed 5% of the weight, such that recycling of the product in paper recycling, respectively cardboard recycling, is possible. Moreover, ideally a plastic based on sustainable material is used.

In the present case, the application may also be realized with a handle body made of wood or bamboo.

The advantage of this implementation of a flosser is that the paper material must be provided in only one layer and there is not a plurality of layers to be connected. This significantly simplifies production.

The described overmolding of the paper material may also be used for other products, such as dentist aids like tongue hold-down devices, or for cutlery such as coffee stirring sticks, ice spoons etc.

In principle, a combination of connection possibilities, in particular of the above-described connection possibilities, is also conceivable. In general, combinations of the various connection possibilities are possible. Thus, for example, a combination of a clamping connection and a snap connection is conceivable, wherein the material handle body is inserted, for example, into a recess of the connection unit and is latched at least by means of a form-fitting element. In addition, guidings may be provided in the connection unit, which guide the material handle body laterally. The guiding may also be realized as an additional form-fitting element, or the form-fitting element is only realized laterally. Furthermore, a combination of a rivet connection and an adhesive connection would also be conceivable. In the case of a body care product with a handle unit composed of several layers, it is for example conceivable that the connection unit is introduced and/or clamped between the layers of the handle unit, wherein the layers and the connection unit are connected. In such a case, the layers may for example be connected to the connection unit by means of a rivet connection, wherein form-fitting elements for a connection with the layers are in particular provided on mutually opposite-situated sides of the connection unit. Alternatively, the layers and the connection unit may also be connected merely by gluing, welding or sealing. However, a combination of individual methods of the mentioned methods is preferred, since the handle unit contains parts that do not adjoin the connection unit. In this context, a body care product with a double shell would also be conceivable, wherein two shaped shells of the handle unit are arranged opposite each other, such that a cavity is at least partly formed by the shells between them, and wherein the connection unit is clamped between the shells.

Beyond this it is proposed that the application unit comprises at least one base body, which is fixedly connected with the connection unit. In the case of a toothbrush, the base body of the application unit may be realized, for example, as a bristle platelet or as a brush head. Alternatively, it is also possible to select a connection with one or several degrees of freedom in the movement of the application unit (e. g. a rotationally fixed connection in the case of a wet shaver). The connection unit may be connected to the application unit, for example, by an adhesive connection. Further connection possibilities mentioned in this document are also possible (e. g. the described connection possibilities between handle unit and connection unit).

Preferably, the connection unit may also comprise the application unit, or an interface may be provided between the application unit and the connection unit. It is possible that the interface to the handle unit and the interface to the application unit are produced separately and are then connected. Thus, for example, the application unit and the connection unit may be produced and then connected, and after this the application unit that is connected with the connection unit may be connected to the handle unit. In particular, advantageously simple assembly is achievable in this way. In particular, a number of components may be kept low.

It is also proposed that the application unit comprises at least one base body which is exchangeably connected with the connection unit. The application unit may be connected with the connection unit, for example, via a releasable plug connection and/or latching connection. Preferably the connection unit comprises a first coupling element and the application unit comprises a coupling element that corresponds to the first coupling element. The first coupling element and the second coupling element in particular form a latching unit. A “latching unit” is here in particular to mean a unit with at least one latching element, which is elastically deflected during a fastening process in order to subsequently latch, due to an inner tensile force, behind a corresponding latching element. In this context, “releasable” is in particular to mean “non-destructively separable”. Preferably the application unit is in this context in particular realized as an exchangeable toothbrush head, as an exchangeable razor head or the like.

Furthermore, it is proposed that the application unit comprises at least one base body, wherein the form-fitting element is realized integrally with the base body of the application unit. Preferably the connection unit is realized integrally with the application unit. Preferentially the connection unit and the application unit are, for example, produced together in an injection-molding procedure. In particular, advantageously simple assembly is achievable in this way. In particular, a number of components may be kept low. It is moreover in particular possible to provide an advantageously stable body care product.

In an implementation of the body care product as a toothbrush, the application unit in particular comprises a bristle field. The bristle field may in particular be superimposed. Alternatively, it would also be conceivable that the application unit is riveted, the bristle field being designed in such a way that the arrangement of a counter-holder in the region of form-fitting elements is possible. The application unit in particular comprises a bristle platelet. The bristle field may herein be produced, for example, in an anchor punching, IMT, PTt or AFT procedure. It is herein in particular also conceivable that the bristle platelet is made of a paper material, wherein a connection to the connection unit can be created, for example, by gluing. Alternatively, it would be conceivable that the bristle platelet is made of plastic, in particular at least of a hard component, wherein a connection to the connection unit may be realized, for example, by welding, sealing, gluing, riveting, latching, deformation, snap-action or by a combination thereof. The bristle platelet may here in particular directly have a connection structure such as, for example, rivets, for a connection to the connection unit and/or directly to the handle unit. Alternatively, however, an implementation of the application unit without a bristle platelet would also be conceivable, wherein the bristle field may also be produced in an AFT procedure. In this case, in particular the molten pool (which is created on the fastening side, i.e. the side of the bristles that faces away from a usage) can be used for an anchoring of the bristle field. The application unit may herein be connected to the connection unit and/or directly to the handle unit by gluing, by clamping-in or by sealing, respectively welding, by riveting, by latching, by snap-action or by a combination thereof. Alternatively, the bristles may be punched into the bristle platelet by means of anchors or may be anchored in the bristle platelet without anchors by means of IMT or PTt. Herein conventional methods are known in which the bristle platelet is provided with bristle holes and bristles (e. g. anchor-punching procedures, IMT, PTt, etc.). The application unit may herein be connected to the connection unit and/or directly to the handle unit by gluing, by riveting or by sealing, respectively welding, or by a combination thereof. The bristle platelet may herein in particular directly have a connection structure such as, for example, rivets, for a connection to the connection unit and/or directly to the handle unit. Alternatively, the bristles may be injection-molded directly to a base body of the application unit (i. e. injection-molded bristles made of material for injection-molded bristles). The bristles and the application unit may herein be realized integrally. One or several material components may be used here. For example, a hard component and/or a soft component and/or a material for injection-molded bristles may be used. The application unit may herein be connected to the connection unit and/or directly to the handle unit, for example, by sealing, respectively welding, by riveting, by snap-action, by latching and/or by overmolding, or by a combination thereof. The base body of the application unit may herein in particular directly have a connection structure such as, for example, rivets, for a connection to the connection unit and/or directly to the handle unit, said connection structure being injection-molded directly to an underside of the base body of the application unit.

The application unit advantageously comprises at least one cleaning unit, in particular a toothbrush head, with bristles. The cleaning unit also advantageously comprises at least one bristle carrier, for example a brush-head base body, in particular the base body. It is further proposed that a base body of the application unit is completely made of a hard component and forms a bristle carrier. Alternatively or additionally, it is proposed that the body care product comprises a bristle platelet, which is accommodated in the base body and comprises a plurality of bristle bundles. For this purpose (exemplarily for the AFT method), firstly bristle platelets with through holes are produced by injection molding, and then bristles are guided through said through holes. Preferably the bristles are then connected, in particular melted, on a rear side at least in bundles, preferably to one another and/or to the corresponding bristle platelet. Bristle platelets provided with bristles in this way may then be connected, e. g. welded, sealed and/or glued, to a base body, in particular the connection unit, or may be mechanically connected to a base body, in particular the connection unit, by riveting, caulking, preferably by ultrasonic welding. For this purpose, the base body preferably has a corresponding structure for receiving/connecting, which may be used for a connection of the bristle platelet. Alternatively, the bristle platelet may be connected to the handle body directly, i.e. without an additional base body, by connection methods mentioned in this document. Alternatively, the bristle platelet may also be studded with bristles by means of other methods (anchor punching, PTt, IMT, etc.).

Beyond this, it would be conceivable that the bristle platelet is produced from a paper material. The bristle platelet may be punched from a paper material. For this purpose, one or several layers of paper material may be provided, which form the body of the bristle platelet. Then the recesses are formed; for this purpose a punching process, cutting, e. g. laser cutting, milling/drilling or another suitable method for a processing of paper material may be used. The bristles are in turn connected to the bristle platelet in the AFT procedure, wherein the latter may then subsequently be fixed in the corresponding recess in the handle unit or on the handle unit.

The application unit advantageously comprises at least one cleaning unit, in particular a toothbrush head, with bristles. The cleaning unit moreover advantageously comprises at least one bristle carrier, for example a brush head base body. Advantageously, at least some or all of the bristles are conventionally extruded bristles. Herein bristles may in particular comprise at least one hard component and/or at least one soft component. Preferably the bristles are manufactured at least partially or completely from polyamide (PA) and/or from polyester (PBT, PET, PTT), wherein any other materials (for example the hard components listed) are conceivable and sustainable materials listed in this document, in particular degradable materials (such as for example PLA or PTT), are also possible. Furthermore, it is conceivable that at least some of the bristles have a tapering and/or a variable cross section at the usage-side end. Preferably the bristles are made of a single, in particular also mixed, material. However, bristles with several components are also conceivable, which may in particular be producible and/or produced by means of at least one co-extrusion. The bristles may, for example, be producible and/or produced by extrusion, cutting-to-length and/or post-processing. In contrast to injection-molded bristles or rubber-elastic massage and cleaning elements produced by means of injection molding, conventional bristles are extruded, cut, machined (e. g. their ends being abraded and/or rounded) and inserted in the toothbrush handle by means of an adapted procedure, for example by means of the anchor-punching procedure or of an anchor-free procedure.

In particular, cylindrical or tapered bristles with a round cross section are possible, wherein any other cross sections, for example polygonal, triangular, rectangular, square, elliptical, star-shaped, trapezoid-shaped, cross-shaped, parallelogram-shaped, rhombus-shaped or any other cross sections are conceivable. In particular, different bristles may be used in a bristle bundle but it is also possible to use different bristle bundles, in particular each with a specific type of bristles. Herein bristles and/or bristle bundles may be arranged regularly but also irregularly. In particular, bristles and/or bristle bundles arranged in groups and/or such that they adjoin one another may, in particular alternatingly, differ with regard to at least one feature, for example a length, a diameter, a material, a color, a material hardness, a geometry, a tapering and the like. Preferably the bristles have a diameter, in particular perpendicular to their longitudinal axis, of at least 0.075 mm and/or of at most 0.25 mm. Advantageously, the bristles have a cross-section area, in particular perpendicular to their longitudinal axis, of at least 0.002 mm² and/or of at most 0.2 mm². In the case of bristles used in the cosmetics sector, for example bristles of an additional application element, it is also possible to use thinner bristles and/or bristles having a smaller cross section, in particular bristles with a diameter, in particular perpendicular to their longitudinal axis, of at least 0.025 mm and/or of at most 0.2 mm and/or having a cross-section area, in particular perpendicular to their longitudinal axis, of at least 0.001 mm² and/or of at most 0.15 mm². In the case of tapered bristles, in particular polyester (for example PBT, PET, PTT) is suitable as a material, wherein sustainable materials are also possible, wherein a tapering may be produced mechanically and/or chemically. However, other materials are also conceivable. Preferably the bristles are straight in the longitudinal direction; but bristles which are undulating and/or twisted and/or helix-shaped and/or twined in the longitudinal direction are also conceivable, and in particular combinations of different bristles are also conceivable. Furthermore, bristles with a smooth surface are conceivable, as are bristles with a structured and/or textured surface.

Furthermore, the bristles, in particular as bristle bundles, are preferably processed, in particular fastened to the bristle carrier, at least by an anchor-punching procedure or an anchor-free procedure, for example PTt or the like. Preferably the bristle carrier has a plurality of bristle receptacles, in particular holes for bristle bundles, which are in particular drilled and/or formed in an injection-molding procedure. In the case of anchor-punching it is for example conceivable that firstly a base body, in particular made of a hard component, preferably the brush head, is produced by injection molding, wherein advantageously blind holes for bristle bundles are formed during the injection molding. Of course, however, subsequent drilling of blind holes is also conceivable. Preferably, after this bristles and/or bristle bundles are folded and are fastened in respectively one blind hole by means of at least one anchor, in particular by punching. Loop punching is also conceivable. As has been described, the bristle carrier may be anchored directly on the handle unit or connection unit by overmolding. Herein the handle unit may consist, for example, of a paper material, a wood material or a bamboo material.

Alternatively, as has been mentioned, anchor-free methods (such as PTt, for example) are also conceivable, wherein advantageously bristles, respectively bristle bundles, are not folded. In such a case, bristles, respectively bristle bundles, have approximately half the length as compared to anchor-punching. For example, it is herein conceivable that the bristle bundles may first be melted individually and/or their bristle ends, in particular subsequently, for example for fastening, may be overmolded or may be fixed by material compaction of the brush head. Herein bristle bundles may advantageously be brought together.

It is further conceivable that the application unit is composed of a base body and a bristle platelet which is studded with bristles and/or with alternative cleaning elements. For this purpose, firstly bristle platelets with through holes are produced by injection molding, and then bristles are guided through said through holes. Preferably the bristles are then connected, in particular melted, on a rear side at least in bundles, preferably to one another and/or to the corresponding bristle platelet. Bristle platelets provided with bristles in this way may then be connected, e. g. welded, sealed and/or glued, with a base body, in particular a brush head, and/or may be mechanically connected by riveting, caulking, preferably by ultrasonic welding, or by a combination thereof. For this purpose, the base body, in particular the brush head, preferably has a recess into which the bristle platelet can be inserted. Alternatively, the bristle platelet may also be superimposed on the surface of the base body. In this context, the anchor-free tufting method (AFT), which in particular enables a bringing together of bristle bundles, must be mentioned as a known production method. In particular, a side which is placed into a recess of the base body or onto the base body, pointing towards the rear side of the body care product, is referred to as the underside of the bristle platelet. Correspondingly, the upper side of the bristle platelet points towards the front side of the body care product. The base body may consist of a hard component made of a sustainable material or of a paper material. Alternatively, the base body may also be omitted and the bristle platelet may be connected directly to the handle body by the methods mentioned.

Alternatively, the bristle platelet may be produced from a paper material or from a material that consists of fibers, respectively from pulp or from wood. Outside the bristle field, the bristle platelet made of paper material has a peripheral region which is provided with a connecting means such as sealing wax, glue or the like, and which enables a sealing and/or anchoring of the bristle platelet with a handle unit or with a base body that is, for example, made of a paper material or of a hard component or of a sustainable material. Herein in particular the mentioned peripheral region is connected to the handle unit and/or to the base body. The peripheral region of the bristle platelet may also be provided with an effective step, such that a recess is formed on the rear side of the bristle platelet, which ensures that the melt of the bristles does not extend beyond the edge of the bristle platelet and/or does not cover the peripheral region either.

A bristle platelet made of paper material or wood is formed by a suitable method from planar paper or from wood sheets (e. g. veneer). Possible methods are, for example, punching, laser cutting or cutting. The planar region of the bristle platelet has a thickness of 0.1 mm to 3.0 mm, preferably 0.3 mm to 2.0 mm, particularly preferably 0.5 mm-1.5 mm.

Alternatively, a preferably one-piece bristle platelet can be realized that is produced together with the bristles/cleaning elements. Herein everything (e. g. a brush head and/or an accommodation for a bristle platelet and/or a bristle platelet and/or bristles) may be produced in one injection-molding operation. Material for injection-molded bristles is preferably used for this purpose. In addition, a hard component and/or a soft component may also be used. This bristle platelet, too, may in turn be connected to the base body by a large variety of connection techniques mentioned, for example by gluing, sealing, welding, caulking, riveting, or a combination thereof.

Alternatively, the production of an element provided with bristles, without a bristle platelet, may be provided. The production and/or bristling of this element is brought about in a manner similar to the production of a bristled bristle platelet. Bristles are provided in the required configuration and are melted at their fastening-side ends. In such a case, the melting must be such that a melt carpet of the bristle component is formed over all the bristle bundles and any further elements. The bristled element is then mounted and/or connected to a brush body/base body. Herein, in an implementation variant, the element is not equipped with an additional bristle platelet, but is inherently stable due to its own melt carpet. The melt carpet is created from the fastening-side melted ends of the bristles. The melt carpet essentially forms a surface, respectively a plate, and thus serves as a basis for the connection to the base body or directly to the handle unit. The melt carpet may also be shaped such that it comprises connection elements in the form of projections, rivets, ridges, or other form-fitting elements. The melt carpet with the bristle bundles may be produced by means of AFT or by another anchor-free bristling method. This element, too, may in turn be connected to the base body, respectively the handle unit, by a large variety of connection techniques mentioned, for example by gluing, sealing, welding, caulking, riveting, or combinations thereof. Herein the base body may be provided with a recess, such that the melt carpet can be sunk therein and fastened. In principle, however, a fastening directly on the surface of the handle unit is also conceivable.

Instead of a bristle platelet, the bristles/cleaning elements may also be injection-molded directly to the base body and/or to the handle unit. Thus a base body, respectively a handle unit, made of a paper material would also be directly overmolded and the bristle platelet and/or the injection-molded bristles integrally attached thereto would be fixed on the base body and/or the handle unit.

A production, in particular an injection molding, of a brush head with through holes for bristles is possible as a further method for anchor-free bristling. Bristles may then be guided through the through holes and may be melted on a rear side, in particular with one another and/or with the brush head. Preferably after this an overmolding of the melted regions and/or of the brush head is carried out, in particular an overmolding with at least one soft component.

Alternatively to the separate bristle platelet, the bristle platelet is realized as part of the base body made of a paper material or wood, or is integrally connected therewith. Analogously the brush head is also in this variant as a possible implementation variant provided with through holes. The bristles may also in this case be guided through the through holes and may be melted on the rear side at least in bundles, in particular melted with one another and/or with the brush head. The melted regions and/or the rear side of the brush head may then be closed or covered with a cover element made of paper material by connecting the cover element to the brush head. In this regard once again the above-mentioned connection techniques are suitable.

It is moreover conceivable to firstly produce a brush head with blind holes, for example by injection molding and/or by drilling the blind holes and/or by cutting/punching. Bristles are in this case in particular put together into bundles and are melted and/or connected in another way at one end. The brush head is then heated and/or moistened with water or steam. After this bristle bundles may advantageously be introduced into the blind holes on the connected side and may be anchored by pressing and/or compressing the brush head. Herein in particular the heated and/or moistened blind holes, respectively the brush head, deform and/or compress such that the bristle bundles are anchored therein. At the same time the brush head may also be deformed. This method may be used for hard components, preferably by additional moistening, but also for paper material and/or wood.

Preferably in an injection-molding process, in particular a two-component and/or multi-component injection molding, materials of injection-molded bristles do not form a material bond with other soft components and/or hard components and/or paper material and/or wood and/or bamboo and/or other materials of the body care product. Rather, injection-molded bristles are preferably connected by a form fit connection, for example via at least one undercut and/or via at least one breakthrough on the base body of the application unit and/or via at least one at least partial overmolding with soft components and/or hard components, wherein in particular a wasting connection and/or a shrinking connection are conceivable. Furthermore, additionally or alternatively during the overmolding, due to the heat and the injection pressure, the plastic may at least partially enter the surface of the paper material, wood material or bamboo material and/or may at least roughen the surface, thus creating a fix connection.

However, a connection via at least one material bond, in particular with affine and/or compatible plastic materials and/or components, is also conceivable.

In principle, a one-component, two-component and/or multi-component injection molding is conceivable for all the possible injection-molding processes mentioned. As has been mentioned, materials used, in particular different soft components and/or hard components and/or material for injection-molded bristles, may get connected and/or be connected by material bond and/or in a form-fitting manner. A realization of articulate and/or mobile or flexible connections via suitable injection-molding steps is also conceivable. In principle, for example, hot-channel methods, cold-channel methods and/or co-injection methods are suitable.

Alternatively or additionally to a brush head studded with bristles, the application unit may also comprise at least one tongue cleaner and/or at least one alternative cleaning and/or massaging element. These may in each case be made of a soft component, of a hard component or of a combination of soft component and hard component, and/or may advantageously be producible and/or produced by injection molding.

Material for injection-molded bristles: Preferably injection-molded bristles are made at least partially and advantageously completely of a thermoplastic polyurethane elastomer (TPE-U). Herein it is conceivable to use a modified polyurethane elastomer (TPE-U), which may in particular be modified with regard to improved flow properties and/or fast solidification, in particular fast crystallization, advantageously already at rather high temperatures. Of course, however, other materials for injection-molded bristles are also conceivable, for example thermoplastic polyester elastomers (TPE-E), thermoplastic polyamide elastomers (TPE-A), polyethylene (PE), for example in the form of low-density polyethylene (LDPE) or linear low-density polyethylene (LLDPE), or the like. Depending on the desired bristle properties, in principle the listed hard components and/or soft components may also serve as material for injection-molded bristles.

Materials for injection-molded bristles advantageously have a Shore D hardness of at least 0, particularly advantageously of at least 30, and/or of at most 100 and advantageously of at most 80. In particular, a Shore D hardness of a material of injection-molded bristles is advantageously higher than a Shore D hardness of other soft components used, for example for handle elements, massaging elements, further cleaning elements or the like. The materials used for the production of injection-molded bristles may be sustainable materials.

In principle, it is also conceivable to use water-soluble polymers, for example for hard components, soft components, injection-molded bristles, tongue cleaners or other elements of the body care product.

Likewise, for the hard component, the soft component and/or the material for injection-molded bristles bioplastics may be used, which may be obtained, in particular at least partially, from sustainable raw materials and/or may be biodegradable, in particular compostable, and/or may consist of a recycled and/or recyclable material. Preferably the material is in particular produced from a plastic. Preferably the biodegradable, in particular compostable, and/or recycled and/or recyclable material is realized as a bioplastic, in particular as a plastic based on sustainable raw materials and/or as a biodegradable plastic. The material may therefore in particular be fossil-based and biodegradable, such as for example PVOH, PCL, PBAT, PET or PBS, based on sustainable raw materials and biodegradable, such as for example PLA, PHA, cellophane or starch blends, or based on sustainable raw materials and non-biodegradable, such as for example Ca, Bio-PE, Bio-PP, Bio-PA, Bio-PET. Various bioplastics, deemed expedient by someone skilled in the art, are conceivable, such as for example starch-based bioplastics, cellulose-based bioplastics, polyhydroxyalkanoates, such as in particular polyhydroxybutyric acid (PHB), polylactic acid (PLA), aliphatic and/or aromatic co-polyesters, or further bioplastics, such as for example lignin-based bioplastics. Preferentially the application unit and/or the handle unit may be made largely of a biodegradable, in particular compostable, and/or recycled material. Preferably the body care product consists of only one component. In particular, the body care product comprises a hard component. The material handle body of the handle unit may consist at least partially of the hard component. The hard component and/or the soft component and/or the material for injection-molded bristles advantageously consists of a bioplastic, which may in particular be obtained from sustainable raw materials. Suitable raw materials herein are in particular corn, hemp, sugar, castor oil, palm oil, potatoes, wheat, sugar cane, caoutchouc, wood, the castor plant/ricinus and the like. Appropriate possible base materials could be for example cellulose, starch, lactic acid (PLA), glucose, chitin, chitosan or the like, from which in particular respective bioplastics can be synthesized.

In an implementation of the body care product as a razor, the application unit in particular comprises a razor blade, wherein the razor blade may be produced so as to be directly connected with the connection unit, and may be realized in exchangeable fashion, wherein the exchangeable mounting of the razor blades takes place at the connection unit. Alternatively, the razor blade may also be attached directly to the handle unit. In this case, the connection points would have to be treated specially, for example by compaction and/or surface treatment of the paper material.

Preferably the application unit is realized at least partly integrally with the connection unit. The connection may be implemented so as to be rotationally movable. Preferably, in an implementation of the body care product as a razor, a plurality of anchoring points are provided between the connection unit and the handle unit. Here, in particular three connection possibilities are conceivable in combination, namely riveting, clamping-in and/or lateral holding; additionally or alternatively gluing, welding or sealing are also conceivable. Herein a universal interface design in particular allows easy change of model, such that a connection unit plus application unit can be used for different handles. The different handles may in turn be combined with the same connection unit.

In an implementation of the body care product as a brush, the bristles of the application unit may in particular be injection-molded directly to the connection unit and/or to the handle unit.

It is further proposed that the material handle body of the handle unit has at least one head region, which is configured for a connection to the application unit, wherein the material handle body of the handle unit has in the head region at least elements, for example in the form of a depression, which are configured for a positioning and/or orientation of the connection unit relative to the handle unit. The elements and/or the depression in particular form/s a form-fitting contour. The depression is in particular realized as a groove in the material handle body, which extends parallel to a longitudinal axis of the handle unit. The material handle body comprises the form-fitting contour, which is realized such that it corresponds to a form-fitting contour of the connection unit. The form-fitting contour of the connection unit to the adjoining handle unit creates a certain form-fitting connection by the geometry next to the form-fitting element. Preferably, in particular recesses and/or ridges, which respectively realize the form-fitting contour, are provided between the connection unit and the handle unit. The form-fitting contour is in particular meant to receive a portion of the forces, such that not all forces must be supported by a rivet connection and/or an adhesive connection. In particular, harmful shearing forces can be absorbed. The form-fitting contour therefore in particular brings about an improved, more stable connection. Preferably, a form-fitting contour corresponding to the form-fitting contour of the handle unit is arranged on the connection unit. A positioning of the connection unit relative to the handle unit is in particular brought about via the form-fitting contours.

Beyond this it is proposed that the material handle body of the handle unit consists of at least one, preferably precisely one, layer of a paper material. Preferably the material handle body is produced in a one-part implementation. The paper material can in this case be realized in a single-lay or multi-lay implementation. Multi-lay paper material may consist of several lays of paper of different thickness and/or different properties and/or at least partly of different materials; however, it is also conceivable that the listed aspects (thickness, properties, material) of the individual lays are at least partly identical. Preferably several lays of paper may form a layer, wherein the body may consist of one or several layers.

If several layers are used for the body care product, for example with the connection unit and/or the application unit being fixed between the layers, the layers must be applied onto one another in such a way that rounded edges shall be provided towards the outside and sharp edges of the layers shall be provided on the inside and/or against one another. This approach is to be considered mostly if the layers of the handle unit are punched before they are connected, respectively before the connection unit and/or the application unit are/is introduced.

Herein outer lays may have different water-repellent properties than inner lays. Outer lays may have a different coloring than inner lays. Individual lays or all lays may consist essentially of fresh fiber, respectively primary fiber. Individual layers or all layers may consist essentially of secondary fiber. The paper material may have between 3 and 15 lays, preferably between 5 and 12 lays. The lays may herein be produced by gluing, laminating or pressing together, in particular also without the use of adhesive.

Herein in particular not all lays must consist of the same material. It is for example conceivable that printed outer lays could be made of thinner paper and/or cover lays could have different properties than middle lays. Thus, for example, an outer lay may already have a coating, varnishing or printing before a processing operation. Alternatively or additionally, different colors of the layers in the sandwich are also conceivable, such that the layers are clearly visible.

The finished body care product may be formed at least in individual regions from several layers of the paper material. For example, two, three, four, five or six layers may be formed. The number of lays is multiplied accordingly.

A fiber orientation of the paper material is preferably in the longitudinal direction of the handle unit, such that bending is reduced. The grammage of the paper material, in particular of the entire layer, is in particular between 700 g/m² and 2,500 g/m², preferably between 900 g/m² and 1,700 g/m². In accordance with DIN 6730, different paper materials can be distinguished with regard to grammage. Thus up to 225 g/mm² it is paper, whereas with 225 g/mm² and more it is referred to as paperboard. Colloquially up to 250 g/m² is referred to as paper, from 150 g/m² to 600 g/m² as cardboard and with 500 g/m² and more as paperboard. For cardboard, for example, solid fiberboard or corrugated paperboard is conceivable. There are in particular different variants of cardboard, in particular also on a recycling basis. Preferably the paper material, in particular in the case of cardboard, particularly preferably in the case of solid fiberboard, consists of fresh fiber and/or secondary fiber. The fresh fiber, respectively primary fiber, is realized as a new material such as for example FSC wood. The secondary fiber is in particular realized as a recycled paper and/or recycled cardboard. Alternatively or additionally, further fillers such as grass for grass paper, bamboo, sugar cane are also conceivable. Paper is produced from fiber materials, which are nowadays obtained mostly from the raw material wood. There is in particular also the possibility of wood-free cellulose. However, the preferred basis of the paper material is cellulose, respectively wood-based pulp. Possible types of basic wood are for example birch, poplar, beech, spruce, pine or eucalyptus. Alternatively, the paper material may also be obtained from grass, bamboo or papyrus. The most important fiber materials are pulp, wood materials and waste paper material. Some kinds of waste paper may be rather less suitable for use in oral hygiene because of printing ink. In addition to the fiber material or to a fiber material mixture, the material may often also comprise fillers and further additives. The paper material as a whole or at least individual lays may have a recycling fraction of at least 80%, wherein the paper material as a whole or at least individual lays may comprise a portion of primary fibers. By using new material, respectively primary fiber, the properties of the paper material may be kept in a narrower range and, among other things, better tear strength and thereby stability is achievable. Different paper materials, deemed expedient by someone skilled in the art, are conceivable. It is also conceivable that the paper material is painted, i.e. coated, or unpainted and/or structured, such as for example by a 3D contour. By means of structuring and/or coating, in particular a grippability of the body care product can be improved. In particular, the structuring may be realized both in the paper material itself, for example by a surface treatment and/or an embossing, as well as in a printing.

As has already been mentioned, the paper material may be substituted by other sustainable materials such as wood or bamboo. Possible kinds of wood are listed above, among other things.

Preferably, production of the paper material is realized free from an injection-molding or extrusion process, except for fiber casting or fiber injection molding which is carried out at lower temperatures and/or lower pressure as compared to the processing of thermoplastics. The temperature is in the range of maximally 200° C. and the pressure is between 10 bar and 40 bar, preferably between 20 bar and 35 bar. In particular, no melting process of thermoplastics is involved in the production of the paper material.

The paper material may in particular be provided in various ways, deemed expedient by someone skilled in the art, such as in particular in the form of sheets, in the form of page layouts, from a roll, in block form or as a granulate or as a base material. The block may herein, for example, consist of a cardboard material and may be configured, for example, for mechanical processing, such as milling. This allows creating a volume body that is implemented in a less flat manner.

The paper material may have different densities and/or thicknesses depending on an application. Possible grammages are, for example, a cardboard for a toothbrush, a razor or a brush. Possible densities and/or thicknesses per layer may be 700 g/m² to 2,500 g/m², preferably 900 g/m² to 1,700 g/m². If several layers are used, the sum of the grammages varies in the corresponding range. One, two, three, four or five paper material layers may be used. The layers may have different grammages and different paper materials. The layers may comprise plastic films. Preferably the plastic films are covered on one or both sides by layers made of paper materials. The layers may be created by folding a planar paper material. Two or more foldings may be produced. The foldings may be sequential and/or rolling or may change sides in a respectively alternating manner (accordion-wise).

Preferably the paper material may be realized as a wood-free painted cardboard. Overall, at least one surface of the paper material may be respectively painted, printed, embossed or the like, depending on an application. For the paper material, in particular a high tear strength and at least on an application side a high water barrier are advantageous. The tear strength is between 20 N and 100 N, preferably from 30 N to 60 N.

For a testing of the water barrier, respectively water resistance of paper materials, these may be subjected to an immersion test. For this purpose, the paper material is cut into a predefined strip, for example with a length of about 100 mm and a width of about 30 mm. The weight of this strip is measured. Then the strip is immersed in a temperature-controlled water bath. The water bath has a temperature in the range of the human body temperature and is thus between 36° C. and 38° C. The strip is immersed 50 mm deep for a predefined time period. The time period is between 5 seconds and 180 seconds; 180 seconds is given as the recommended cleaning time with a toothbrush. After this, the strip is removed from the water bath and the water is knocked off. Directly after the knocking off, the strip is weighed. The weight difference shows how much water has been absorbed in the paper material. The weight differences between dry and wet paper materials depend on a wide variety of factors. The structure of the paper material, its coating and treatment, the water temperature, the shape of the cut surfaces etc. contribute to the values.

The best, respectively suitable paper materials have, with 60 s soaking and/or dwell time in water at a temperature of 36° to 38°, a water absorption of 5% to 12%, preferably of 6.5% to 9.5%, directly after the soaking.

The non-paper fraction of the total weight of the body care product is less than 10%, preferably less than 5%, most preferably less than 3%. The non-paper fraction is composed, for example, of plastic film and/or bristles and/or injection-molded parts and/or metal parts such as wire or metal anchors.

The paper material may also be subjected to additional treatment, in particular to achieve additional properties. For example, a coating may be provided on a surface of the paper material in the form of a varnish, of an adhesive, of a physical and/or chemical treatment for the water resistance or as a finishing.

Beyond this, fillers for the paper material are conceivable. In particular, insoluble fillers and soluble fillers are conceivable. Possible fillers are antibacterial additives, e. g. silver particles, polymers for improved water resistance such as, for example, in paper cup production, abrasive agents (increasing the friction coefficient), for example for improving the cleaning performance if the paper material is used in the cleaning unit, or for improving the holding, respectively the haptics, if the paper material is used in the handle unit, and/or magnetic particles for a magnetic paper, for example for a holding and/or storing and/or fixing of the body care product.

Further fillers which dissolve during use may be the following active substances, which are bound into a corresponding carrier and are released:
a) Active substances with toothpaste-like effect: sorbitol, aromas, hydrated silica, sodium lauryl sulfate, sodium monofluorophosphate, creatine, zinc sulfates, triclosan, glycerol, sodium saccharin, propylene glycol, disodium phosphate, aluminum oxide, trisodium phosphate, sodium fluoride, betaine, titanium dioxide, carboxymethylcellulose, tetrasodium pyrophosphate, etc.
b) Active substances with antibacterial effect: sodium bicarbonate, citric acid, phosphoric acid, sodium carbonate, potassium carbonate, sodium perborate, sodium hexametaphosphate, sodium benzoate, sodium stearate, etc.
c) Active substances for indicating the cleaning success by a coloring of the plaque on the tooth surface: glucose, maltodextrin, magnesium stearate, aroma, saccharin, microcrystalline cellulose, etc.
d) Active substances which give off taste
e) Active substances which give off smell

Other fillers may be colorants; in the case of oral hygiene products these are preferably food colorants.

Such paper materials are already known, for example, from cardboard for drinking cups of the company Storaenso, said cardboard being suitable for food products, waterproof, compostable and printable. Furthermore, from the company Kotkamills cardboard is known which is made waterproof by dispersion and is still fully recyclable and compostable. Furthermore, cardboard tubes are already known.

The paper material may have been pretreated, e. g. finished. A “finishing” is here to mean a surface treatment of the paper material which, for example, enables higher-quality appearance of the paper material. A finishing may be applied during or after the printing process of the paper material. A finishing may be applied during or after the process of assembly of the paper material with other elements of the body care product. The finishing may in each case be applied/realized partially in one or several points or also over the entire surface. The finishing allows, for example, realizing metallic effects, optical distinctions or also product inscriptions. A suitable coating may also allow a reduction of the water absorption of the paper material. The coating may here be applied for example after a processing of the handle unit, e. g. a punching and/or forming, by immersion in a bath with the respective coating medium. Further possibilities are, for example, the spraying-on of a coating and/or that, for example, an uppermost lay of the material handle body already has a coating and/or a varnish on its outside. The coating and/or the varnish must here in particular be suitable for food products. In this way the paper material and/or the body care product produced from the paper material may also be suitable for multiple use. A finishing may, for example, be a varnishing; here, for example, UV high-gloss varnish, structural varnish, soft-touch varnish or glitter varnish may be used. The finishing may also be an at least partially applied flocking. The finishing may be an embossing, for example a silver foil embossing or a hologram embossing. The finishing may be a fragrant varnish. As a further possibility of the pretreatment, for example, the embossing of the paper material is provided.

The mentioned flocking of the paper material may also assume functions of the product. Thus the flocking may be realized in such a manner that it can be used as an application unit. In this case, the flocking is quasi a bristling method for equipping the product. The flocked place has a different surface roughness and may thus be used as an applicator or as a cleaning element. In addition, it is also possible to provide certain gripping zones on the handle body with a flocking, such that the flocked place has a different grippability, for example an improved grippability, and in this way serves as a holding/gripping zone.

Preferably the material handle body is layered from a sheet, from a roll or from a page layout. The layer of the material handle body may herein in particular be realized by bringing several sheets and/or rolls together to form a large sheet or a large layer. Subsequently the layer may be molded. The layer may herein be deformed out of the plane, wherein in particular deformation angles of at least 10°, preferably at least 20° and particularly preferably at least 30° are possible with respect to a main extension plane of the layer. Too large angles will in particular make the paper material fan out at the cut and/or punched edges, and/or will make individual layers tear at the bending point. Test series have shown that with the proposed layer thicknesses and/or grammages, deformation angles below 60°, preferably below 45°, particularly preferably below 35°, are expedient.

For the same reasons, during the deformation out of the plane radii are preferably also provided at resulting edges. These are preferably selected such that they are not too small. Intensive tests have shown that minimal radii between 0.1 mm and 3.00 mm minimize problems listed.

Larger radii of up to 20 mm, preferably up to 10 mm, may of course also be used in the deformation. They are used in particular to form a round concave, respectively convex (partial) profile of the handle unit in the extension direction. With the deformation out of the plane, the stability of the paper material is increased in this region and a U, V, T, or L profile is formed. The larger the selected radii, respectively the smaller the selected angle, the more the stabilizing effect will disappear.

The paper material may also be compacted at least partially, respectively in portions, by pressing, which means that the deformation of the layer or the layers may at the same time bring about a compression of the volume. A higher compression will result in a higher density, a higher stability and also less risk of water entry. In combination with a deformation, a compaction, respectively a compression, may significantly improve stability locally. A compression takes place by at least 20%, preferably by at least 35%, particularly preferably by up to 50% relative to the initial thickness. The compaction is preferably carried out in places of the handle unit which realize a different function, respectively an additional function. For example, in this way zones of the paper material may be reinforced in which other elements are attached and/or fixed (e. g. connection unit, application unit, razor blade, brush head, etc.).

It is also possible that zones of the handle unit are reinforced which perform a function, e. g. a scratching edge, a scraping edge, a cutting edge, a toothpick tip, haptic elements, e. g. for finger rests, characters, logos, etc.

The edges of the handle unit made of paper material may likewise be compressed, respectively compacted, and may thus be rounded such that they are less sharp-edged.

The body care product that is realized as a toothbrush may have integrated a further function as part of the handle body on its side situated opposite the application unit. For example, a toothpick tip may be realized. In this way, the body care product may perform two product functions, respectively in the present case two oral hygiene functions. The toothpick tip may herein be realized as a further symmetrical part (symmetry with respect to the longitudinal axis of the handle unit) of the handle body, such that the toothpick tip is arranged on the longitudinal axis of the handle unit. Alternatively, it is also possible to shape the toothpick tip asymmetrically (with respect to the longitudinal axis of the handle unit), for example in contrast to the remaining portion of the handle body. Herein in principle the remaining portion of the handle body may also be realized asymmetrically (with respect to the longitudinal axis of the handle unit). The asymmetrical toothpick tip may, for example, be formed on the handle body in a sickle-shape.

It is also conceivable that the at least one layer is pretreated, printed or finished before a processing operation (in this regard, see the further explanations in this document). The pretreatment, printing and/or finishing may concern only one lay; it may also be realized with an entire layer on one side or on both sides. As a pretreatment, for example, embossing is conceivable, with the aim of producing a structure on the surface of the final product. Alternatively or additionally, a coating is conceivable as a pretreatment. For example, an inner side of each layer could be configured for an adhesive bonding while an outer side has a protection against moisture, respectively water. Each layer may therefore have two sides with different coatings. Alternatively or additionally, a printing is conceivable as a pretreatment. It is further conceivable that the at least two layers are subjected to aftertreatment after production and/or assembly. As an aftertreatment, for example, a coating, a printing, a coating of the application unit and/or a lamination are/is conceivable. Preferably, the application unit is connected directly to the material handle body. In an implementation variant, the application unit and/or connection unit is in particular inserted between the layers of the material handle body and is connected together with the layers of paper material, and/or is anchored between the layers during the connecting of the layers.

Preferably, the handle body may be provided with a decoration and/or a coating. The decoration and/or the coating may here extend over the complete handle body or may extend only over regions. Herein also different decorations or coatings on different parts of the handle body are conceivable. The decoration and/or the coating may be applied on the front side as well as on the rear side. The decoration and/or the coating may be applied over the entire surface or only partly. Thus also merely a partial coating for water resistance in respectively exposed regions would be conceivable. It is thus possible, for example, that only cut edges or—in the case of toothbrushes or razors—merely the region close to the connection unit and/or to the application unit may be partially coated. The decoration and/or the coating may influence the properties of the handle body that is made of paper material. In addition to the primary function of the coating and/or of the decoration, the entire handle body will also become more rigid. Thus, soft paper may be made more stable and more rigid by the treatment. For an increased stability of the handle unit, it is expediently—in combination—at least partially deformed, compacted and coated.

The decoration may also be applied, for example, by cold embossing/hot embossing, by printing such as for example inkjet, laser or digital printing, by lamination, by partial combustion/carbonization, e. g. by means of hot punch or laser, or by a combination of printing and coating. In a combination, the printing is, for example, not water-resistant, scratch-resistant or the like, which in turn may be remedied by an additional coating.

The decoration and/or the coating may be applied to a lay or a layer prior to assembly, in particular prior to the punching and/or prior to a deformation. This in particular has the advantage that a printing and/or coating may possibly already take place during paper production. Moreover, an application may take place in the flat state before a deformation. In order to avoid damage to the decoration or the coating, it would be conceivable that punching edges, bending edges, folding edges or potential tear regions are not provided with the application, and/or are recessed.

Alternatively, the decoration and/or the coating may be at least partly creased at the later bending edges, folding edges after application and prior to assembly, in particular prior to the deformation. As a result, the decoration and/or the coating should tear less when the effective deformation takes place.

Furthermore, the decoration and/or the coating may be at least partly slit at the later bending edges, folding edges after application and prior to assembly, in particular prior to the deformation. Thus the decoration and/or the coating is cut in a suitable place, and the coating tears less during the deformation. A disadvantage of this is that inner layers of the paper material are exposed, and this may have disadvantages both optically (different coloring visible) and function-wise (interruption of the coating). However, this could be at least partly bridged by a further coating following the deformation.

Damage to the decoration may likewise be reduced if the decoration is applied to the paper material on a flexible layer. This means that the flexible layer, made of a plastic component such as e. g. polyethylene, polyamide or a sustainable plastic (for example in the form of a film), is applied or laminated onto the paper material, and the latter is then deformed. Due to the flexibility of the layer, damaging is avoided or at least largely reduced.

Alternatively, it would also be conceivable that an application takes place at least partially after the deformation of the handle body, wherein an application must then be made onto a 3D geometry. Herein, however, an application could be realized over several components wherein, if parts mutually cover one another, it is possible that covered regions are not coated.

Preferably, a surface for the application of markings is provided on the handle body. The markings may, for example, be introduced as a printing or as a hot/cold embossing, or as a laser inscription. An embossing may, for example, be introduced directly during the shaping of the remaining structure. Therefore no special embossing surface is necessary. An arrangement of the markings is realized in particular in a region with an overflowing shape without contour steps, i. e. with a continuous geometry. Preferably the marking is for example arranged in a depression, in particular integrated directly in the structure of the handle body, or arranged offset on a pedestal.

In a preferred implementation, the material handle body is produced in particular by bringing several paper lays together to form a layer, then applying a finishing, e. g. printing/varnishing, following this punching, forming, compacting of the handle body and then applying a protective layer, e. g. a protective varnish, e. g. by immersion, spraying of the handle body with a coating agent for water resistance. The individual process steps are at least partly optional and may, as described above, also be carried out in different sequences. Depending on the body care product, two or more layers may then be brought together to form a handle unit.

A foil lamination, foil lining and/or hot-foil embossing may also be used. Paper materials may herein be produced inter alia from several lays with a film. Herein matt foils, gloss foils or also structural foils may be used. As a result, in particular the service life of the surfaces is extended and the surfaces gain an optically attractive appearance. Embossings/embossed foil printing may be used, wherein in particular planar or structural embossing is possible. A lamination or a sealing-in may also be used for the finishing.

Herein in particular various implementations of the body care product, deemed expedient by someone skilled in the art, are conceivable. The material handle body may in particular be produced from one or more sheets of a paper material and/or rolled from rolls and/or rolled as a roll of strips and/or of a tape. In the case of several layers, the layers of the material handle body may be connected to one another, for example, by gluing, by sealing, by welding and/or by lamination. It is also conceivable that the at least one layer is pretreated before a processing operation. As a pretreatment, for example, at least partial embossing of the paper material is conceivable, with the aim of producing a structure on the surface of the final product and/or a cavity in the handle body. Alternatively or additionally, a coating is conceivable as a pretreatment of the paper material. For example, an inner side of each layer of the paper material could be configured for adhesive bonding while an outer side has a protection against moisture. Each layer may therefore have two sides with different treatments and/or coatings. Alternatively or additionally, a printing and/or finishing is conceivable as a pretreatment. It is further conceivable that the at least one layer is subjected to aftertreatment after production. As an aftertreatment, for example, a coating, a printing and/or a lamination are/is conceivable. Alternatively or additionally, deformation and/or embossing and/or compaction are/is conceivable as an aftertreatment, such as in particular by pressure and heat, for example for a gripping geometry.

The handle body may be realized as a rod or as a tube. Herein the one or several layers of paper material are rolled. In an implementation as a rod, the handle body is completely rolled, such that the body is completely filled and there is in principle no cavity is formed in the body. In an implementation as a tube, one or several layers of paper material are rolled in such a manner that they form around a cavity. In this way a tube is then formed. The layers are connected to one another in both cases in such a manner that they hold stably to one another and form a stable handle body which can then be used further. Herein the application unit may be fastened to the handle body in the various ways described, inter alia by overmolding.

Optionally it would be conceivable that at least a portion of the surface of the body care product is subjected to aftertreatment. Possible methods are printing, immersion, spraying, etc. Herein it would be conceivable that the body care product is immersed, respectively varnished, for lower water absorption, or that the body care product is printed. Herein an advantageous water resistance is in particular expedient, since the application unit consists of paper. Body care products may also be applied. Of course, other methods described in this document for finishing, for the use of fillers or treatments of the paper material may also be used.

The aftertreatment may also include the at least partial application of body care agents, in particular dental care agents such as for example toothpaste, to the application unit. Furthermore, the aftertreatment may also create a surface of the application unit that is optimized for cleaning, for example by the application of abrasives. For a fixing of abrasives, adhesive substances suitable for oral hygiene are used.

The aftertreatment may take place in particular by immersing a respective portion of the product in a fluid.

Furthermore, it would be conceivable that the material handle body of the handle unit comprises at least two layers of a paper material which are connected to one another in a layered manner. For this purpose, individual layers may be prepared as separate elements and/or may be created by a bending of layers. This in particular advantageously allows producing a handle body. It is in particular possible to provide an advantageously stable handle body which is easily manufactured. In particular, an advantageous handle shape can be provided. Preferably, the material handle body, in particular the entire handle unit, consists of at least one layer. Alternatively, if there are at least two layers, these are preferably arranged at least partially one above the other and connected to one another. Preferably, 2 to 6 layers, preferentially 2 to 4 layers, are provided. However, it is also possible that 7 to 15 layers, preferably 8 to 12 layers, are provided. The layer thickness depends on the number of lays, thus the number of layers also depends on this in order to optimize the stability. The layers may consist of different paper materials and may have different densities and/or grammages of paper materials. The paper material/s may comprise additional layers made of films, in particular plastic films. Preferably the at least two layers are arranged, perpendicular to a main extension plane of the layers, at least partially one above the other and are connected to one another. The at least two layers in particular adjoin one another with a largest surface area. The layers may be connected to one another, for example, by gluing and/or by sealing and/or by pressing and/or by welding and/or by lamination. Pressure and/or heat and/or water (steam) is used. The layers are connected to one another in particular during a production of the body care product. During the connecting of the layers, the application unit can be fixed between the layers.

The connection and/or sealing of layers may be carried out applying various parameters. The preferred parameters during the sealing of layers are as listed below. The temperature varies in the range from 150° C. to 210° C. The pressure is between 4,000 N/m² and 6,000 N/m², preferably between 4,400 N/m² and 5,100 N/m². The sealing time is between 0.5 seconds and 5 seconds, preferably between 1.5 seconds and 3.5 seconds.

Furthermore, during the sealing, the sealing punch may for example be realized in a non-planar manner. In this case a continuous or non-continuous surface and/or a structure of the punch may be provided. Such a structure, in interaction with the counter-surface, allows creating a sealing structure that is sealed with different strengths or also with different characteristics. It is thus also possible to produce a pressure progression over the sealing surface, with the pressure varying over different regions of the material handle body of the handle unit.

During the sealing of several layers, it is furthermore possible to define a sealing sequence. For example, not all layers are sealed with one another at the same time. If for example three layers are sealed, the first two layers may be sealed first and then the third layer is sealed to the other two layers. In the case of four layers, for example the first two layers may be sealed and then either respectively one layer may be sealed in two sealing processes or the two further layers may be sealed in one sealing process. An optimal sealing of the individual layers is achievable using step-by-step sealing. In the step-by-step sealing, it is also possible that the sealing parameters such as temperature, pressure and time are adapted individually for each sealing operation.

Preferably the material handle body is layered from a sheet, from a roll or from a page layout. It is also conceivable that the at least two layers are pretreated, embossed, printed or finished before a processing operation (in this regard, see the further explanations in this document). The pretreatment, such as e. g. embossing, printing and finishing, may also concern only one layer. As a pretreatment, for example, a deformation and/or embossing and/or compaction is also conceivable, with the aim of producing a structure and/or a reinforcing flange, respectively a reinforcing rib, on the surface of the final product. Furthermore, as has been described, cavities may also be formed between the layers of the paper material/paper materials by deformation and/or embossing and/or compaction of the paper material. Herein, e. g. in the case of two layers, before the connection of the paper material layers, two opposed embossings, respectively deformations, are provided at the same position of the handle body in order to produce a handle body that is at least in sub-regions 3-dimensional instead of planar. Instead of a cavity, it is also possible with the described technique to anticipate a zone, respectively a recess, for a connection unit and/or application unit to be mounted between the provided layers later on. Alternatively, instead of two embossings, it is also possible to realize only one embossing, such that the cavity is smaller and the deformation is visible only on one layer.

The deformation and/or embossing and/or compaction of the handle body is possible, as has been discussed, with a single layer or also after the connection of several layers. In this way, it is likewise possible to apply surface structures or a flange for reinforcement. With this, a cavity can be produced no longer. However, it is likewise possible to form a one-sided zone, respectively a depression, respectively a recess, for a connection unit. The deformation is then in each case convex on one side and concave on the other side.

Alternatively or additionally, a coating is conceivable as a pretreatment. For example, an inner side of each layer of the planar paper material could be configured for an adhesive bonding while an outer side has a protection against moisture. Each layer may therefore have two sides with different coatings and/or properties. Alternatively or additionally, a printing is conceivable as a pretreatment. It is further conceivable that the at least two layers of the planar paper material are subjected to aftertreatment after the connection. As an aftertreatment, for example, a coating, a varnishing, an immersion, a printing and/or a lamination are/is conceivable. Preferably, the connection unit, respectively the application unit, is connected directly to the material handle body. The connection unit, respectively the application unit, may in particular be laid between the layers of the material handle body and may be connected together with the layers of paper material. The application unit is in particular quasi clamped between the layers that are to be connected. During the connection of the two planar paper material layers, the overlapping portion of the connection unit, respectively of the application unit, at least partly displaces the paper material. In this way, the hold of the connection unit, respectively of the application unit, is reinforced. The overlapping portion of the connection unit, respectively of the application unit, may be deformed in order to increase the hold in the paper material. As has been described above, a structure and/or a flange may also be produced in this portion by embossing in order to increase the hold of the application unit.

Alternatively, the connection unit, respectively the application unit, is applied onto the provided layer(s) on one side. Here, for example, welding, sealing, laminating, gluing, riveting or other connection techniques are suitable.

For a flosser, respectively a dental floss stick, the application unit may be clamped between the layers. In such a case the dental-floss thread is fixed between the layers of paper material. With the production at the same time the structure is described. A layer of paper material is provided. The layer is U-shaped in its front region. The dental-floss thread is placed on the two legs of the U-shaped geometry and tensioned. Then glue is applied (for example point-wise) on both legs on the dental-floss thread and on the paper before a further layer of paper material is placed thereon. In this way the paper material and the dental-floss thread are fixed to one another. In order to make the whole thing more stable for a utilization as a flosser, a further glue point may then be respectively applied at the outer exit points of the dental-floss thread from the brought-together layers of paper material. The dental-floss thread protrudes at that moment on both outer sides of the leg of the U-shaped geometry. The protruding length is cut to 1 mm to 10 mm, preferably to 2 mm to 6 mm. After this the loose thread ends can be melted, such that at the end a ball and/or a mushroom made of dental floss material is formed which adjoins the paper material and/or the adhesive/glue. In this way kind of an abutment is created when the flosser is used. The dental-floss thread is introduced between the teeth and is moved in the interdental space, which brings about a certain tension on the thread. The described fixing between the layers and/or at the edge of the layers of paper material by means of adhesive and/or of the material mushroom as an abutment enables good holding; the dental-floss thread is not torn out, respectively is not too much deformed between the legs of the U-shaped geometry.

Alternatively the application unit, i.e. the dental-floss thread, can be pretreated. In an injection molding tool holding elements, e. g. in the form of cylinders or balls, are injected onto the dental-floss thread. The balls are made of plastic, preferably a thermoplastic. The balls for an application unit lie apart by such a distance that they later get to lie in the region of the legs of the U-shaped geometry. The balls may form a connection unit. The layers of paper material are prepared in parallel and are in the region of the legs provided with a through hole, respectively a depression, in the size of the diameter of the injected balls. At least one of the layers is then provided with adhesive, the dental-floss thread plus ball is laid in, respectively positioned, such that respectively one ball gets to lie in one leg, respectively in its through hole, respectively its depression. The second layer is then placed onto the first layer and the thread is clamped in-between. The through holes, respectively depressions, of this layer likewise lie in the region of the balls, such that the balls are clamped between the layers. Alternatively, the layers may be connected by means of sealing or ultrasonic welding; it is likewise possible to melt the balls to a certain degree and to make the connection to the paper material even stronger. The ball diameter is equivalent to a size of 0.5 mm to 5 mm, preferably of 1.5 mm to 3.5 mm. The through hole, respectively the depression, accordingly likewise has a diameter in the same range.

As described above, on both outer sides of the balls the dental-floss thread may be cut to 1 mm to 10 mm, preferably to 2 mm to 6 mm. Subsequently the loose thread ends can be melted, such that at the end a ball, respectively a mushroom, made of dental floss material is formed adjoining the balls. This likewise leads to improved fixation of the dental-floss thread, in particular during use.

The pull-out weight for the thread in the case of the mentioned flossers is preferably more than 5 kg, preferably more than 7 kg, most preferably more than 8 kg.

In particular, an option is conceivable in which the number of layers of paper material varies. For example, in the region of the anchoring, where a specific stability must be achieved, there may be more layers than on the remaining handle—or vice versa. Not every layer must necessarily extend over the entire region of the adjoining layer. A reduced layer may lie on the outside or may be arranged between larger layers. The arrangement between the layers allows avoiding damage due to tearing away. In particular, there are no interfering outside-situated edges from which a risk of injury may arise.

As already described, the layers may in general be molded from the same sheet or page layout in that they are folded and then sealed. As a result, hinge-like folding edges are formed. The folded layer may be realized identically to the non-folded layer of the sheet or of the page layout but may as also be smaller than the non-folded layer of the sheet or of the page layout.

With a crease or a perforation, the folding can be simplified and/or the lay may be defined. Preferably, however, the outer side of the fold remains undamaged. Preferably, the crease or perforation is arranged on the inner side of the fold. Folded layers have the advantage that fewer cut edges of the paper material are exposed to moisture and as a result the water absorption is reduced in this location.

A further possibility of protecting the cut edges of the paper material is the flanging. Individual layers or several layers of the cut edge may be at least partly flanged in the peripheral region. This provides the body care product with additional stability and reduces the risk of injury, in particular when used in the oral cavity. A further possibility is a subsequent machining of the cut edges. Herein the sharp edges are rounded as much as possible. This may be done using removing methods (e. g. cutting, grinding, etc.) and/or by compacting.

The compacting of edges in order to round them preferably takes place after the cutting. The cutting/punching of paper material entails the edge being rounded on the start side (entry side) of the cut while the edge on the end side (exit side) of the cut is sharp. The immersion of the knife into the paper material entails a rounding. In order to round the opposite sharp edge, the respective side is treated with a punch. The edge is compressed by a punch with a round contour, such that the sharp edge disappears. In this way it is achievable that a product consisting of only one layer of paper material has rounded edges on both sides. As a result, in the edge region higher density is achieved in the volume. Possibly it is necessary during the compacting to support the counter-contour by a support contour, for example such that the already round edge is not deformed but adjoins the support contour. The support contour further allows even more precise shaping of the supported contour, for example such that the layer which is pressed is more precisely pressed into the support contour and the contour is thus formed more precisely.

When using rounded edges on products consisting of several layers, it should be noted for the user's ergonomics that the rounded edges are preferably situated on the outside. Herein the described post-processing by the pressing punch may be omitted if the punched layers are layered in such a way that the respective entry sides of the punching are situated towards the outside, because as described they are already rounded on account of the method. The outer edges are thus rounded in the final product.

It is further proposed that the connection unit comprises at least one receiving groove configured to receive a front edge of the material handle body of the handle unit, which faces towards the application unit. The receiving groove is in particular configured for a form-fitting and/or clamping accommodation at least of an edge of the material handle body. The receiving groove preferably extends in particular at least substantially perpendicular to a longitudinal axis of the handle unit. If several receiving grooves are used, they are preferably arranged at an angle to each other, wherein the receiving grooves are thereby still arranged symmetrically with respect to the longitudinal axis. In particular, the receiving groove is preferably configured to receive a front edge of the material handle body. A possible arrangement at an angle parallel to the receiving feature also enables an orientation of the handle unit relative to the connection unit. It is conceivable that for example barbs in the form of depressions, recesses, undercuts etc. are provided in the receiving groove, which improve a clamping. The receiving groove is in particular configured to provide a clamping connection with the handle unit, in particular with the material handle body. In a clamping connection, the handle unit is in particular clamped in a receptacle, in particular the receiving groove, of the connection unit. The receptacle is herein preferably made of plastic, such that a plastic of the connection unit is arranged on the outside, with the connection unit receiving a paper material of the handle unit on the inside. The paper material may herein for example be pushed into a U-shaped element of the connection unit, which delimits the receiving groove. The clamping connection may in particular be advantageously combined with further connection possibilities mentioned in this document, such that deployment out of the receptacle can be prevented. The term “substantially perpendicular” is here in particular to define an orientation of a direction relative to a reference direction, wherein the direction and the reference direction, in particular when viewed in a projection plane, include an angle of 90° and the angle has a maximum deviation of in particular smaller than 8°, advantageously smaller than 5° and particularly advantageously smaller than 2°. This in particular allows providing a connection which is particularly easy to produce.

Beyond this it is proposed that the material handle body of the handle unit forms at least one stabilizing wall, which is angled with respect to a main extension plane of the handle unit and extends at least substantially parallel to a longitudinal axis of the handle unit. The stabilizing wall is preferably arranged at an outer edge of the material handle body, which extends substantially parallel to the longitudinal axis. The stabilizing wall is, perpendicular to the longitudinal axis of the handle unit, angled with respect to a main extension plane of the handle body, in particular at an angle of at least 10°, preferably at least 20° and particularly preferably at least 30°. The stabilizing wall is, perpendicular to the longitudinal axis of the handle unit, angled with respect to a main extension plane of the handle body, in particular at an angle of less than 60°, preferably less than 45° and particularly preferably less than 35°. Preferably the stabilizing wall forms a free outer edge of the material handle body. However, it would also be conceivable for the stabilizing wall to be formed centrally in the material handle body. Beyond this, further stabilizing structures may be provided in the material handle body. The stabilizing structures may preferably be realized predominantly in the longitudinal direction, in particular for improving the stability of the handle body and/or for better receiving the forces during the intended use. The stabilizing wall may moreover form at least one holding surface. For this purpose the stabilizing wall may be provided with structures. The at least one holding surface may be formed laterally on the handle body. The holding surfaces are in particular configured to facilitate holding. In the region of the stabilizing wall, the surfaces are in particular deeper in cross-section than the cross-section of the handle body is in another point. In the region of the stabilizing wall, the material handle body preferably has a substantially U-shaped or V-shaped cross section perpendicular to the longitudinal axis of the handle unit, preferably a cross section with an opening towards the rear or front. This in particular allows providing an advantageously stable handle unit.

It is further proposed that the connection unit comprises at least one delimiting wall, which is configured for an at least partial delimitation and stabilization of the stabilizing wall of the material handle body. The delimiting wall in particular serves for holding the stabilizing wall of the handle unit in position. The delimiting wall in particular extends at least substantially parallel to a longitudinal axis of the handle unit. The delimiting wall is preferably arranged on an outer edge of the connection unit, which extends substantially parallel to the longitudinal axis. The holding surfaces on the handle unit may continue into holding surfaces on the delimiting wall of the connection unit. The delimiting wall on the connection unit may serve as a support for the stabilizing wall of the handle unit. It is here for example conceivable that the stabilizing wall of the handle unit adjoins the delimiting wall of the connection unit on its inner side. Alternatively, it would be conceivable that the stabilizing wall of the handle unit is inserted into a geometry directly at the delimiting wall of the connection unit. Of course a fix connection between the delimiting wall of the connection unit and the stabilizing wall of the handle unit may also be created with assisting means, for example by gluing, by sealing, by welding, by clamping, by latching, by riveting, by overmolding, and/or by other connection methods mentioned in this document, etc. This in particular allows providing an advantageously stable handle unit. It is moreover possible to provide an advantageously high level of ergonomics. In particular, reliable gripping of the body care product is enabled.

The connection unit may preferably comprise holding points, support points or contact surfaces, which support the user of the product in the application. Thus for example in the case of toothbrushes or brushes, finger rests or thumb rests may be provided. For this purpose, the connection unit may be expanded lengthwise along the handle unit, wherein the expanded portion forms a support, in particular a thumb rest. Alternatively, in the case of a razor, the above-mentioned holding surfaces may be provided, which provide additional gripping surfaces laterally on the narrow side of the handle unit.

Furthermore, the connection unit may comprise one or several support elements like the delimiting wall, which are realized as a portion of the connection unit. The bent handle body may here correspondingly adjoin the support elements, in particular a corresponding delimiting wall. Herein the support elements provide support and ensure that the handle is not bent open. In this way the structure of the handle body can be held in shape. Herein the support elements, in particular the delimiting wall, may in particular be combined with holding points, support points or contact surfaces, such that for example the support element may serve as a support element on the inside while serving for holding on the outside.

With regard to the contact surface of the handle body on the connection element, the delimiting wall may be arranged centrally or eccentrically in the direction of the height axis. This means that the delimiting wall may project beyond the handle body on the front side as well as on the rear side.

The delimiting wall may also be equipped with form-fitting elements. The form-fitting elements may be constructed as latching elements. These form-fitting elements are arranged on the front side of the connection unit and are realized as projecting elements, preferably as projecting edges in the direction of the width axis. The elements are attached on both delimiting walls and are directed towards one another, respectively directed towards the handle unit. In the assembled state, they clamp the handle unit, respectively the handle body, with respect to the connection unit. The form-fitting elements herein press the handle body against the contact surface of the base body of the application unit at the connection unit. The latching elements are preferably configured to be elastically deflected during a fastening process in order to then latch, due to an inner tensile force, behind an edge of the handle unit. The advantage of this design of the connection between the handle unit and the connection unit is that the handle does not have to be provided with a through hole by additional punching. The punching is realized merely along the outer contour.

Furthermore, holding elements in combinations of handle unit and connection unit are also possible. It is thus possible, for example, to obtain a holding element in the combination of elements of the handle unit and of the connection unit. It would be conceivable, for example, that the form-fitting element of the connection unit is realized in a mushroom shape and extends through a pass-through recess in the handle unit, wherein the mushroom-shaped form-fitting element is formed such that the end projecting freely through the handle unit can also be used, for example, as a holding element and/or as a thumb rest. Alternatively, it would be conceivable that the form-fitting element is realized as a ring latching on a pass-through recess laterally, such that the pass-through recess remains partly open. The pass-through recess can then be used together with the annular form-fitting element as a thumb rest.

Beyond this it is proposed that the material handle body of the handle unit is formed in substantially S-shaped curved fashion along a longitudinal direction of the handle unit. Preferably the material handle body is realized such that it is deformed relative to a planar design. Preferably the material handle body has an S-shaped longitudinal form in a longitudinal section and/or in a longitudinal view. The profile of the handle unit is optimized in particular for an application and for user ergonomics. The material handle body in particular has a stepless continuous rounded shape in the longitudinal direction. Particularly preferentially the material handle body has an S-shaped curved shape in the longitudinal direction. This in particular allows providing an advantageously ergonomic and stable handle unit.

Furthermore, in a cross section in the transverse direction, the material handle body of the handle unit in particular has an advantageously shaped transverse shape. The transverse shape may in particular generally have structures which have a positive influence on stability and continue in a stepless manner in the longitudinal direction. Moreover, it would be conceivable that shapes may merge into one another in the longitudinal direction. Preferentially, the material handle body is in particular realized so as to be symmetrical in a plane parallel to the longitudinal axis and perpendicular to the transverse axis. Preferably the shapes of the transverse shape extend from bottom to top preferably into the neck region to the connection unit. Exemplary cross-sectional shapes may for example be a U-shape, a T-shape, an Ω-shape, a W-shape, a V-shape, an L-shape or also a combination, for example of several shapes in the longitudinal direction. The corners of these transverse shapes, respectively the longitudinal edges resulting therefrom, are preferably rounded, as has been described before. The material handle body may for example have an undulating cross-sectional shape. Furthermore, several ring segments may be combined to form a cross-section shape. In a plan view, in particular any shapes are conceivable for the material handle body which are adapted to the application. Thus, for example, a rectangular, a square, an oval, a round and/or a triangular shape are/is conceivable for the material handle body in a plan view. The mentioned shape elements may also be combined in the longitudinal direction. In particular, stability can be improved by the shape of the handle body, such that the forces resulting from a load on the application unit can be received. The handle unit is in particular capable of elastically receiving forces up to a load on the application unit of 1,500 g, preferably up to 2,000 g. Herein the application unit is fixed and the force is measured at the rear end of the handle unit—herein the handle unit must not bend and must be essentially able to elastically reassume its original shape.

Preferably the handle unit, in particular the material handle body, may comprise a suspension recess. The suspension recess is in particular designed in such a manner that in a utilization it does not interfere with the gripping of the handle unit and can be used as a feature for suspending the product. The suspension recess is preferably designed such that it is situated at the handle end that faces away from the application unit. It would also be conceivable that the handle unit, in particular the material handle body, comprises an element for putting over that can be pushed out; herein the element may be designed in such a manner that it can be partly pushed out of a basic shape of the handle body. For this purpose in particular a structure is created that is to be broken or already broken, i.e. already punched. Herein it is in particular possible that just a line is punched in or that a surface, in particular a crescent-shaped surface, is punched out which partially exposes the element. The structure that is to be broken may in particular be realized for example by a perforation or by a different weakening, such that the element can be pushed out manually. By means of the element the product may for example be suspended on a toothbrush glass.

Preferably the material handle body of the handle unit comprises at least one layer of a paper material, the layer having an embossing and/or a deformation. During the embossing the paper material is at least partly deformed by pressure. In this way a three-dimensional design and/or a surface structure are/is achievable on the surface of the paper material in sub-regions of the surface. It is also possible to obtain cavities, respectively recesses, within the layers of paper materials by embossing and/or compaction. In this way improved stability and/or grippability, respectively slip resistance, of the handle body is achieved and thus the handling of the oral hygiene product is facilitated. In principle, this enables embossing and/or compaction of all paper materials, as well as alternatively also wood and bamboo, be they in a planar, rolled, laminated, sealed and/or welded state or the like. An embossing, respectively a compaction, may be aided by an embossing tool (negative form) using pressure and/or heat and/or water (steam), etc. An embossing, respectively compaction, can be made in the planar state during the punching-out of the paper material. However, it is also possible that an embossing is carried out on an already-rolled paper material. The embossing, respectively compaction, may also be a manner of deep-drawing, which likewise entails the above-mentioned design possibilities. The deep-drawing of the paper material may be aided by a deep-drawing tool, likewise using pressure and/or heat and/or water (steam), etc. An embossing, respectively deep-drawing, may also give the paper material a shape providing stability, for example a depression in the longitudinal direction of the handle portion of a toothbrush, which makes the handle body more stable. Furthermore, specifically selected coatings, such as varnishes etc., may generally influence and also improve dimensional stability and stability in general.

It would furthermore be conceivable that the body care product, in particular the material handle body, comprises at least one layer, which is at least partly deformed and/or pressed and/or deep-drawn. The at least one layer may in particular be produced from one or more sheets or page layouts of a paper material and/or rolled from rolls. The body care product in particular comprises several layers. The layers, in particular of the material handle body, may be connected to one another for example by gluing, by welding and/or by lamination. It is also conceivable that the at least one layer is pretreated before a processing operation. As a pretreatment for example embossing is conceivable, with the aim of creating a structure on the surface of the final product. Alternatively or additionally, a coating is conceivable as a pretreatment. For example, an inner side of each layer could be configured for adhesive bonding while an outer side has a protection against moisture. Each layer may therefore have two sides with different coatings. Alternatively or additionally, a printing is conceivable as a pretreatment. It is furthermore conceivable that the at least one layer is subjected to aftertreatment after production. As an aftertreatment, for example, a coating, a printing and/or a lamination are/is conceivable. However, correspondingly an alternative general basic shape for a handle unit is also conceivable. The at least one layer of the body care product is in particular deformed, preferably deep-drawn, in particular using pressure and heat. This in particular creates for example a gripping geometry for improved ergonomics. It is in particular possible to provide a body care product with a voluminous handle unit. It would also be conceivable that the handle unit is made up of two deep-drawn half shells which, in a state when connected to one another, enclose a hollow space. The connection of the half shells may be realized, for example, by gluing, welding, sealing and/or plugging. It is thus in particular possible, for example, to produce the handle unit of a toothbrush. Layer thicknesses of the planar paper material are 0.5 mm to 3.0 mm, preferably 0.7 mm to 2.5 mm, particularly preferentially 1.0 mm to 2.0 mm. Alternatively to the layer of paper material, a layer of planar wood material or bamboo material may also be made use of. Layer thicknesses of the planar wood material, respectively bamboo material, are 0.3 mm to 3.0 mm, preferably 0.5 mm to 2.0 mm, particularly preferably 0.7 mm to 1.8 mm. As already explained, one, two or more layers of the materials may be brought together to form a handle unit.

It is also conceivable that means such as separating aids and/or predetermined breaking points are provided on the body care product, at which the personal care product may be dismantled for disposal or may be separated with or without simple aids (such as, for example, scissors, knives, screwdrivers etc.), such that the various components, respectively materials, may be separated at least partially in a variety-specific manner for disposal purposes. For example, between layers to be separated, perforations, removable projections gripping tabs may be realized by means of which the materials, respectively layers, can be separated. Alternatively or additionally, non-connected, non-sealed or non-welded sections may be configured for a separation. Furthermore, auxiliary perforations and/or creases and/or notches may be provided, for example for separating the application unit and/or the connection unit from the handle unit, i. e. for example a separation of brush and paper. Preferably, in particular separating aids, such as in particular pull tabs, gripping tabs and/or perforations, are provided on the body care product.

Preferably on the connection unit and/or on the application unit means, respectively portions or sub-regions, may be formed which enable and/or indicate and/or facilitate a separation of the connection element, which is preferably made of plastic, from the handle unit, which is preferably made of a paper material. This in particular enables separate disposal. The connection unit may in turn be realized with the application unit in an inseparable manner. In the case of a razor, accordingly the connection unit with the application unit must then be disposed of in the waste. However, it would also be conceivable that a blade is realized so as to be separable from plastic parts, such that the plastic parts can be supplied to a plastic collection. This enables at least partial recycling. For example, a tab, edge, projection, etc., which can be easily gripped but does not interfere with normal use, may be provided on the connection unit. Preferably, the tab can be pulled and the handle unit can be moved in the opposite direction in order to tear apart the parts, in particular the handle unit and the connection unit.

The detachment of sub-regions, respectively elements, of the body care product, respectively the at least partial separation of materials (such as, for example, separating the application unit from the handle portion; separating the injection-molded bristles and/or the bristle platelet from the head portion) is provided only once (e. g. for the separate disposal of materials). Preferably a reconnection of the separated elements is intended.

It would moreover be conceivable that the material handle body of the handle unit comprises at least one layer of a paper material which is realized in at least partly folded fashion. This in particular advantageously allows producing a handle body. It is in particular possible to provide an advantageously stable handle body which is easy to produce. In particular, an advantageous handle shape can be provided.

Preferably, the layer is produced from a coherent, in particular cut-to-size, paper and/or cardboard. The layer advantageously has at least one folding axis and/or at least one folding edge. In a usage state, the layer is preferably folded at the at least one folding axis and/or the at least one folding edge. Advantageously, by the at least one folding axis and/or folding edge, folding of the layer into a usage state is facilitated or the product obtains only by the folding its shape that is necessary to enable use. It would be conceivable that the layer is realized in the folding edge and/or folding axis in perforated and/or creased and/or embossed fashion in order to enable defined folding.

Preferably, the layer is divided by the at least one folding axis into two sides, in particular sub-layers. Preferably the at least one folding axis and/or folding edge runs parallel to the main extension direction of the body care product. It is also conceivable that at least one folding axis runs perpendicular to the main extension direction of the body care product. The folding edge may be realized in a continuous or non-continuous manner; furthermore a combination of perforations, creases, cuts may form the folding edge. The elements may for example be arranged one behind the other. The folding edge may be realized in a straight or curved fashion. This allows forming shapes which are waisted when the body has been assembled. The material handle body may herein in particular be folded from paper sheets or from a roll. Furthermore, a connection of folding sides may in particular be brought about by sealing or gluing or mounting (such as plugging, latching). If the folding sides are plugged or latched into one another, for example notches, which can be slid into one another during assembly, are realized on the corresponding surfaces. A plug-in tab is then formed between notches on the same surfaces. If the notches are slid into one another, the plug-in tabs slide one above the other, such that in the final stage one plug-in tab lies over the other, i.e., above the other.

By the folding of the sides, in particular sub-layers, a body can be shaped which forms a closed, in particular triangular, cross section perpendicular to the longitudinal axis. The triangle may herein be implemented in an equilateral or isosceles manner, wherein preferably a symmetry with respect to the vertical axis is created. The orientation of the folding axes defines the way in which the surfaces are oriented. The folding axes may in each case be parallel or tapering. If the folding axes are designed to be tapering, the place in which the distance between the folding axes is the smallest is preferably on the side of the brush head. As a result, a body can be formed which has an advantageously high level of stability in the region of the application unit. The front side may be formed, for example, by folding axes which run in parallel, while the folding axis on the rear side is designed to be tapering to the folding axes of the front side, such that a conical shape is formed in the side view. By the folding the sub-layers are placed at least partly one above the other and, in the final product, at least certain sides of the product are in double fashion, such that a plurality of sub-layers lie one above the other on defined sides. This means that several, in particular two, sub-layers lie one above the other on at least one side. By applying several layers, the body obtains better stability. The connection of the layers may take place as described; for example, they may as well be glued or sealed to one another.

The cut edges present a challenge on the application side. For example when used as a razor or toothbrush in a moist environment, the possibly untreated cut edges are open places of the layers, which may take in water. In order to form better protection and also to better ensure long service life, the application unit, in particular the base body of the application unit, may be designed with the connection unit in such a manner that it comprises the end of the folded body on the application side, which means that the folded body is enclosed in thimble-like fashion by the application unit with the connection unit. Herein the base body of the application unit and/or the connection unit are in particular made of a water-resistant material. The application unit, in particular the base body of the application unit, with the connection unit is preferably put over the folded material handle body. In this way better protection of the open cut edges of the layers is achievable.

The folded body may be used for a wide variety of body care products, in particular for toothbrushes or razors.

As a pretreatment, for example an embossing and/or compaction is conceivable, with the aim of producing a structure on the surface of the final product. Alternatively or additionally, a coating is conceivable as a pretreatment. For example, an inner side of a layer could be configured for adhesive bonding while an outer side has a protection against moisture. The layer may therefore have two sides with different pretreatments, such as for example coatings. Alternatively or additionally, a printing is conceivable as a pretreatment. It is furthermore conceivable that the at least one layer is subjected to aftertreatment after production. As an aftertreatment, for example, a coating, a printing and/or a lamination are/is conceivable. Preferably the layer of the material handle body is cut or punched.

As a body care product with a corresponding material handle body, in particular a toothbrush would be conceivable. The body care product is in particular produced in a flat manner, wherein in particular creases, embossings, perforations or the like are provided for a folding prior to first use. The creases are in particular arranged in the folding axis. In addition, a combination of folding and subsequent connecting, such as, for example, by adhesive bonding, by sealing, by welding, by gluing, by laminating, would be conceivable. In the case of a body care product realized as a toothbrush, the body care product could realize an origami-style framework made of paper. Alternatively or additionally, in an implementation as a toothbrush, the body care product could comprise cleaning elements made of a paper material. The cleaning elements could be arranged on a bristle platelet made of plastic, of wood or of a paper material, the bristle platelet being arranged and/or fixed on the paper material of the handle body.

The body care product is in particular produced from a planar paper material, wherein in particular creases are provided for a folding prior to first use. The creases are in particular arranged in the folding axis. For use, the body care product may in particular be folded to form a triangle, respectively to form a pyramid-shaped or cone-shaped structure. Optionally, in the case of a folded body care product, a framework made of paper material could also be provided on the inside while a casing surrounds the framework on the outside.

As a further body care product with a corresponding material handle body, a toothbrush would be conceivable (analogously, these possibilities are also available, inter alia, for other body care products such as wet shavers). The paper material is prepared as a layer with a respective creasing, perforation or the like, such that a corresponding product can be folded subsequently. The toothbrush produced from a paper material in this way may be created with a variety of cross-sectional profiles. For example, in a usage state, the product may form a closed profile, e. g. a triangular, a tetragonal or generally an n-gonal profile; an open profile, e. g. a U-profile, V-profile, a W-profile, T-profile, L-profile; or a profile combined therefrom. The profile eventually forms a body which implements the handle body of the toothbrush.

The profile may herein also serve to form, for example, a standing surface on the rear side of the toothbrush, such that the toothbrush can be placed on a surface and then does not roll around its own axis. The creasings, perforations or the like prepared for the foldings do not necessarily have to be extended over the entire body; they may also end in a surface such that then, for example, a rounding results. The assembly of the product may take place in various ways. For example, the product may already have been completely assembled or the end customer (user) assembles the product after purchase, respectively before use. The second variant may provide various advantages with respect to the space required in the packaging since the body care product is in the flat state less voluminous than in an assembled state. During assembly, different zones of the layer are connected to one another in order to form the voluminous body, respectively the handle portion. The layers may be connected directly by adhesive strips, by glue or by another means suitable for the connection. The bristle field may already have been mounted on the handle or it may likewise be fixed with the assembly of the handle, for example by gluing. Prior to assembly, it may also be possible to separate off certain places of the layer of paper material. Beyond this, it is possible that elements are realized which enable the body of the product to form a closed or semi-open hollow space in a ready-to-use state. Thus, certain tabs are formed which are plugged into openings and fixed therein in such a way that the openings are then closed. The foldings may be realized in different directions; preferably, the majority of the foldings are made around folding axes in the longitudinal direction. However, foldings in transverse directions may also be prepared, for example if elements are folded into one another for packaging. Furthermore, elements may be provided which are to be folded in such a way that, for example, the handle body is closed on the side of the application unit (e. g. bristle field). Tabs of this kind are moreover realized in such a way that they are placed in a certain range around sub-layers. Herein they bring about a further stabilization of the sub-layers opposite one another.

It is also possible to provide the layers made of paper material in the flat state (before the folding) with structures or other surface coatings etc. in order to create surface elements in the final product which fulfil specific properties, e. g. have higher friction.

The material handle body may be provided with a surface structure for improving the haptics, respectively the holding. Thus, for example, nubs may be provided on a surface of the handle body, which are shaped with the embossing of the remaining body. The surface structure may be integrated in various regions of the handle body, such as for example on the mentioned holding surfaces or on the further surfaces.

The embossing is realized as already described using pressure and/or heat. The paper layers may be somewhat moistened for this purpose, e. g. with water (steam), such that the forming is facilitated. In this way, by the treatment with heat, moisture and pressure, the product may assume a shape, which extends into a further dimension, e. g.: handle body with a bend, respectively an angle therein.

Alternatively or additionally, the connection section of the application unit may be realized as an interface for an exchangeable part, such as for example an exchangeable head for a manual toothbrush.

Optionally, the material handle body may already have been produced from a printed paper material. This would enable creating a colored final product without post-processing.

Furthermore, it is proposed that the material handle body of the handle unit comprises at least one layer of a paper material, which is realized in at least partly folded fashion, wherein the handle body has a storage position in which an inner side of the layer is directed outwards, and an operational position in which an outer side of the layer is directed outwards. Thus, the handle unit may in particular be folded inwards when packaged and folded outwards for use. Here it is in particular conceivable that the handle body is combined with a packaging. Preferably, in a presentation state the layer forms at least part of a packaging of the body care product. Preferably, the moving of the handle body from the storage position into the operational position is irreversible since certain elements may have to be separated off for moving from the storage position to the operational position.

Furthermore, it would be conceivable that the base body of the application unit comprises an injection-molded bristle field. In particular, directly injection-molded bristles are conceivable. These may in particular be produced together with the application unit, the handle unit and/or the coupling unit during a multi-component injection molding, or they may be subsequently injection-molded to a base body of the application unit. In contrast to conventional bristles, injection-molded bristles are not extruded but are molded via an injection-molding procedure. Special materials are used for this purpose. Injection-molded bristles may be directly injection-molded on the paper material or wood, i. e. the materials are laid into the injection-molding tool and are then overmolded.

It would also be conceivable that the body care product is at least partly milled from a paper material. The body care product is herein in particular at least partly produced from solid fiberboard, which is milled into a defined shape. It is herein in particular conceivable that the at least one solid fiberboard is subjected to aftertreatment after milling. The solid fiberboard may also be made of several layers of a paper material. As an aftertreatment, for example, a coating, a printing and/or a lamination are/is conceivable. Preferably in particular at least the handle unit is made of solid fiberboard. Different solid fiberboards, deemed expedient by someone skilled in the art, are conceivable, such as in particular Finnish paperboard. In this context, in particular a punched toothbrush would be conceivable as a body care product. For this purpose in particular a block of paper material is milled into a contour, wherein holes are drilled in the later brush head. In production, in particular in a first step the handle unit and parts of the application unit are milled and drilled from a block of paper material. Then the handle unit and/or the application unit may optionally be printed and varnished.

It would also be conceivable that the body care product is at least partly cast from a paper material. The paper material is in this case in particular processed in an injection-molding-like procedure. However, the processing of the paper material is realized at significantly lower temperatures and injection pressures. Respective methods are known, for example, from PurePaperPak, a constructional paper packaging. Herein in particular an ecological raw material use is possible, wherein the ingredients originate in nature and in particular consist by 100% of natural resources. The raw products are in particular composed of industrial starch, preferably of potatoes, paper fibers, water, including admixture. The resulting paper mixture is then used for further processing by injection molding technology. The paper injection molding is in particular realized in three steps. In a first step an injection is performed in which the paper mixture is injected into an aluminum tool. In a second step a baking process is carried out, in which the paper mixture is baked, in particular heated, in the tool. In a third step a demolding is carried out, in which the finished packaging can be removed. Furthermore, so-called PAPACKS® packagings are known as packagings. The packagings in particular consist of food-certified PAPACKS® fiber casting. Respective products are in particular compostable and can also be introduced into the waste paper circuit or bio-waste circuit. It is herein in particular conceivable that the cast body care product is subjected to aftertreatment after casting. As an aftertreatment, for example a coating, a printing and/or a lamination are/is conceivable. In this context, in particular a punched toothbrush would be conceivable as a body care product. For this purpose a toothbrush body, in particular the handle unit, is in particular cast from a paper material. In particular, in a production, the handle unit is cast from a paper material in a first step. Then the handle unit and/or the application unit may optionally be printed and varnished.

For injection molding with a paper material, in particular paper with starch (industrial starch) and a solvent may be used as materials. The components are mixed and are processed by suitable injection-molding machines. The materials are mixed in a ratio of 25% to 70%, preferably 30% to 40%, paper, 20% to 40%, preferably 25% to 35%, starch and 5% to 40%, preferably 10% to 35%, solvent. An industrial starch, for example of corn or potatoes, may be used as a starch. In particular, polyvinyl alcohol serves as a solvent. For mixing the components, these are dissolved in water, mixed and the resulting liquid/mass is dried and then processed into a granulate.

The paper material of the body care product, in particular of the handle unit, may also be injection-molded separately with an injection-molded part with at least one functional component, and may then be connected to the paper material in a downstream process. This would work for already mentioned examples (e. g. brush head with injection-molded bristles or bristle platelet with conventional/extruded filaments as toothbrush, interdental cleaner, flosser, etc.). Herein means for a connection to the paper material are provided on the at least one injection-molded part with at least one functional component. The connection is then realized by gluing, adhesive bonding, sealing, riveting, deformation, etc.

If one or several layers of paper material are used, as described in this document in other places, this/these may of course be processed, before or after the injection-molding and/or connecting, using various methods mentioned in this document (folded, deformed, embossed, creased, finished, coated, etc.).

As an alternative to a rolling of paper material, in particular an extrusion of paper material would also be conceivable for creating a cylindrical structure. As an alternative to a milling of paper material, in particular the twisting of paper material would also be conceivable for creating rotationally symmetrical geometries.

It is further possible to create a specific surface of the paper material. In particular, different surfaces may be created on different sides, for example with respect to coloring or surface quality.

According to the disclosure of the invention, a connection by welding does not have to take place over the entire contacting surface. It is thus for example possible that tear-open tabs and/or certain edge positions and/or edge zones are not welded. Furthermore, a welding may for example also take place only at the edge while inner surfaces are omitted. Furthermore, it would also be conceivable that weldings are omitted, for example so as to create mobility. Alternatively, it would also be conceivable that welding is deliberately carried out in order to create mobility, in particular in the region of a folding edge. Alternatively or additionally, welding may also be carried out only in the region of the interface to the application unit, for example only in a connection section of the base body.

Beyond this it is proposed that the paper material has a water-repellent coating and/or impregnation. A suitable coating and/or impregnation may also enable a reduction of the water absorption of the paper material. With this the paper material may also be suitable for multiple use. Herein an impregnation may be realized for example by means of a plastic and/or a wax or the like.

It is further proposed that the paper material is realized in varnished and/or printed fashion. In particular, advantageous individualization of the paper material is achievable in this way. In particular, advantageously simple printing is enabled on a paper material. In particular, finishing is achievable. A finishing may, for example, be a varnishing; here, for example, UV high-gloss varnish, structural varnish, soft-touch varnish or glitter varnish may be used. Alternatively or additionally, the paper material may have a printing. The paper material may, for example, have a printing with water-based varnishes, which are ecological compared to conventional varnishes.

Compared to conventionally produced body care products, the center of gravity of the entire body care product is displaced towards the application unit. In a production according to the invention, the handle unit produced from paper material has a lower weight, while a weight of the elements in the region of the application unit remains the same.

A center of gravity of the entire body care product in particular lies, measured from the upper end, in the range of 25% to 45%, preferably 30% to 40%, of the total length of the body care product. The total weight of the body care product is in particular from 0.5 g to 15 g, preferably from 1 g to 8 g. In an implementation of the body care product as a toothbrush, a weight is in particular 4 g to 15 g, preferably 5 g to 8 g. In an implementation of the body care product as a razor, a weight of the handle unit is in particular 5 g to 8 g, preferably 3 g to 12 g.

Furthermore, various packagings for the body care product, deemed expedient by someone skilled in the art, are conceivable. For example, conventional plastic blisters or packagings made of paper material are conceivable. In a packaging made of a paper material, in particular the use of plastic may be largely reduced.

The invention is furthermore based on a method for producing the body care product.

The body care product according to the invention shall herein not be limited to the above-described applications and implementations. In particular, in order to fulfil a functionality that is described here, the body care product according to the invention may comprise a number of individual elements, components and units that differs from a number given here and/or may comprise any expedient combination thereof. Moreover, regarding the value ranges indicated in this disclosure, values situated within the mentioned limits shall also be considered as being disclosed and as being usable as desired.

Of course, the implementation variants shown in this document are exemplary. Within the scope of the invention, the individual realizations and elements of these implementation variants may be combined with other implementation variants without departing from the scope of this invention.

### DRAWINGS

Further advantages will become apparent from the following description of the drawings. The drawings show six exemplary embodiments of the invention. The drawings, the description and the claims contain a plurality of features in combination. Someone skilled in the art will purposefully also consider the features individually and will find further expedient combinations.

In the drawings:

FIG. 1A shows a handle unit of a body care product according to the invention that is realized as a toothbrush, in a schematic 3D view from the front,

FIG. 1B shows the handle unit of the body care product according to the invention, in a schematic 3D view from the rear,

FIG. 1C shows the handle unit of the body care product according to the invention, in a plan view from the front,

FIG. 1D shows the handle unit of the body care product according to the invention, in a plan view from the rear,

FIG. 1E shows the handle unit of the body care product according to the invention, in a schematic sectional illustration along the section line A-A,

FIG. 1F shows an application unit and a connection unit of the body care product according to the invention, in a schematic 3D view from the front,

FIG. 1G shows the application unit and the connection unit of the body care product according to the invention, in a schematic 3D view from the rear,

FIG. 1H shows the application unit and the connection unit of the body care product according to the invention, in a schematic sectional illustration along the section line B-B,

FIG. 1I shows the body care product according to the invention with the handle unit, the application unit and the connection unit, in a schematic 3D view from the front,

FIG. 1J shows the body care product according to the invention with the handle unit, the application unit and the connection unit, in a schematic 3D view from the rear,

FIG. 1K shows the body care product according to the invention with the handle unit, the application unit and the connection unit, in a schematic sectional illustration along the section line C-C,

FIG. 2A shows a handle unit of an alternative body care product according to the invention that is realized as a toothbrush, in particular a travel toothbrush, in a schematic 3D view from the front,

FIG. 2B shows the handle unit of the alternative body care product according to the invention, in a schematic 3D view from the rear,

FIG. 2C shows the handle unit of the alternative body care product according to the invention, in a plan view from the front,

FIG. 2D shows the handle unit of the alternative body care product according to the invention, in a plan view from the rear,

FIG. 2E shows the handle unit of the alternative body care product according to the invention, in a schematic sectional illustration along the section line D-D,

FIG. 2F shows an application unit and a connection unit of the alternative body care product according to the invention, in a schematic 3D view from the front,

FIG. 2G shows the application unit and the connection unit of the alternative body care product according to the invention, in a schematic 3D view from the rear,

FIG. 2H shows the alternative body care product according to the invention with the handle unit, the application unit and the connection unit, in a schematic 3D view from the front,

FIG. 2I shows the alternative body care product according to the invention with the handle unit, the application unit and the connection unit, in a schematic 3D view from the rear,

FIG. 2J shows the alternative body care product according to the invention with the handle unit, the application unit and the connection unit, in a schematic sectional illustration along the section line E-E,

FIG. 3A shows a further alternative body care product according to the invention with a handle unit in a first alternative implementation, with an application unit and a connection unit, in a schematic 3D view from the front,

FIG. 3B shows the further alternative body care product according to the invention with the handle unit in a second alternative implementation, with an application unit and the connection unit, in a schematic 3D view from the front,

FIG. 3C shows the further alternative body care product according to the invention with the handle unit in a third alternative implementation, with an application unit and with the connection unit, in a schematic 3D view from the front,

FIG. 4A shows a handle unit of a further alternative body care product according to the invention that is realized as a razor, in a schematic 3D view from the front,

FIG. 4B shows the handle unit of the further alternative body care product according to the invention, in a schematic 3D view from the rear,

FIG. 4C shows the handle unit of the further alternative body care product according to the invention, in a plan view from the front,

FIG. 4D shows the handle unit of the further alternative body care product according to the invention, in a side view,

FIG. 4E shows the handle unit of the further alternative body care product according to the invention, in a plan view from the rear,

FIG. 4F shows an application unit and a connection unit of the further alternative body care product according to the invention, in a schematic 3D view from the front,

FIG. 4G shows the application unit and the connection unit of the further alternative body care product according to the invention, in a schematic 3D view from the rear,

FIG. 4H shows the application unit and the connection unit of the further alternative body care product according to the invention, in a plan view from the front,

FIG. 4I shows the application unit and the connection unit of the further alternative body care product according to the invention, in a plan view from the side,

FIG. 4J shows the application unit and the connection unit of the further alternative body care product according to the invention, in a plan view from the rear,

FIG. 4K shows the further alternative body care product according to the invention with the handle unit, the application unit and the connection unit, in a schematic 3D view from the front,

FIG. 4L shows the further alternative body care product according to the invention with the handle unit, the application unit and the connection unit, in a schematic 3D view from the rear,

FIG. 4M shows the further alternative body care product according to the invention with the handle unit, the application unit and the connection unit, in a plan view from the front,

FIG. 4N shows the further alternative body care product according to the invention with the handle unit, the application unit and the connection unit, in a side view,

FIG. 4O shows the further alternative body care product according to the invention with the handle unit, the application unit and the connection unit, in a schematic sectional illustration along the section line F-F,

FIG. 5A shows a handle unit of a further alternative body care product according to the invention that is realized as an application brush, in a schematic 3D view from the front,

FIG. 5B shows the handle unit of the further alternative body care product according to the invention, in a schematic 3D view from the rear,

FIG. 5C shows an application unit and a connection unit of the further alternative body care product according to the invention, in a schematic 3D view from the front,

FIG. 5D shows the application unit and the connection unit of the further alternative body care product according to the invention, in a schematic 3D view from the rear,

FIG. 5E shows the application unit and the connection unit of the further alternative body care product according to the invention, in a schematic sectional illustration along the section line G-G,

FIG. 5F shows the further alternative body care product according to the invention with the handle unit, the application unit and the connection unit, in a schematic 3D view from the front,

FIG. 5G shows the further alternative body care product according to the invention with the handle unit, the application unit and the connection unit, in a schematic 3D view from the rear, and

FIG. 5H shows the further alternative body care product according to the invention with the handle unit, the application unit and the connection unit, in a schematic sectional illustration along the section line H-H.

FIG. 6A shows a handle unit of a further alternative body care product according to the invention that is realized as a razor, in a schematic 3D view from the front,

FIG. 6B shows an application unit and a connection unit of the further alternative body care product according to the invention, in a schematic 3D view from the front,

FIG. 6C shows the further alternative body care product according to the invention with the handle unit, the application unit and the connection unit, in a schematic 3D view from the front,

FIG. 7A shows a blank of a further alternative body care product according to the invention in a non-folded state with a handle unit in an alternative implementation,

FIG. 7B shows a further alternative body care product according to the invention in a non-folded state with a handle unit in an alternative implementation, with an application unit, in a schematic 3D view from the front,

FIG. 7C shows a handle unit with an application unit of the body care product according to the invention that is realized as a toothbrush, in a schematic 3D view from the front,

FIG. 7D shows the handle unit with an application unit of the body care product according to the invention that is realized as a toothbrush, in a schematic 3D view from the rear,

FIG. 8A shows the handle unit and the application unit of a body care product according to the invention that is realized as a flosser, in an exploded view,

FIG. 8B shows the product from FIG. 8A in an assembled arrangement,

FIG. 9 shows a further alternative body care product according to the invention, in a schematic 3D view,

FIG. 10A shows the handle unit with the application unit of a body care product according to the invention that is realized as a flosser, in a plan view, and

FIG. 10B shows the product from FIG. 10A, in a plan view with the application region shown in a sectional view.

### DESCRIPTION OF THE EXEMPLARY EMBODIMENTS

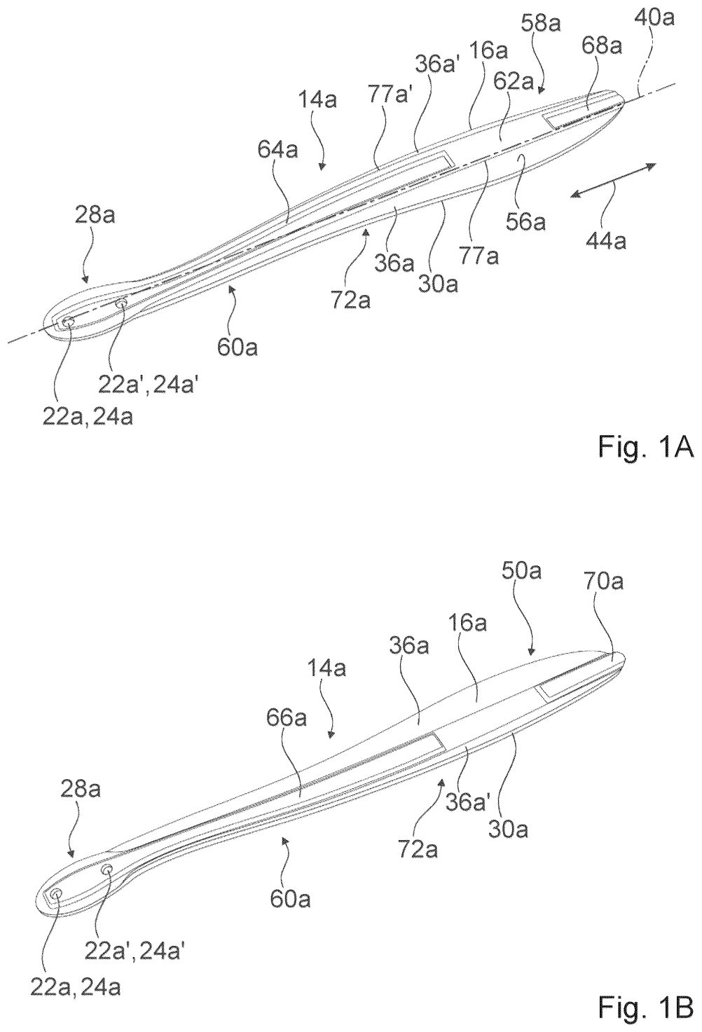

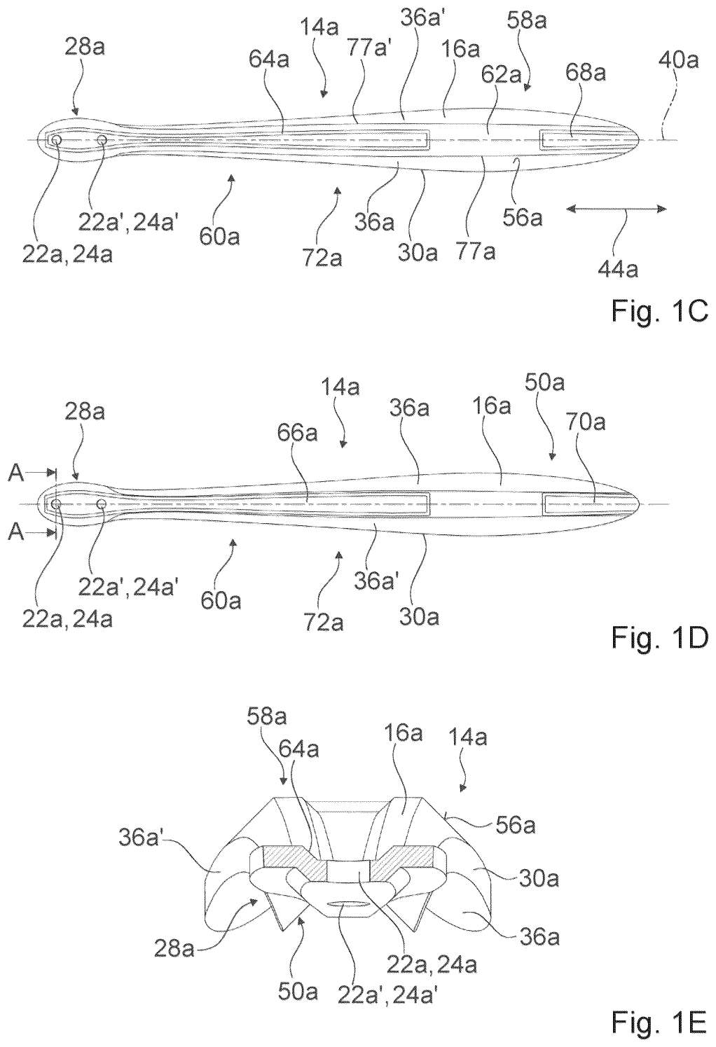

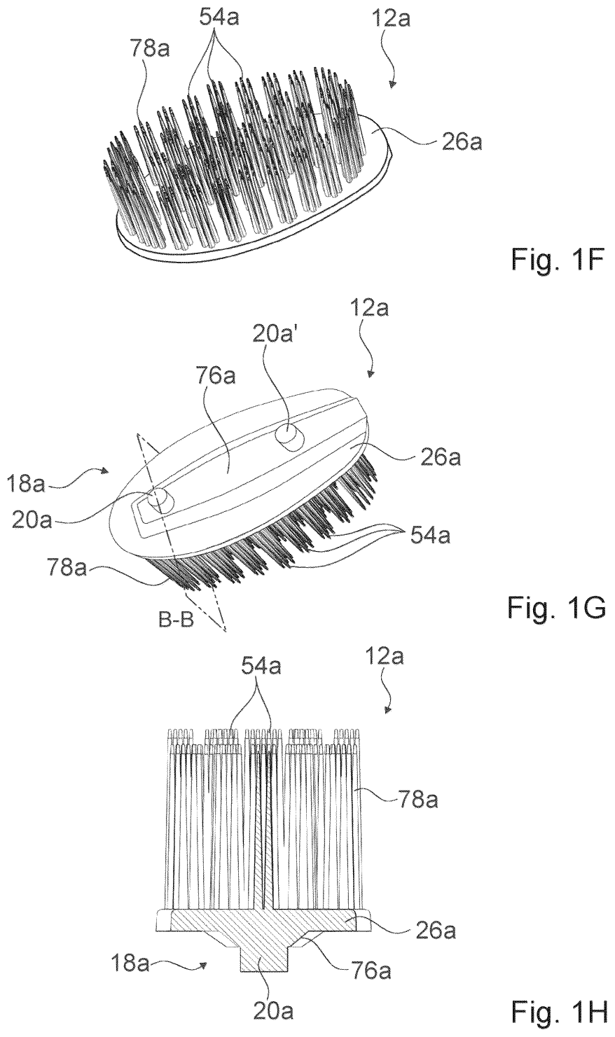

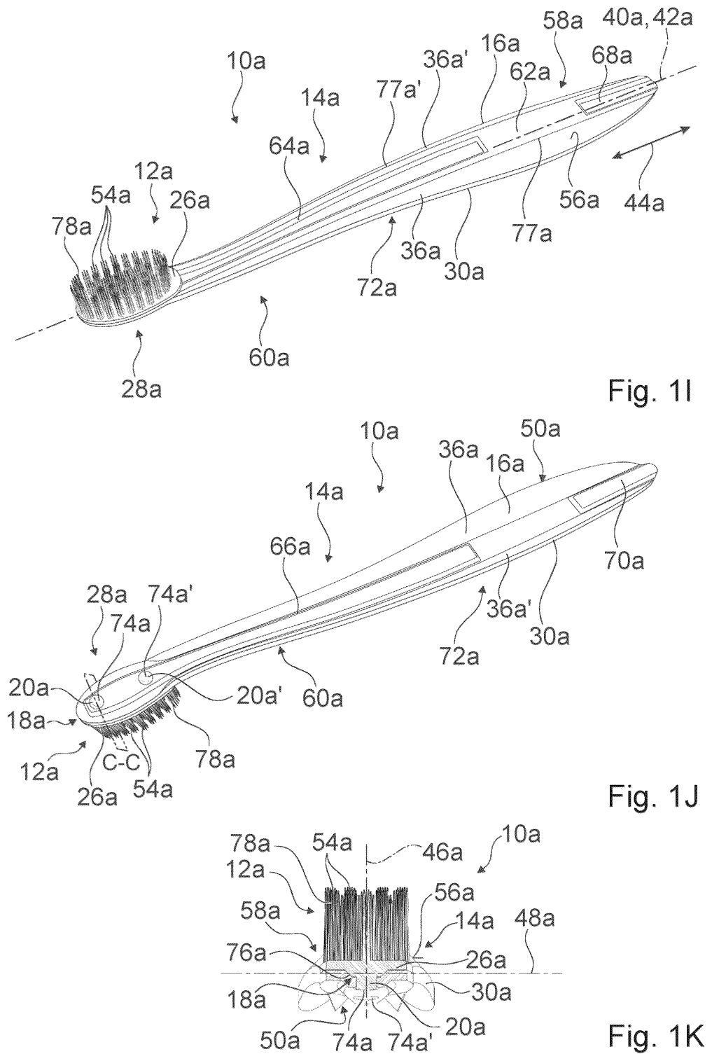

In the following, reference is made to FIGS. 1A to 1K, which show different views and manufacturing stages of a body care product 10a. Due to the different views, some elements are not shown in all figures and are accordingly not in all figures provided with reference numerals.

FIG. 1A shows a handle unit 14a of the body care product 10a in a schematic perspective illustration. The body care product 10a is realized as an oral hygiene means. The body care product 10a is in the present case realized as a toothbrush.

The body care product 10a comprises an application unit 12a. The body care product 10a furthermore comprises the handle unit 14a, which has a material handle body 16a. The body care product 10a also comprises a connection unit 18a, which connects the application unit 12a with the handle unit 14a.

The body care product 10a has a longitudinal axis 42a, a height axis 46a and a width axis 48a. The longitudinal axis 42a is arranged in parallel to a main extension direction 44a of the body care product 10a. If the body care product 10a is placed with a rear side 50a on a flat surface, such that the longitudinal axis 42a is arranged in parallel to the surface, the height axis 46a is arranged perpendicular to the longitudinal axis 42a and perpendicular to the surface and to the width axis 48a. The width axis 48a is arranged perpendicular to the longitudinal axis 42a and perpendicular to the height axis 46a. In the present case, the body care product 10a has a length, in particular parallel to the longitudinal axis 42a and measured parallel to the bearing surface, of 140 mm to 210 mm and preferably of 165 mm to 185 mm. The body care product 10a furthermore has a maximum height, in particular measured parallel to the height axis 46a and perpendicular to the bearing surface, of 12 mm to 25 mm and preferably of 15 mm to 19 mm. The body care product 10a further has a width in a point of maximum width, in particular parallel to the width axis 48a, of 10 mm to 25 mm and preferably of 13 mm to 18 mm.

The application unit 12a forms a brush head of the body care product 10a. In the present case, the application unit 12a is realized as a toothbrush head. The application unit 12a in particular has a base body 26a that is realized as a bristle carrier. The base body 26a of the application unit 12a is completely formed of a plastic component, here of a material for injection-molded bristles. However, it would also be conceivable that the base body 26a of the application unit 12a is formed of a hard component and a soft component. The base body 26a of the application unit 12a forms a bristle carrier. The application unit 12a further has several bristles 78a which are realized together with the base body 26a. The application unit 12a is produced in an injection-molding procedure and the base body 26a as well as bristle bundles 54a which consist of bristles 78a are formed. Alternatively, the bristle bundles 54a may also be accommodated in the base body 26a. Alternatively, recesses may be formed on a front side 58a of the base body 26a. The recesses are configured for accommodating the bristle bundles 54a, which are fixed in the conventional anchor-punching procedure. In that case, the base body 26a has a plurality of recesses. The recesses are realized as bristle holes. Bristles for the bristle bundles 54a may be any suitable bristles which, as described above, may for example be punched on in the anchor-punching procedure. In principle, the use of AFT would also be conceivable. In the case of AFT, a recess is provided in the base body 26a, which is configured for receiving a bristle platelet in parallel to the handle unit 14a. The platelet is in particular produced by means of an injection-molding procedure, wherein the platelet is already provided with bristles prior to the fastening. The platelet is configured to be anchored in the recess of the base body 26a, in particular by means of welding, or to be arranged by means of other connection techniques and/or arranged outside a recess. The bristle bundles 54a may differ with regard to their length, their composition, their number of bristles, a bristle material, a color, a surface structuring and the like.

The application unit 12a is arranged at the top side of the body care product 10a. The application unit 12a forms an uppermost point of the body care product 10a. The application unit 12a is completely made of a plastic component, in the present case of a material for injection-molded bristles. However, it would also be conceivable that the application unit 12a is made partially of a soft component and partially of a hard component. With regard to suitable soft and/or hard components, reference is made to the above description.

The handle unit 14a is configured to be gripped by the operator. The handle unit 14a comprises the material handle body 16a. The material handle body 16a of the handle unit 14a has a head region 28a. Furthermore, the material handle body 16a of the handle unit 14a has a handle region 72a and a neck region 60a that is arranged between the handle region 72a and the head region 28a. The head region 28a is configured for a connection of the application unit 12a. The handle unit 14a comprises at least one handle surface 56a, which forms a surface of the material handle body 16a. The handle surface 56a extends on the front side 58a from the neck region 60a of the handle body 16a to the lower end of the body care product 10a at the bottom side. The handle surface 56a extends over the handle region 72a of the material handle body 16a. The handle surface 56a forms a cambered and/or convex shape on the front side 58a along the longitudinal axis 42a. The cambered shape is formed in an inverted manner on the rear side 50a along the longitudinal axis 42a. On the rear side 50a the handle body 16a is concavely shaped in a region of the handle surface 56a. In a region of the handle surface 56a, the handle body 16a has an oval basic shape. The handle body 16a is three-dimensionally deformed in order to form the cambered convex shape. The material handle body 16a of the handle unit 14a further comprises two stabilizing walls 36a, 36a′, which are angled with respect to a main extension plane of the handle unit 14a and extend substantially parallel to a longitudinal axis 40a of the handle unit 14a. The stabilizing walls 36a, 36a′ are in each case angled over a folding edge 77a, 77a′ with respect to a remaining portion of the material handle body 16a. A central region of the material handle body 16a is arranged between the stabilizing walls 36a, 36a′. The stabilizing walls 36a, 36a′ extend in each case over the handle region 72a and the neck region 60a of the material handle body 16a. The stabilizing walls 36a, 36a′ are arranged on an outer edge of the material handle body 16a that extends substantially parallel to the longitudinal axis 40a. The stabilizing walls 36a, 36a′ are angled, perpendicular to the longitudinal axis 40a of the handle unit 14a, at an angle of at least 10°, preferably at least 20°, with respect to a main extension plane of the handle body 16a. The stabilizing walls 36a, 36a′ in each case form a free outer edge of the material handle body 16a.

In particular, a lettering and/or a logo could be arranged in a central or rear region of the handle surface 56a. The handle body 16a forms a plateau 62a on the handle surface 56a. The plateau 62a serves for receiving a lettering and/or a logo. The plateau 62a is arranged at least partially centrally on the handle surface 56a. The plateau 62a is arranged in a region of a maximum width of the body care product 10a.

From a bottom side to a top side, a maximum width of the body care product 10a is arranged in the first 30% of the main extension of the body care product 10a. After the maximum width, the body of the body care product 10a decreases continuously in width rearwards as far as the rear end. After the maximum width, the body of the body care product 10a decreases continuously in width frontwards to the application unit 12a, the handle unit 14a and the neck region 60a as far as the head base of the head region 28a. The handle unit 14a has in its height, in particular parallel to the height axis 46a, an approximately uniform material thickness.

The handle body 16a furthermore has a substantially constant material thickness. Furthermore, the handle body 16a comprises a first groove 64a which extends on the front side 58a from the application unit 12a over the neck region 60a and over the handle surface 56a as far as the plateau 62a. The first groove 64a extends centrally in parallel to the longitudinal axis 40a of the handle unit 14a. Along an extension parallel to the longitudinal axis 40a, the first groove 64a has an at least approximately constant cross section, wherein the first groove 64a partly tapers in the neck region 60a. The first groove 64a has a trapezoidal cross section which widens towards the front side 58a. A first web-shaped elevation 66a corresponding to the first groove 64a is formed on the rear side 50a of the material handle body 16a. The first web-shaped elevation 66a is here in particular due to the constant material thickness during production of the first groove 64a.

The handle body 16a moreover comprises a second groove 68a, which extends on the front side 58a from a lower end of the handle unit 14a that faces away from the application unit 12a over the handle surface 56a as far as the plateau 62a. The second groove 68a extends centrally in parallel to the longitudinal axis 40a of the handle unit 14a. Along an extension parallel to the longitudinal axis 40a, the second groove 68a has an at least approximately constant cross section. The second groove 68a has a trapezoidal cross section which widens towards the front side 58a. The second groove 68a extends substantially in alignment with the first groove 64a, wherein the first groove 64a and the second groove 68a are interrupted by the plateau 62a. A second web-shaped elevation 70a corresponding to the second groove 68a is formed on the rear side 50a of the material handle body 16a. The second web-shaped elevation 70a is here in particular formed during production of the second groove 68a due to the constant material thickness.

The material handle body 16a is at least largely made of a paper material. The material handle body 16a consists completely of a paper material. The material handle body 16a of the handle unit 14a comprises at least one layer 30a of a paper material, which is formed three-dimensionally. The material handle body 16a consists of precisely one layer 30a of a paper material, but a multilayer structure would also be conceivable.

The material handle body 16a of the handle unit 14a is formed in substantially S-shaped curved fashion along a longitudinal direction of the handle unit 14a. The material handle body 16a has an S-shaped longitudinal form in a longitudinal section and/or in a longitudinal view. The profile of the handle unit 14a is optimized for an application and for user ergonomics. The material handle body 16a has a stepless continuous rounded shape in the longitudinal direction.

The body care product 10a furthermore comprises the connection unit 18a. The connection unit 18a is configured for a form-fitting connection to the material handle body 16a of the handle unit 14a. In addition to the form-fitting connection, a force-fitting and/or material-bonding connection may also be provided. It is in particular conceivable that the connection unit 18a is additionally at least partly glued, welded or cast with the material handle body 16a of the handle unit 14a. It would also be conceivable that a connection is created by injection-molding and/or overmolding or by clamping.

The connection unit 18a comprises at least one form-fitting element 20a, 20a′. By way of example, the connection unit 18a comprises two form-fitting elements 20a, 20a′. The form-fitting elements 20a, 20a′ are configured to respectively engage in a recess 22a, 22a′ of the application unit 12a and/or of the handle unit 14a. The form-fitting elements 20a, 20a′ are configured to respectively engage in a recess 22a, 22a′ of the handle unit 14a. The recesses 22a, 22a′ of the handle unit 14a are realized as pass-through recesses 24a, 24a′. The material handle body 16a delimits the two pass-through recesses 24a, 24a′. The pass-through recesses 24a, 24a′ are introduced in the head region 28a of the material handle body 16a. The pass-through recesses 24a, 24a′ are arranged one behind the other along the longitudinal axis 40a of the handle unit 14a. The pass-through recesses 24a, 24a′ are arranged in the first groove 64a. The pass-through recesses 24a, 24a′ are configured to receive the form-fitting elements 20a, 20a′ of the connection unit 18a. The pass-through recesses 24a, 24a′ are in each case realized as a circular-cylindrical recess 22a, 22a′. The form-fitting elements 20a, 20a′ likewise have a cylindrical basic shape. The form-fitting elements 20a, 20a′ are in each case realized as a rivet. The form-fitting elements 20a, 20a′ are in each case realized as a plastic rivet. In a state when they are connected to the handle unit 14a, the form-fitting elements 20a, 20a′ in each case have a shaft and a form-fitting head 74a, 74a′. The form-fitting head 74a, 74a′ of the form-fitting elements 20a, 20a′ has a larger diameter than the pass-through recesses 24a, 24a′ and is arranged on a side of the handle unit 14a that faces away from the application unit 12a. The form-fitting heads 74a, 74a′ of the form-fitting elements 20a, 20a′ prevent a retraction of the connection unit 18a and thus a removal of the connection unit 18a from the handle unit 14a.

During production, the handle unit 14a and the connection unit 18a are provided. Then the handle unit 14a and the connection unit 18a are brought together, wherein the form-fitting elements 20a, 20a′ extend through the pass-through recesses 24a, 24a′. Following this, the penetrating shafts of the form-fitting elements 20a, 20a′ are deformed. Herein various deformation options, deemed expedient by someone skilled in the art, are conceivable, such as for example by pressure, with the form-fitting elements 20a, 20a′ being pressed and/or slightly crushed; or by pressure and heat, with the form-fitting elements 20a, 20a′ being deformed and/or slightly crushed by means of a heated punch. Herein the pressure is in particular selected in such a way that a controlled crushing takes place. When the pressure is applied, in particular an expansion of the compressed form-fitting elements 20a, 20a′ is controlled. Preferably a punch for this purpose is in particular not flat but concavely curved. A round shape of the contact surface in particular controls the flowing of the material during the deformation of the form-fitting elements 20a, 20a′.

The material handle body 16a of the handle unit 14a furthermore has in the head region 28a a depression configured for a positioning of the connection unit 18a relative to the handle unit 14a. The depression is formed by a partial region of the first groove 64a. The depression is realized as an elongate depression in the head region 28a, which extends parallel to the longitudinal axis 40a of the handle unit 14a. The connection unit 18a comprises a web 76a which corresponds to the depression, respectively the first groove 64a, and extends parallel to the longitudinal axis 42a of the body care product 10a over a large portion of an extension of the connection unit 18a. The web 76a has a trapezoidal cross section. Furthermore, the web 76a is widened in a central region. A shape of the web 76a is adapted to a shape of the first groove 64a in the head region 28a. The form-fitting elements 20a, 20a′ are arranged on the web 76a. The depression and the web 76a are configured for a pre-positioning of the connection unit 18a on the handle unit 14a and moreover serve for a partial absorption of transverse forces. The connection of the depression to the web 76a realizes a form-fitting connection of the connection unit 18a. The form-fitting elements 20a, 20a′ realize a further form-fitting connection of the connection unit 18a with the handle unit 14a.

The application unit 12a comprises the base body 26a. The base body 26a is fixedly connected with the connection unit 18a. The base body 26a is integrally connected with the connection unit 18a. The base body 26a is formed in a one-part implementation with the connection unit 18a. The connection unit 18a is integrated in the base body 26a. The form-fitting elements 20a, 20a′ are realized integrally with the base body 26a of the application unit 12a. The form-fitting elements 20a, 20a′ are realized in a one-part implementation with the base body 26a of the application unit 12a. The web 76a is likewise realized in a one-part implementation with the base body 26a of the application unit 12a. The bristle bundles 54a are also formed on the base body 26a, respectively together with the base body 26a.

The structure of the body care product 10a is provided with a number of lays and a number of layers, as mentioned in the description.

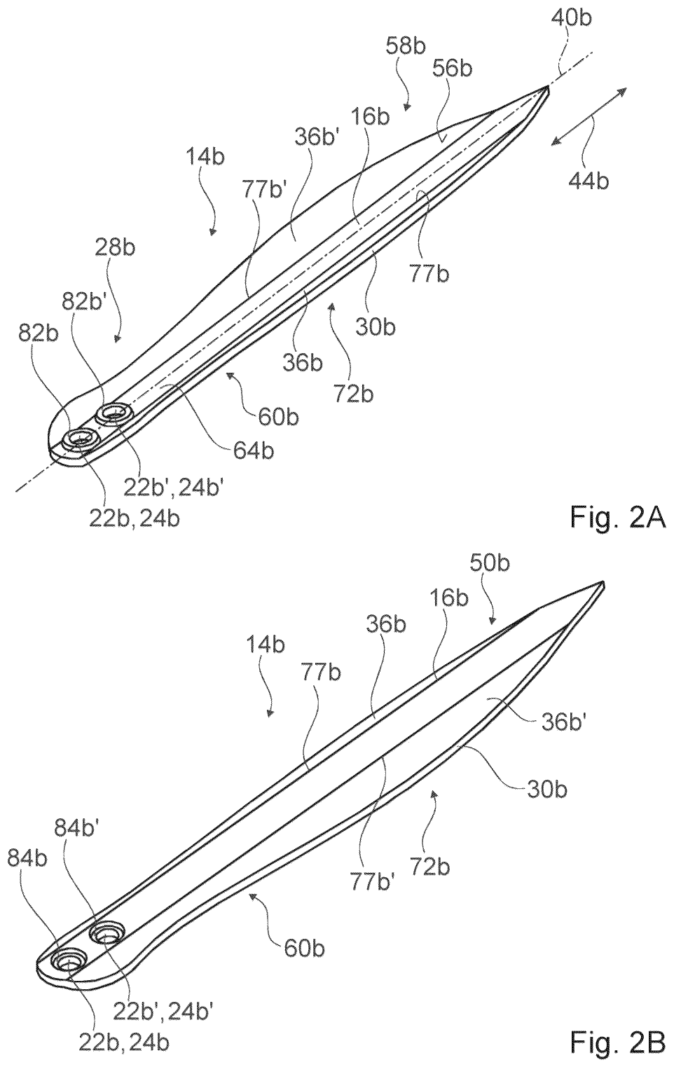

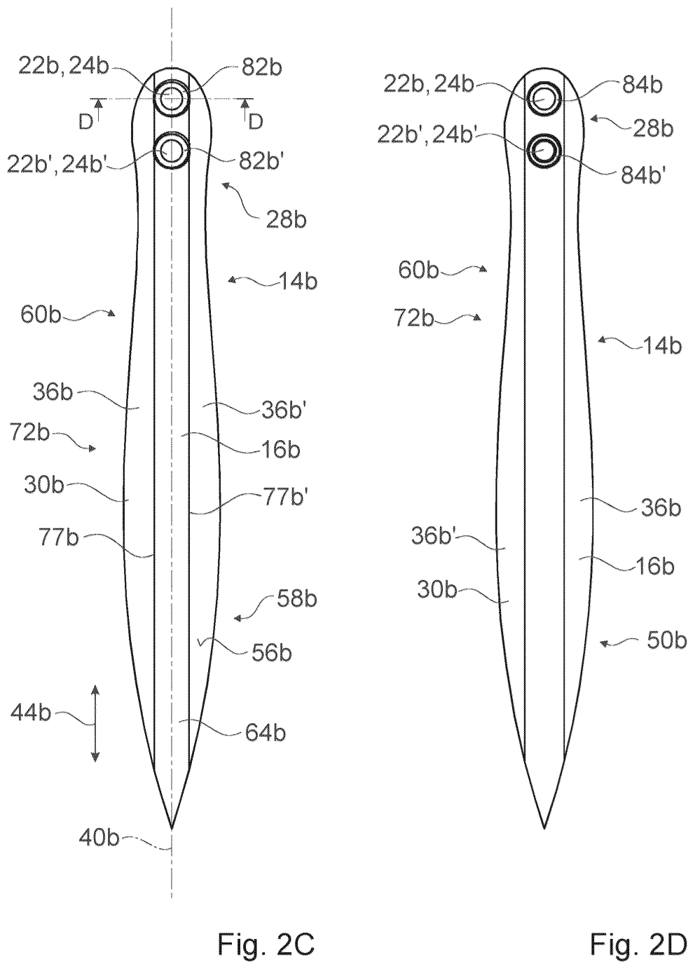

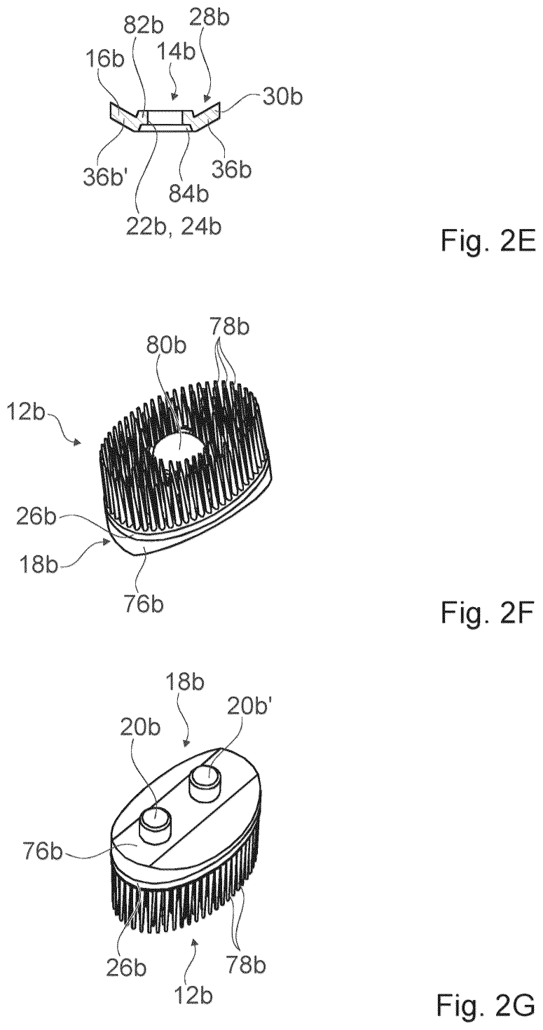

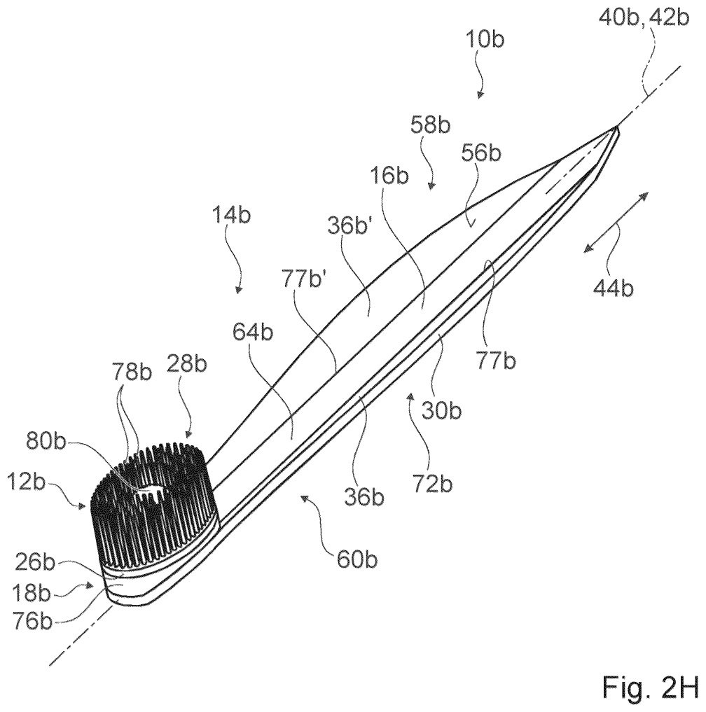

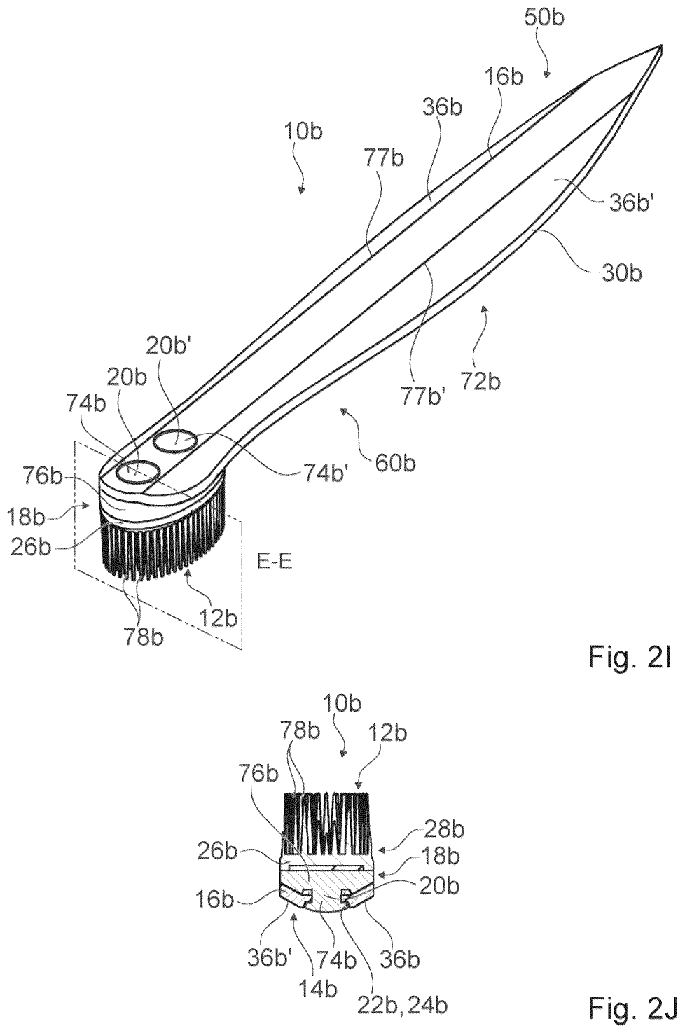

In FIGS. 2A to 6C four further exemplary embodiments of the invention are shown. The following descriptions are substantially limited to the differences between the exemplary embodiments, wherein with regard to components, features and functions that remain the same reference may be made to the description of the other exemplary embodiments, in particular of FIGS. 1A to 1K. In order to distinguish between the exemplary embodiments, the letter a in the reference numerals of the exemplary embodiment of FIGS. 1A to 1K has been replaced by the letters b to e in the reference numerals of the exemplary embodiments of FIGS. 2A to 6C. With regard to components having the same denomination, in particular with regard to components having the same reference numerals, reference may in principle also be made to the drawings and/or the description of the other exemplary embodiments, in particular of FIGS. 1A to 1K.

In the following, reference will be made to FIGS. 2A to 2J, which show different views and manufacturing stages of the body care product 10b. Due to the different views, some elements are not shown in all the figures and are accordingly not in all the figures provided with reference numerals.

FIG. 2A shows a handle unit 14b of a body care product 10b in a schematic perspective illustration. The body care product 10b is realized as an oral hygiene means. The body care product 10b is in the present case realized as a toothbrush. The body care product 10b is realized as a disposable toothbrush. The body care product 10b is realized as a traveling and/or compact toothbrush. The body care product 10b has formed, on its side opposite the application unit 12b, a tip which quasi forms a further application unit. The tip serves as a toothpick tip, and thus that the body care product 10b has two oral hygiene functions.

The body care product 10b comprises an application unit 12b. The body care product 10b further comprises a handle unit 14b which has a material handle body 16b. The body care product 10b also comprises a connection unit 18b, which connects the application unit 12b with the handle unit 14b.

The body care product 10b has a longitudinal axis 42b, a height axis and a width axis. The longitudinal axis 42b is arranged in parallel to a main extension direction 44b of the body care product 10b. If the body care product 10b is placed with a rear side 50b on a flat surface, such that the longitudinal axis 42b is arranged parallel to the surface, the height axis is arranged perpendicular to the longitudinal axis 42b and perpendicular to the surface and to the width axis. The width axis is arranged perpendicular to the longitudinal axis 42b and perpendicular to the height axis. In the present case, the body care product 10b has a length, in particular parallel to the longitudinal axis 42b and measured parallel to the bearing surface, of 60 mm to 100 mm, preferably of 75 mm to 90 mm. Furthermore, the body care product 10b has a maximum height, in particular measured parallel to the height axis and perpendicular to the bearing surface, of 6 mm to 15 mm and preferably of 8 mm to 12 mm. The body care product 10b further has in a point of maximum width a width, in particular parallel to the width axis, of 7 mm to 15 mm and preferably of 9 mm to 13 mm.

The application unit 12b forms a brush head of the body care product 10b. In the present case, the application unit 12b is realized as a toothbrush head. The application unit 12b in particular has a base body 26b that is realized as a bristle carrier. The base body 26b forms a connection to the connection unit 18b. The base body 26b of the application unit 12b forms a bristle carrier. The application unit 12b further comprises several bristles 78b, which are arranged on the base body 26b. The bristles 78b are injection-molded to the base body 26b. The bristles 78b consist of material for injection-molded bristles; this material also forms the base body 26b. The application unit 12b further comprises a functional element 80b that is arranged on the base body 26b between the bristles 78b. The functional element 80b is realized, for example, as a capsule for receiving mouthwash and/or toothpaste, which is configured to burst open during use and to release a content.

The handle unit 14b is configured to be gripped by the operator. The handle unit 14b comprises the material handle body 16b. The material handle body 16b of the handle unit 14b has a head region 28b. Furthermore, the material handle body 16b of the handle unit 14b has a handle region 72b and a neck region 60b that is arranged between the handle region 72b and the head region 28b. The head region 28b is configured for a connection of the application unit 12b. The handle unit 14b comprises at least one handle surface 56b, which forms a surface of the material handle body 16b. The handle surface 56b forms a concave shape on a front side 58b along the longitudinal axis 42b. On a rear side 50b the handle body 16b is convexly shaped in a region of the handle surface 56b. In a region of the handle surface 56b, the handle body 16b has an oval basic shape. The handle body 16b is realized in three-dimensional fashion. The material handle body 16b of the handle unit 14b further comprises two stabilizing walls 36b, 36b′, which are angled with respect to a main extension plane of the handle unit 14b and extend substantially parallel to a longitudinal axis 40b of the handle unit 14b. The stabilizing walls 36b, 36b′ are in each case angled toward the front side 58b over a folding edge 77b, 77b′ with respect to a remaining portion of the material handle body 16b, such that a depression extending over the entire handle body 16b is created on the front side 58b. A central region of the material handle body 16b is arranged between the stabilizing walls 36b, 36b′. The stabilizing walls 36b, 36b′ in each case extend over the handle region 72b, the neck region 60b and the head region 28b of the material handle body 16b. The stabilizing walls 36b, 36b′ are arranged on an outer edge of the material handle body 16b that extends substantially parallel to the longitudinal axis 40b. The stabilizing walls 36b, 36b′ are angled, perpendicular to the longitudinal axis 40b of the handle unit 14b, at an angle of at least 10°, preferably at least 20°, with respect to a main extension plane of the handle body 16b. The stabilizing walls 36b, 36b′ in each case form a free outer edge of the material handle body 16b.

The material handle body 16b is at least largely made of a paper material. The material handle body 16b consists completely of a paper material. The material handle body 16b of the handle unit 14b has at least one layer 30b of a paper material, which is realized in three-dimensional fashion. The material handle body 16b consists of precisely one layer 30b of a paper material.

The body care product 10b furthermore comprises the connection unit 18b. The connection unit 18b is configured for a form-fitting connection with the material handle body 16b of the handle unit 14b.

The connection unit 18b comprises at least one form-fitting element 20b, 20b′. By way of example, the connection unit 18b comprises two form-fitting elements 20b, 20b′. The form-fitting elements 20b, 20b′ are configured to respectively engage in a recess 22b, 22b′ of the application unit 12b and/or of the handle unit 14b. The form-fitting elements 20b, 20b′ are configured to respectively engage in a recess 22b, 22b′ of the handle unit 14b. The recesses 22b, 22b′ of the handle unit 14b are realized as pass-through recesses 24b, 24b′. The material handle body 16b delimits the two pass-through recesses 24b, 24b′. The pass-through recesses 24b, 24b′ are introduced in the head region 28b of the material handle body 16b. The pass-through recesses 24b, 24b′ are arranged one behind the other along the longitudinal axis 40b of the handle unit 14b. The pass-through recesses 24b, 24b′ are in each case introduced in a cylindrical elevation of the material handle body 16b, which on the front side 58b respectively forms an annular elevation 82b, 82b′ around the pass-through recesses 24b, 24b′ and on the rear side 50b respectively forms an annular depression 84b, 84b′ around the pass-through recesses 24b, 24b′. The depressions 84b, 84b′ respectively form a ledge in the pass-through recesses 24b, 24b′. The pass-through recesses 24b, 24b′ are configured to receive the form-fitting elements 20b, 20b′ of the connection unit 18b. The pass-through recesses 24b, 24b′ are in each case realized as a circular-cylindrical recess 22b, 22b′. The form-fitting elements 20b, 20b′ likewise have a cylindrical basic shape. The form-fitting elements 20b, 20b′ are in each case realized as a rivet. The form-fitting elements 20b, 20b′ are in each case realized as a plastic rivet. In a state when they are connected to the handle unit 14b, the form-fitting elements 20b, 20b′ in each case have a shaft and a deformed form-fitting head 74b, 74b′. The form-fitting head 74b, 74b′ of the form-fitting elements 20b, 20b′ has a larger diameter than the pass-through recesses 24b, 24b′ and is arranged on a side of the handle unit 14b that faces away from the application unit 12b. The form-fitting heads 74b, 74b′ of the form-fitting elements 20b, 20b′ prevent a retraction of the connection unit 18b. The form-fitting heads 74b, 74b′ of the form-fitting elements 20b, 20b′ respectively extend into the depressions 84b, 84b′ around the pass-through recesses 24b, 24b′ and thus preferably do not protrude beyond the further handle surface 56b.

The material handle body 16b of the handle unit 14b furthermore has in the head region 28b the depression configured for a positioning of the connection unit 18b relative to the handle unit 14b. The depression is realized as an elongate depression in the head region, which extends parallel to the longitudinal axis 40b of the handle unit 14b. The connection unit 18b comprises a web 76b which corresponds to the depression, respectively to the first groove 64b, and extends parallel to a longitudinal axis 42b of the body care product 10b over a large portion of an extension of the connection unit 18b. The web 76b has a trapezoidal cross section. A shape of the web 76b is adapted to a shape of the first groove 64b in the head region 28b. The form-fitting elements 20b, 20b′ are arranged on the web 76b. The depression and the web 76b are configured for a pre-positioning of the connection unit 18b on the handle unit 14b and moreover serve for a partial absorption of transverse forces. The connection of the depression to the web 76b creates a form-fitting connection of the connection unit 18b. Furthermore, the annular elevations 82b, 82b′ can also serve for a form-fitting connection. For this purpose, annular depressions (not visible in detail), into which the annular elevations 82b, 82b′ engage, may be respectively arranged in the web 76b around the form-fitting elements 20b, 20b′. The form-fitting elements 20b, 20b′ form a further form-fitting connection of the connection unit 18b with the handle unit 14b.

The application unit 12b comprises the base body 26b. The base body 26b is fixedly connected with the connection unit 18b. The base body 26b is integrally connected with the connection unit 18b. The base body 26b is connected with the connection unit 18b, in particular with the web 76b, by means of an adhesive connection and/or welded connection.

The structure of the body care product 10b is provided with a number of lays and a number of layers, as mentioned in the description.

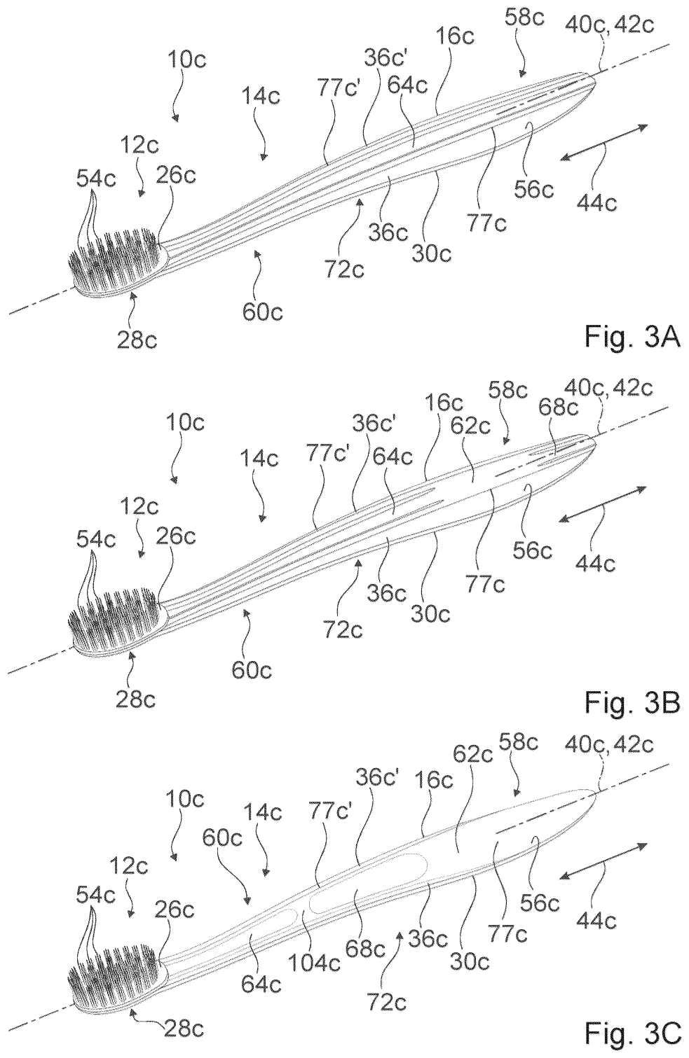

FIGS. 3A, 3B and 3C respectively show a further alternative body care product 10c according to the invention. The body care product 10c is realized as an oral hygiene means. The body care product 10c is in the present case realized as a toothbrush.

The body care product 10c comprises an application unit 12c. The body care product 10c furthermore comprises a handle unit 14c, which has a material handle body 16c. The body care product 10c also comprises a connection unit 18c, which connects the application unit 12c to the handle unit 14c.

The handle unit 14c is configured to be gripped by the operator. The handle unit 14c comprises the material handle body 16c. The material handle body 16c of the handle unit 14c comprises a head region 28c. The material handle body 16c of the handle unit 14c further comprises a handle region 72c and a neck region 60c that is arranged between the handle region 72c and the head region 28c. The head region 28c is configured for a connection of the application unit 12c.

The material handle body 16c is at least largely made of a paper material. The material handle body 16c consists completely of a paper material. The material handle body 16c of the handle unit 14c comprises at least one layer 30c of a paper material, which is formed three-dimensionally. The material handle body 16c consists of precisely one layer 30c of a paper material; however, a multilayer structure would also be conceivable.

The material handle body 16c of the handle unit 14c is formed in substantially S-shaped curved fashion along a longitudinal direction of the handle unit 14c. The material handle body 16c has an S-shaped longitudinal form in a longitudinal section and/or in a longitudinal view. The profile of the handle unit 14c is optimized for an application and for user ergonomics. The material handle body 16c has in the longitudinal direction a stepless continuous rounded shape.

FIGS. 3A, 3B and 3C respectively show modifications of the handle unit 14a of the first exemplary embodiment of FIGS. 1A to 1K. In the following therefore in particular only the differences between the various implementations of the handle unit 14c will be described, in particular with respect to the handle unit 14a of the first exemplary embodiment of FIGS. 1A to 1K.

The handle unit 14c of FIG. 3A comprises at least one handle surface 56c, which forms a surface of the material handle body 16c. The handle surface 56c forms a cambered and/or convex shape on a front side 58c along a longitudinal axis 42c. The cambered shape is formed in an inverted fashion on the rear side along the longitudinal axis 42c. On the rear side the handle body 16c is concavely shaped in a region of the handle surface 56c. In a region of the handle surface 56c, the handle body 16c has an oval basic shape. In order to create the cambered convex shape, the handle body 16c is three-dimensionally deformed. The material handle body 16c of the handle unit 14c further comprises two stabilizing walls 36c, 36c′, which are angled with respect to a main extension plane of the handle unit 14c and extend substantially parallel to a longitudinal axis 40c of the handle unit 14c. The stabilizing walls 36c, 36c′ are in each case angled over a folding edge 77c, 77c′ with respect to a remaining portion of the material handle body 16c. A middle region of the material handle body 16c is arranged between the stabilizing walls 36c, 36c′. The stabilizing walls 36c, 36c′ in each case extend over the handle region 72c and the neck region 60c of the material handle body 16c. The handle body 16c further has a substantially constant material thickness. The handle body 16c moreover comprises a groove 64c, which extends on the front side 58c from the application unit 12c over the neck region 60c and over the handle surface 56c as far as a lower end of the handle unit 14c. The groove 64c runs centrally in parallel to the longitudinal axis 40c of the handle unit 14c. Along its extension parallel to the longitudinal axis 40c, the groove 64c has an at least approximately constant cross section, wherein the groove 64c partly tapers in the neck region 60c. The groove 64c has a trapezoidal cross section, which widens towards a front side 58c. On a rear side of the material handle body 16c, a web-shaped elevation is formed that corresponds to the groove 64c. The web-shaped elevation is here in particular formed during production of the groove 64c due to the constant material thickness.

The handle unit 14c of FIG. 3B comprises at least one handle surface 56c, which forms a surface of the material handle body 16c. The handle surface 56c forms a cambered and/or convex shape on the front side 58c along the longitudinal axis 42c. The cambered shape is formed in an inverted fashion on the rear side along the longitudinal axis 42c. On the rear side the handle body 16c is concavely shaped in a region of the handle surface 56c. In a region of the handle surface 56c, the handle body 16c has an oval basic shape. The handle body 16c is three-dimensionally deformed in order to create the cambered convex shape. The material handle body 16c of the handle unit 14c further comprises two stabilizing walls 36c, 36c′, which are angled with respect to a main extension plane of the handle unit 14c and extend substantially parallel to a longitudinal axis 40c of the handle unit 14c. The stabilizing walls 36c, 36c′ are in each case angled over a folding edge 77c, 77c′ with respect to a remaining portion of the material handle body 16c. A middle region of the material handle body 16c is arranged between the stabilizing walls 36c, 36c′. The stabilizing walls 36c, 36c′ in each case extend over the handle region 72c and the neck region 60c of the material handle body 16c. The handle body 16c forms a plateau 62c on the handle surface 56c. The plateau 62c serves for receiving a lettering and/or a logo. The plateau 62c is arranged at least partially centrally on the handle surface 56c. The plateau 62c is arranged in a region of a maximum width of the body care product 10c. The handle body 16c further has a substantially constant material thickness. The handle body 16c moreover comprises a first groove 64c, which extends on the front side 58c from the application unit 12c over the neck region 60c and over the handle surface 56c as far as the plateau 62c, wherein the first groove 64c ends smoothly in the plateau 62c. The first groove 64c extends centrally in parallel to the longitudinal axis 40c of the handle unit 14c. Along an extension parallel to the longitudinal axis 40c, the first groove 64c has an at least approximately constant cross section, wherein the first groove partly tapers in the neck region 60c. The first groove 64c has a trapezoidal cross section, which widens towards a front side 58c. On a rear side of the material handle body 16c, a first web-shaped elevation is formed that corresponds to the first groove 64c. The first web-shaped elevation is here in particular formed during production of the first groove 64c due to the constant material thickness. Furthermore the handle body 16c comprises a second groove 68c, which extends on the front side 58c from a lower end of the handle unit 14c, which faces away from the application unit 12c, over the handle surface 56c as far as the plateau 62c, wherein the second groove 68c ends smoothly in the plateau 62c. The second groove 68c extends centrally in parallel to the longitudinal axis 40c of the handle unit 14c. Along an extension parallel to the longitudinal axis 40c, the second groove 68c has an at least approximately constant cross section. The second groove 68c has a trapezoidal cross section, which widens towards a front side 58c. The second groove 68c extends substantially in alignment with the first groove 64c, wherein the first groove 64c and the second groove 68c are interrupted by the plateau 62c. On a rear side of the material handle body 16c, a second web-shaped elevation is formed that corresponds to the second groove 68c. The second web-shaped elevation is here in particular formed during production of the second groove 68c due to the constant material thickness.

The handle unit 14c of FIG. 3C comprises at least one handle surface 56c, which forms a surface of the material handle body 16c. The handle surface 56c forms a cambered and/or convex shape on the front side 58c along the longitudinal axis 42c. The cambered shape is formed in an inverted fashion on the rear side along the longitudinal axis 42c. On the rear side the handle body 16c is concavely shaped in a region of the handle surface 56c. In a region of the handle surface 56c, the handle body 16c has an oval basic shape. In order to form the cambered convex shape, the handle body 16c is three-dimensionally deformed. The material handle body 16c of the handle unit 14c further comprises two stabilizing walls 36c, 36c′, which are angled with respect to a main extension plane of the handle unit 14c and extend substantially parallel to a longitudinal axis 40c of the handle unit 14c. The material handle body 16c merges smoothly, in particular free of folding edges, from a middle region into the stabilizing walls 36c, 36c′, wherein the stabilizing walls 36c, 36c′ are angled with respect to a remaining portion of the material handle body 16c. The middle region of the material handle body 16c is arranged between the stabilizing walls 36c, 36c′. The stabilizing walls 36c, 36c′ in each case extend over the handle region 72c and the neck region 60c of the material handle body 16c. The handle body 16c forms a plateau 62c on the handle surface 56c. The plateau 62c serves as a contact surface for a palm of a hand. The handle body 16c further has a thumb grip 104c. The thumb grip 104c is arranged in a transition zone from the handle region 72c to the neck region 60c. The handle body 16c further has a substantially constant material thickness. The handle body 16c moreover comprises a first groove 64c, which extends on the front side 58c from the application unit 12c over the neck region 60c as far as the thumb grip 104c, wherein the first groove 64c ends smoothly in the thumb grip 104c and in a peripheral edge of the first groove 64c. The first groove 64c runs centrally in parallel to the longitudinal axis 40c of the handle unit 14c. Along its extension parallel to the longitudinal axis 40c, the first groove 64c has an at least approximately constant cross section, wherein the first groove partly tapers in the neck region 60c. On a rear side of the material handle body 16c, a first web-shaped elevation is formed that corresponds to the first groove 64c. The first web-shaped elevation is here in particular formed during production of the first groove 64c due to the constant material thickness. The handle body 16c further comprises a second groove 68c, which extends on the front side 58c from a middle of the handle region 72c as far as the thumb grip 104c, wherein the second groove 68c ends smoothly in the thumb grip 104c and in a peripheral edge of the second groove 68c. The second groove 68c runs centrally in parallel to the longitudinal axis 40c of the handle unit 14c. Along its extension parallel to the longitudinal axis 40c, the second groove 68c has an at least approximately constant cross section. The second groove 68c extends substantially in alignment with the first groove 64c, wherein the first groove 64c and the second groove 68c are interrupted by the thumb grip 104c. On a rear side of the material handle body 16c, a second web-shaped elevation is formed that corresponds to the second groove 68c. The second web-shaped elevation is here in particular formed during production of the second groove 68c due to the constant material thickness.

The structure of the body care product 10c is provided with a number of lays and a number of layers, as mentioned in the description.

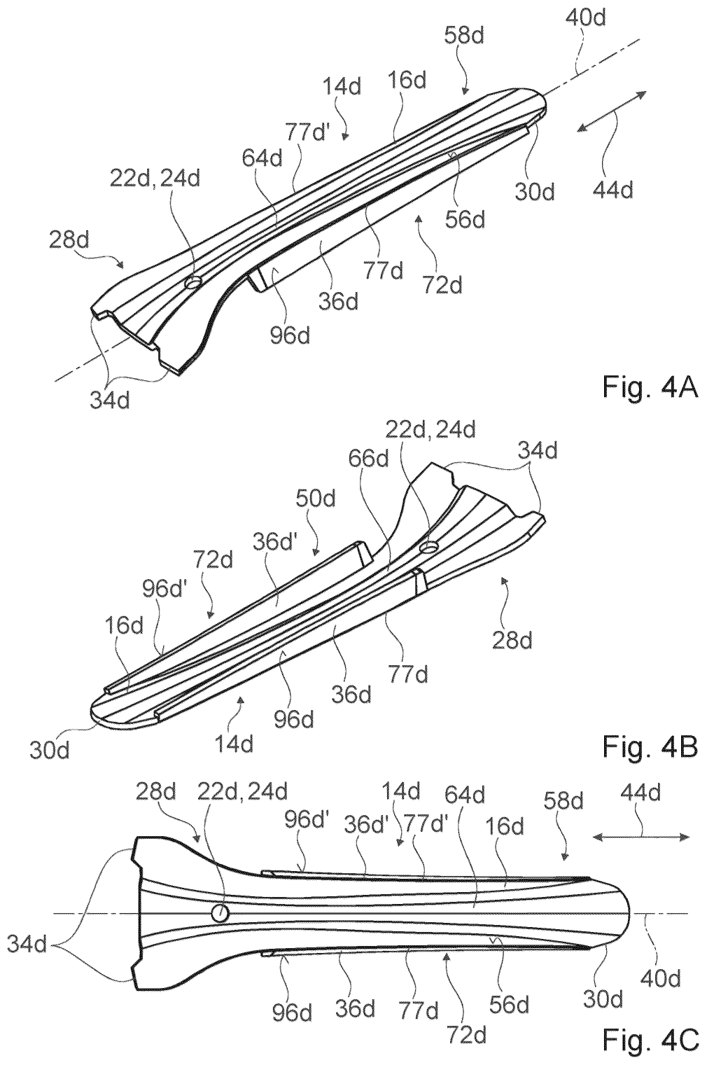

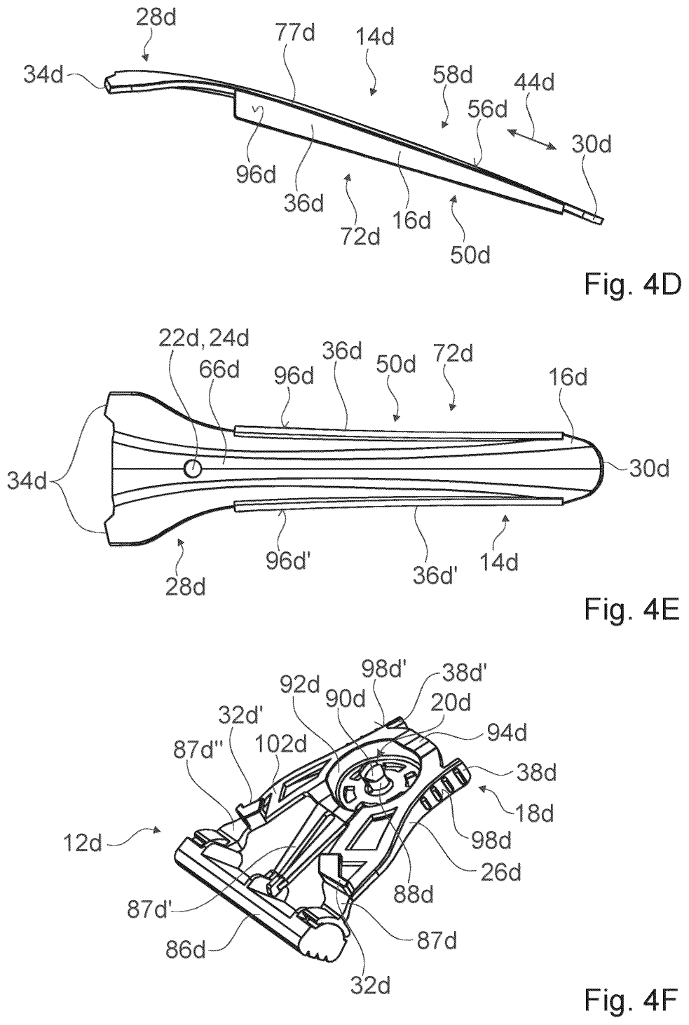

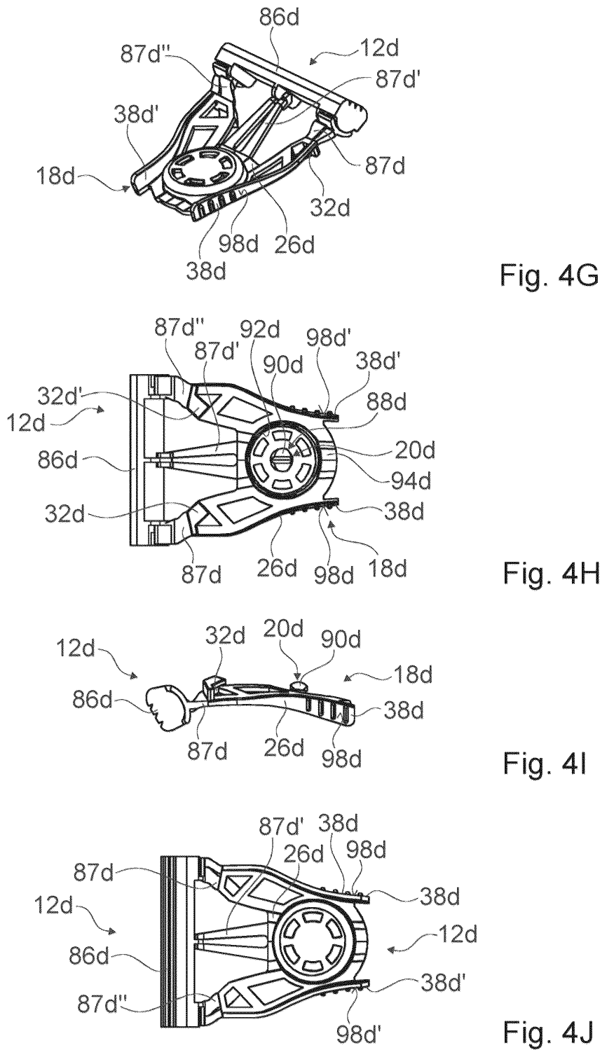

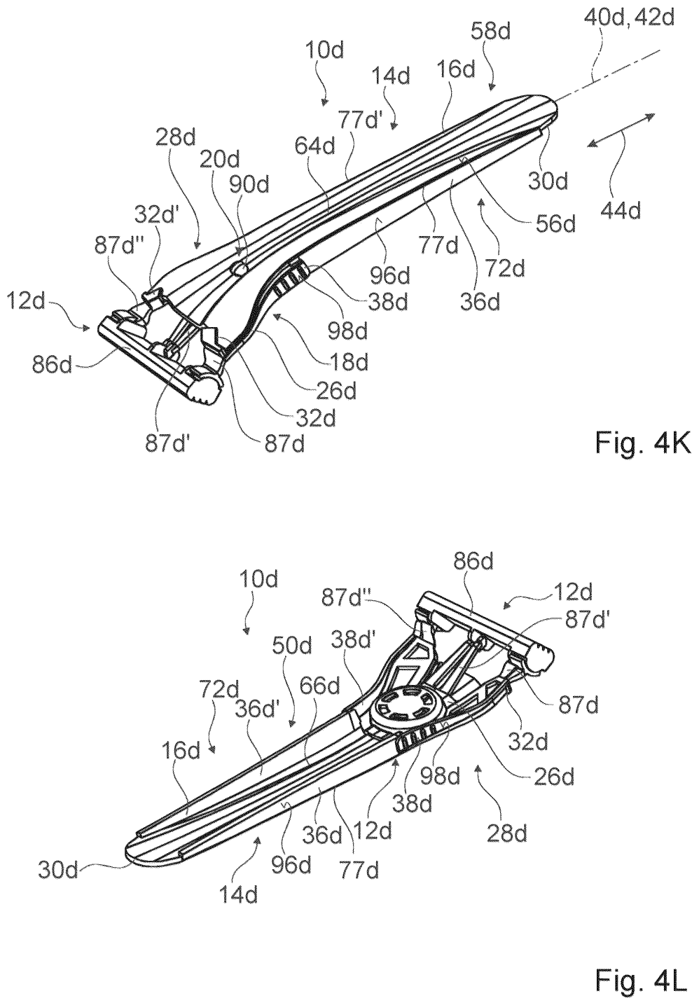

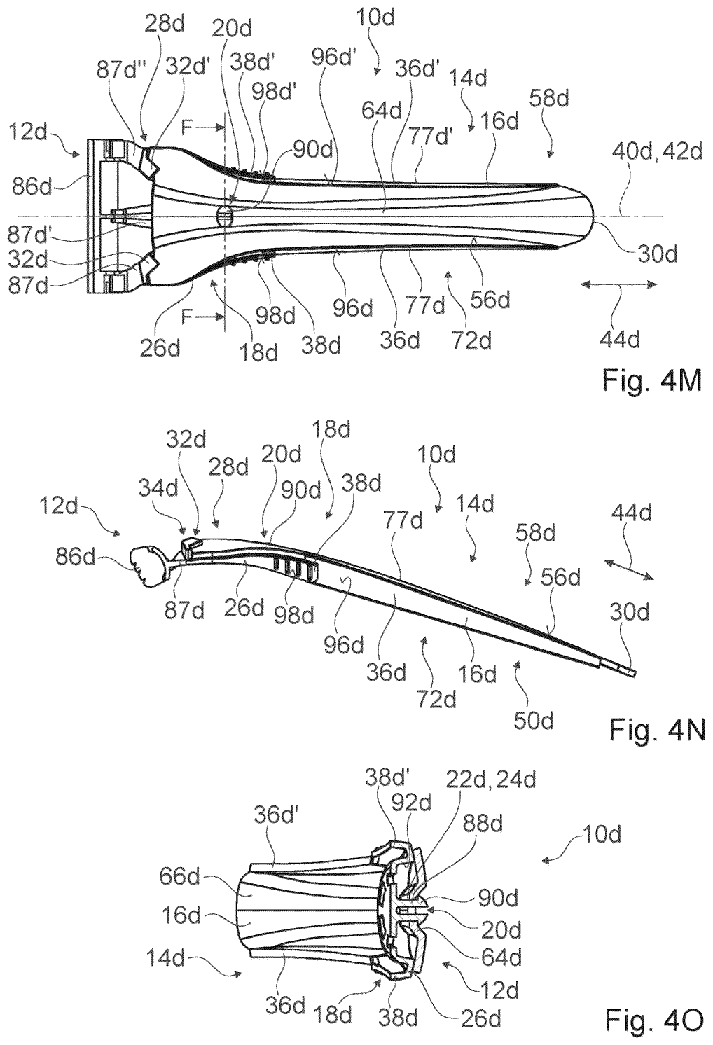

In the following, reference is made to FIGS. 4A to 4O, which show different views and manufacturing stages of the body care product 10d. Due to the different views, some elements are not shown in all the figures and are accordingly not in all the figures provided with reference numerals.

FIG. 4A shows a handle unit 14d of a body care product 10d in a schematic perspective illustration. The body care product 10d is in the present case realized as a razor.

The body care product 10d comprises an application unit 12d. The body care product 10d further comprises a handle unit 14d, which has a material handle body 16d. The body care product 10d also comprises a connection unit 18d, which connects the application unit 12d with the handle unit 14d.

The application unit 12d forms a shaving head of the body care product 10d. The application unit 12d in particular comprises a base body 26d, which is realized as a blade carrier. The base body 26d of the application unit 12d is completely made of a hard component. However, it would also be conceivable that the base body 26d of the application unit 12d is formed of a hard component and a soft component. The base body 26d of the application unit 12d implements a stationary blade carrier, which is configured for a movable, in particular pivotable, accommodation of a blade head 86d. The application unit 12d comprises the blade head 86d, which is arranged at the base body 26d. The base body 26d has three articulated arms 87d, 87d′, 87d″, which are configured for a pivotable accommodation of the blade head 86d. The blade head 86d may in particular be exchangeably connected with the base body 26d.

The handle unit 14d is configured to be gripped by the operator. The handle unit 14d comprises the material handle body 16d. The material handle body 16d of the handle unit 14d comprises a head region 28d. Furthermore, the material handle body 16d of the handle unit 14d comprises a handle region 72d. The head region 28d is configured for a connection of the application unit 12d. The handle unit 14d comprises at least one handle surface 56d, which forms a surface of the material handle body 16d. The handle surface 56d extends on a front side 58d from the head region 28d of the handle body 16d to the lower end of the body care product 10d at the bottom side. The handle surface 56d extends over the handle region 72d of the material handle body 16d. The handle surface 56d forms a partially cambered and/or partially convex shape on the front side 58d along the longitudinal axis 40d. The cambered shape is realized in an inverted fashion on the rear side 50d along the longitudinal axis 40d. On the rear side 50d the handle body 16d is partially concavely shaped in a region of the handle surface 56d. In a plan view, the handle body 16d has an I-shaped basic shape in a region of the handle surface 56d. In a plan view, the entire handle body 16d is in particular partially Y-shaped or T-shaped. In order to create the cambered convex shape, the handle body 16d is three-dimensionally deformed. The material handle body 16d of the handle unit 14d further comprises two stabilizing walls 36d, 36d′, which are angled with respect to a main extension plane of the handle unit 14d and extend substantially parallel to a longitudinal axis 40d of the handle unit 14d. The stabilizing walls 36d, 36d′ are in each case angled over a folding edge 77d, 77d′ with respect to a remaining portion of the material handle body 16d. A middle region of the material handle body 16d is arranged between the stabilizing walls 36d, 36d′. The stabilizing walls 36d, 36d′ in each case extend over the handle region 72d of the material handle body 16d. The stabilizing walls 36d, 36d′ are arranged on an outer edge of the material handle body 16d which extends substantially parallel to the longitudinal axis 40d. The stabilizing walls 36d, 36d′ are angled, perpendicular to the longitudinal axis 40d of the handle unit 14d, at an angle of at least 60°, preferably at least 80°, with respect to a main extension plane of the handle body 16d. The stabilizing walls 36d, 36d′ in each case form a free outer edge of the material handle body 16d.

The stabilizing walls 36d, 36d′ moreover in each case form a holding surface 96d, 96d′. For this purpose, the holding surfaces 96d, 96d′ are formed laterally on the outside of the stabilizing walls 36d, 36d′. The holding surfaces 96d, 96d′ are in particular configured to facilitate holding.

The handle body 16d further has a substantially constant material thickness. The handle body 16d moreover comprises a groove 64d, which extends on the front side 58d from the application unit 12d over the handle surface 56d as far as a bottom side of the handle unit 14d. The groove 64d runs centrally in parallel to the longitudinal axis 40d of the handle unit 14d. Along its extension parallel to the longitudinal axis 40d, the groove 64d has an at least approximately constant cross section, wherein the groove 64d slightly widens towards the application unit 12d. The groove 64d has a trapezoidal cross section, which widens towards the front side 58d. On the rear side 50d of the material handle body 16d, a web-shaped elevation 66d is formed that corresponds to the groove 64d. The web-shaped elevation 66d is here in particular formed during production of the groove 64d due to the constant material thickness.

The material handle body 16d is at least largely made of a paper material. The material handle body 16d consists completely of a paper material. The material handle body 16d of the handle unit 14d comprises at least one layer 30d of a paper material, which is formed three-dimensionally. The material handle body 16d consists of precisely one layer 30d of a paper material; however, a multilayer structure would also be conceivable.

The body care product 10d furthermore comprises the connection unit 18d. The connection unit 18d is configured for a form-fitting connection to the material handle body 16d of the handle unit 14d.

The connection unit 18d comprises a form-fitting element 20d. The form-fitting element 20d is configured to engage in a recess 22d of the application unit 12d and/or of the handle unit 14d. The form-fitting element 20d is configured to engage in a recess 22d of the handle unit 14d. The recess 22d of the handle unit 14d is realized as a pass-through recess 24d. The material handle body 16d delimits the pass-through recess 24d. The pass-through recess 24d is introduced in the head region 28d of the material handle body 16d. The pass-through recess 24d is arranged in the groove 64d. The pass-through recess 24d is configured to receive the form-fitting element 20d of the connection unit 18d. The pass-through recess 24d is in each case realized as a circular-cylindrical recess 22d. The form-fitting element 20d likewise has a cylindrical basic shape. The form-fitting element 20d is realized as a latching element. The latching element is realized in a mushroom shape with a stem section 88d and a head section 90d. Furthermore, the latching element is divided such that there are two springy sub-elements. The form-fitting element 20d is arranged in a cylindrical depression 92d of the connection unit 18d. The depression 92d enables an elastic deflection of the form-fitting element 20d during a connection of the connection unit 18d to the handle unit 14d. The form-fitting element 20d is configured to clamp the handle body 16d against a contact surface 102d of the base body 26d. The contact surface 102d is in particular realized as a surface which corresponds to the underside of the head region 28d of the handle body 16d and which the handle body 16d can adjoin over a large surface area.

The material handle body 16d of the handle unit 14d has in the head region 28d on the rear side 50d the elevation 66d, which is configured for a positioning of the connection unit 18d relative to the handle unit 14d. The elevation 66d is configured to engage into a depression 94d of the connection unit 18d.

The connection unit 18d furthermore comprises at least one receiving groove 32d, 32d′. The connection unit 18d comprises two receiving grooves 32d, 32d′. The receiving grooves 32d, 32d′ are arranged on the outer articulated arms 87d, 87d″ of the base body 26d of the application unit 12d. The receiving grooves 32d, 32d′ are each configured to receive a front edge 34d of the material handle body 16d of the handle unit 14d, which faces towards the application unit 12d. The handle body 16d in particular has a Y-shaped basic shape, wherein in particular the two outer front edges are received in the receiving grooves 32d, 32d′.

The receiving grooves 32d, 32d′ are configured for a clamping accommodation at least of an edge of the material handle body 16d. The receiving grooves 32d, 32d′ extend substantially perpendicular to a longitudinal axis 40d of the handle unit 14d. It is conceivable that for example barbs are provided in the receiving grooves 32d, 32d′, which improve a clamping. The receiving grooves 32d, 32d′ are configured to provide a clamping connection to the material handle body 16d. Herein the receiving grooves 32d, 32d′ are made of plastic, such that a plastic of the connection unit 18d is arranged on the outside, which receives a paper material of the handle unit 14d on an inside. The receiving grooves 32d, 32d′ in each case form a U-shaped groove, said grooves being open towards the handle unit 14d.

During production, the handle unit 14d and the connection unit 18d are provided. Then the handle unit 14d and the connection unit 18d are brought together, wherein the front edge of the material handle body 16d is pushed into the receiving grooves 32d, 32d′ in a slightly angled fashion. After this the material handle body 16d is pivoted into an end position, wherein the form-fitting element 20d extends from the rear side 50d through the pass-through recess 24d and latches on the front side 58d of the handle unit 14d with the handle body 16d by means of the head section 90d.

The connection unit 18d furthermore comprises at least one delimiting wall 38d, 38d′. The connection unit 18d comprises two delimiting walls 38d, 38d′. The delimiting walls 38d, 38d′ are angled with respect to a main extension plane of the connection unit 18d and extend substantially parallel to a longitudinal axis 40d of the handle unit 14d. The delimiting walls 38d, 38d′ extend from the receiving grooves 32d, 32d′ towards the handle unit 14d. The delimiting walls 38d, 38d′ are arranged on an outer edge of the connection unit 18d, in particular of the base body 26d of the application unit 12d, which extends substantially parallel to the longitudinal axis 42d. The delimiting walls 38d, 38d′ are angled, perpendicular to the longitudinal axis of the connection unit 18d, at an angle of at least 60°, preferably at least 80°, with respect to a main extension plane of the connection unit 18d. The delimiting walls 38d, 38d′ in each case form a free outer edge of the material handle body 16d. The delimiting walls 38d, 38d′ furthermore each protrude towards the bottom side. The delimiting walls 38d, 38d′ are configured for an at least partial delimitation and stabilization of the stabilizing walls 36d, 36d′ of the material handle body 16d. For this purpose, the stabilizing walls 36d, 36d′ bear, in an assembled state, on the inside against the delimiting walls 38d, 38d′ in a region facing towards the application unit 12d. The holding surfaces 96d, 96d′ on the stabilizing walls 36d, 36d′ continue in holding surfaces 98d, 98d′ on the delimiting walls 38d, 38d′ of the connection unit 18d.

The application unit 12d comprises the base body 26d. The base body 26d is fixedly connected with the connection unit 18d. The base body 26d is integrally connected with the connection unit 18d. The base body 26d is realized in a one-part implementation with the connection unit 18d. The connection unit 18d is integrated in the base body 26d. The form-fitting element 20d, the receiving grooves 32d, 32d′ and the delimiting walls 38d, 38d′ are realized integrally with the base body 26d of the application unit 12d. The form-fitting element 20d, the receiving grooves 32d, 32d′ and the delimiting walls 38d, 38d′ are realized in a one-part implementation with the base body 26d of the application unit 12d. The depression 94d is likewise realized in a one-part implementation with the base body 26d of the application unit 12d.

Alternatively, it would also be conceivable that the application unit 12d comprises at least one base body 26d which is exchangeably connected with the connection unit 18d. In such a case it would in particular be conceivable that the entire application unit 12d is realized, for example, as an exchangeable blade.

The body care product 10d has a longitudinal axis 42d, a height axis and a width axis. The longitudinal axis 42d is arranged in parallel to a main extension direction 44d of the body care product 10d. If the body care product 10d is placed with a rear side 50d on a flat surface, such that the longitudinal axis 42d is arranged in parallel to the surface, the height axis is arranged perpendicular to the longitudinal axis 42d and perpendicular to the surface and to the width axis. The width axis is arranged perpendicular to the longitudinal axis 42d and perpendicular to the height axis. In the present case, the body care product 10d has a length, in particular parallel to the longitudinal axis 42d and measured parallel to the bearing surface, of 100 mm to 160 mm, preferably of 130 mm to 150 mm. The body care product 10d further has a maximum height, in particular measured parallel to the height axis and perpendicular to the bearing surface, of 10 mm to 25 mm and preferably of 12 mm to 18 mm. The body care product 10d further has in a point of maximum width a width, in particular parallel to the width axis, of 25 mm to 50 mm and preferably of 30 mm to 45 mm.

The structure of the body care product 10d is provided with a number of lays and a number of layers, as mentioned in the description.

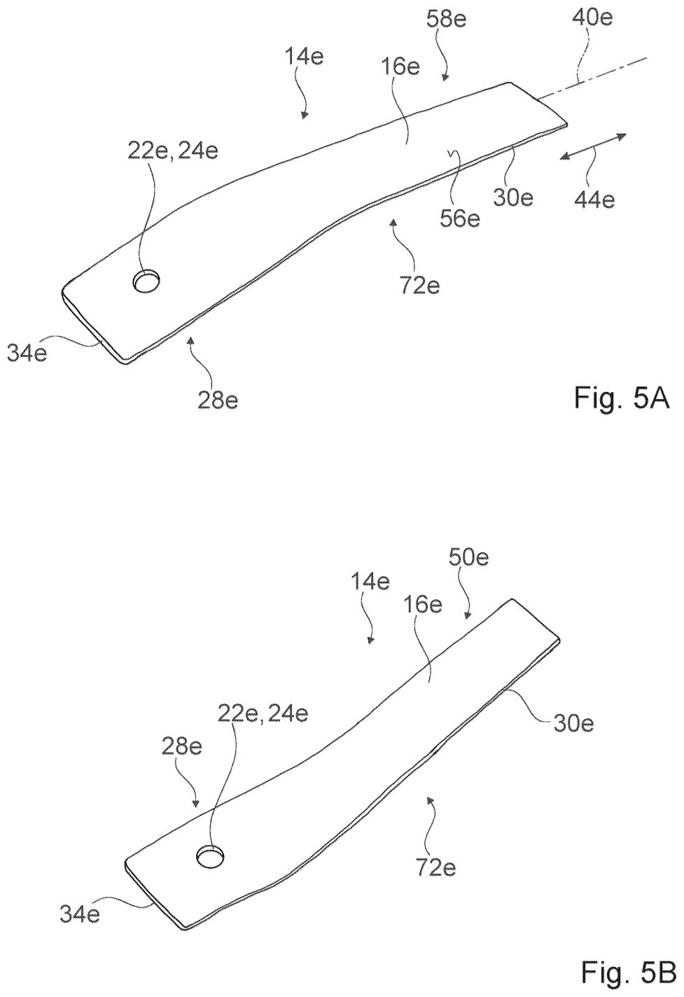

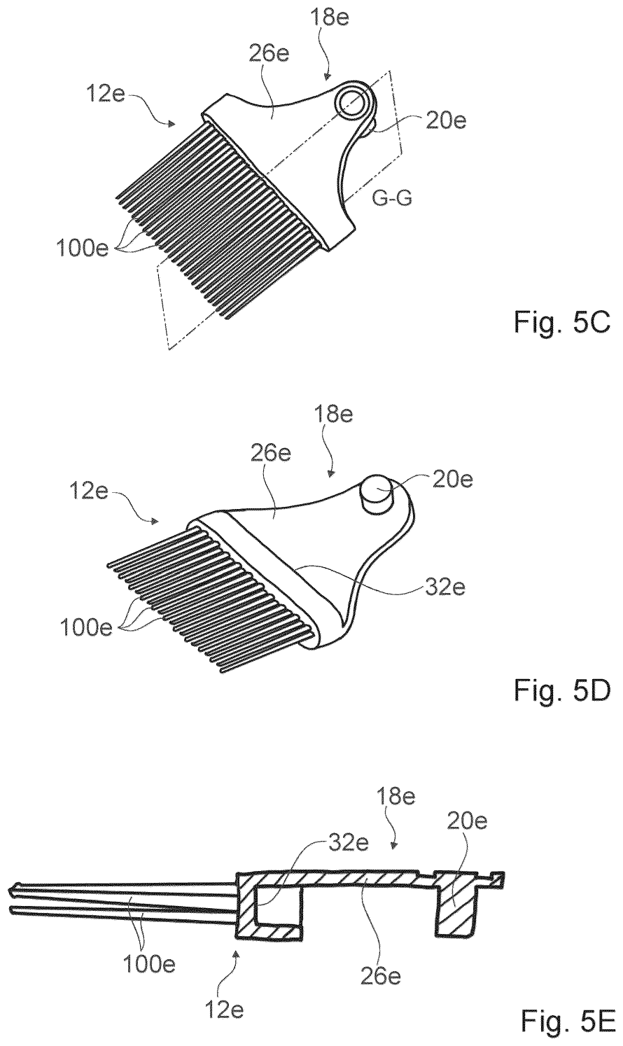

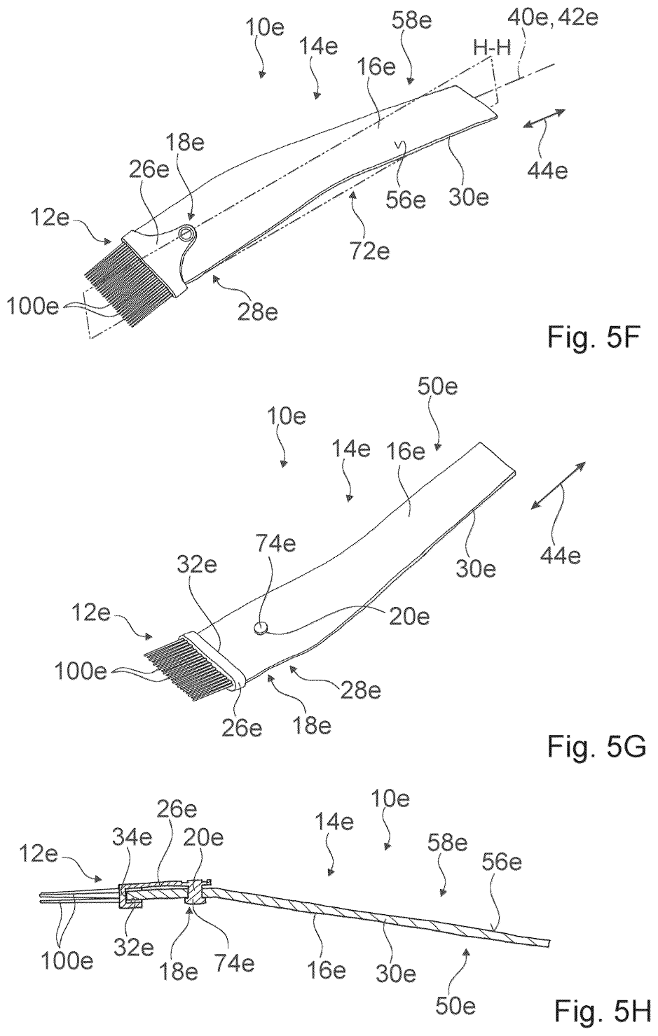

In the following, reference is made to FIGS. 5A to 5H, which show different views and manufacturing stages of the body care product 10e. Due to the different views, some elements are not shown in all the figures and are accordingly not in all the figures provided with reference numerals.

FIG. 5A shows a handle unit 14e of a body care product 10e in a schematic perspective illustration. The body care product 10e is in the present case realized as a brush, in particular as an application brush.

The body care product 10e comprises an application unit 12e. The body care product 10e furthermore comprises a handle unit 14e, which has a material handle body 16e. The body care product 10e also comprises a connection unit 18e, which connects the application unit 12e with the handle unit 14e.

The application unit 12e forms a brush head of the body care product 10e. The application unit 12e in particular has a base body 26e that is realized as a brush-hair carrier. A base body 26e of the application unit 12e is completely made of a hard component. However, it would also be conceivable that the base body 26e of the application unit 12e is made of a hard component and a soft component. The application unit 12e further comprises several brush bristles 100e, which are arranged on the base body 26e. The brush bristles 100e are injection-molded to the base body 26e on an upper side.

The handle unit 14e is configured to be gripped by the operator. The handle unit 14e comprises the material handle body 16e. The material handle body 16e of the handle unit 14e comprises a head region 28e. The material handle body 16e of the handle unit 14e further comprises a handle region 72e. The head region 28e is configured for a connection of the application unit 12e. The handle unit 14e comprises at least one handle surface 56e, which forms a surface of the material handle body 16e. The handle surface 56e extends on a front side 58e from the head region 28e of the handle body 16e to the lower end of the body care product 10e at the bottom side. The handle surface 56e extends over the handle region 72e of the material handle body 16e. The handle surface 56e forms a partially bent shape on the front side 58e along the longitudinal axis 42e. The bent shape is formed in an inverted fashion on the rear side 50e along the longitudinal axis 42e. By way of example, the handle body 16e has a rectangular basic shape. The handle body 16e further has a substantially constant material thickness.

The material handle body 16e is at least largely made of a paper material. The material handle body 16e consists completely of a paper material. The material handle body 16e of the handle unit 14e comprises at least one layer 30e of a paper material, which is formed three-dimensionally. The material handle body 16e consists of precisely one layer 30e of a paper material.

The body care product 10e furthermore comprises the connection unit 18e. The connection unit 18e is configured for a form-fitting connection to the material handle body 16e of the handle unit 14e.

The connection unit 18e comprises a form-fitting element 20e. The form-fitting element 20e is configured to engage in a recess 22e of the application unit 12e and/or of the handle unit 14e. The form-fitting element 20e is configured to engage in a recess 22e of the handle unit 14e. The recess 22e of the handle unit 14e is realized as a pass-through recess 24e. The material handle body 16e delimits the pass-through recess 24e. The pass-through recess 24e is introduced in the head region 28e of the material handle body 16e. The pass-through recess 24e is configured for receiving the form-fitting element 20e of the connection unit 18e. The pass-through recess 24e is in each case realized as a circular-cylindrical recess 22e. The form-fitting element 20e likewise has a cylindrical basic shape. The form-fitting element 20e is realized as a rivet. The form-fitting element 20e is realized as a plastic rivet. In a state when it is connected to the handle unit 14e, the form-fitting element 20e has a shaft and a form-fitting head 74e, 74e′. The form-fitting head 74e, 74e′ of the form-fitting element 20e has a larger diameter than the pass-through recess 24e and is arranged on a rear side 50e of the handle unit 14e, which faces away from the application unit 12e. The form-fitting head 74e, 74e′ of the form-fitting element 20e prevents a retraction of the connection unit 18e. The form-fitting element 20e is arranged on a triangular prolongation of the connection unit 18e, which extends towards a bottom side.

The connection unit 18e further comprises at least one receiving groove 32e. The receiving groove 32e is arranged in a region of the brush bristles 100e. The receiving groove 32e is configured to receive a front edge 34e of the material handle body 16e of the handle unit 14e, which faces towards the application unit 12e. The receiving groove 32e is configured for a clamping accommodation at least of an edge of the material handle body 16e. The receiving groove 32e extends substantially perpendicular to a longitudinal axis 40e of the handle unit 14e. It is conceivable that for example barbs are provided in the receiving groove 32e, which improve a clamping. The receiving groove 32e is configured to provide a clamping connection to the material handle body 16e. Herein the receiving groove 32e is made of plastic, such that a plastic of the connection unit 18e is arranged on the outside, which receives a paper material of the handle unit 14e on the inside. The receiving groove 32e is here realized as a U-shaped groove, which is open towards the handle unit 14e.

During production, the handle unit 14e and the connection unit 18e are provided. Then the handle unit 14e and the connection unit 18e are brought together, wherein the front edge 34e of the material handle body 16e is pushed into the receiving groove 32e in a slightly angled fashion. After this the material handle body 16e is pivoted into an end position, wherein the form-fitting element 20e extends through the pass-through recess 24e. Following this, the penetrating shaft of the form-fitting element 20e is deformed. Herein various deformation possibilities, deemed expedient by someone skilled in the art, are conceivable, such as for example by pressure, with the form-fitting element 20e being pressed and/or slightly crushed; or by pressure and heat, with the form-fitting element 20e being deformed and/or slightly crushed by means of a heated punch. Herein the pressure is in particular selected in such a way that a controlled crushing takes place. When the pressure is applied, in particular an expansion of the compressed form-fitting element 20e is controlled. Preferably a punch intended for this purpose is in particular not flat but concavely curved. A round shape of the touch surface in particular controls the flowing of the material of the form-fitting element 20e.

The application unit 12e comprises the base body 26e. The base body 26e is fixedly connected with the connection unit 18e. The base body 26e is integrally connected with the connection unit 18e. The base body 26e is realized in a one-part implementation with the connection unit 18e. The connection unit 18e is integrated in the base body 26e. The form-fitting element 20e and the receiving groove 32e are realized integrally with the base body 26e of the application unit 12e. The form-fitting element 20e and the receiving groove 32e are realized in a one-part implementation with the base body 26e of the application unit 12e.

The body care product 10e has a longitudinal axis 42e, a height axis and a width axis. The longitudinal axis 42e is arranged in parallel to a main extension direction 44e of the body care product 10e. If the body care product 10e is placed with a rear side 50e on a flat surface, such that the longitudinal axis 42e is arranged in parallel to the surface, the height axis is arranged perpendicular to the longitudinal axis 42e and perpendicular to the surface and to the width axis. The width axis is arranged perpendicular to the longitudinal axis 42e and perpendicular to the height axis. In the present case, the body care product 10e has a length, in particular parallel to the longitudinal axis 42e and measured parallel to the bearing surface, of 100 mm to 150 mm, preferably of 125 mm to 145 mm. The body care product 10e furthermore has a maximum height, in particular measured parallel to the height axis and perpendicular to the bearing surface, of 10 mm to 25 mm and preferably of 12 mm to 18 mm. The body care product 10e further has in a point of maximum width a width, in particular parallel to the width axis, of 15 mm to 35 mm, preferably of 20 mm to 40 mm.

The structure of the body care product 10e is provided with a number of lays and a number of layers, as mentioned in the description.

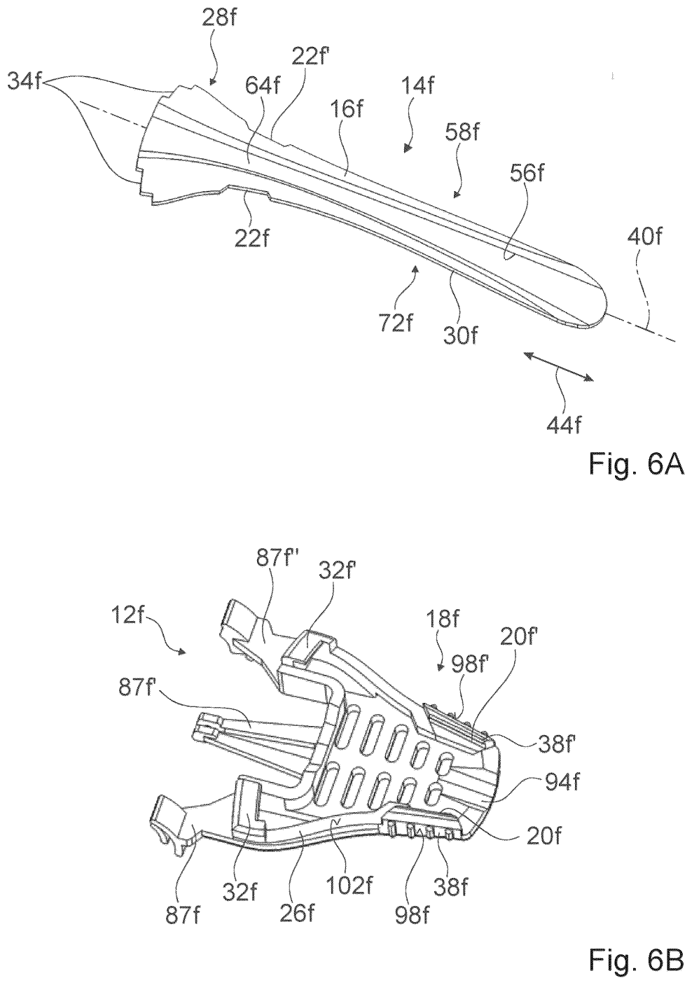

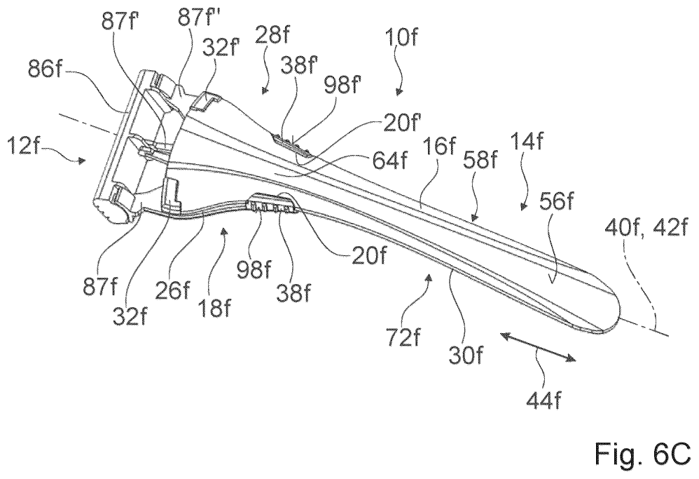

In the following, reference is made to FIGS. 6A to 6C, which show different views and manufacturing stages of the body care product 10f. Due to the different views, some elements are not shown in all the figures and are accordingly not in all the figures provided with reference numerals.

FIG. 6A shows a handle unit 14f of a body care product 10f in a schematic perspective illustration. The body care product 10f is in the present case realized as a razor.

The body care product 10f comprises an application unit 12f. The body care product 10f further comprises a handle unit 14f, which has a material handle body 16f. The body care product 10f moreover comprises a connection unit 18f, which connects the application unit 12f with the handle unit 14f.

The application unit 12f forms a shaving head of the body care product 10f. The application unit 12f in particular comprises a base body 26f, which is realized as a blade carrier. The base body 26f of the application unit 12f is completely made of a hard component. However, it would also be conceivable that the base body 26f of the application unit 12f is formed of a hard component and a soft component. The base body 26f of the application unit 12f forms a stationary blade carrier, which is configured for a movable, in particular pivotable, accommodation of a blade head 86f. The application unit 12f comprises the blade head 86f, which is arranged at the base body 26f. The base body 26f comprises three articulated arms 87f, 87f′, 87f″, which are configured for a pivotable accommodation of the blade head 86f. The blade head 86f may in particular be exchangeably connected with the base body 26f.

The handle unit 14f is configured to be gripped by the operator. The handle unit 14f comprises the material handle body 16f. The material handle body 16f of the handle unit 14f comprises a head region 28f. The material handle body 16f of the handle unit 14f further comprises a handle region 72f. The head region 28f is configured for a connection of the application unit 12f. The handle unit 14f comprises at least one handle surface 56f, which forms a surface of the material handle body 16f. The handle surface 56f extends on a front side 58f from the head region 28f of the handle body 16f to the lower end of the body care product 10f at the bottom side. The handle surface 56f extends over the handle region 72f of the material handle body 16f. The handle surface 56f forms a partially cambered and/or partially convex shape on the front side 58f along a longitudinal axis 40f. The cambered shape is formed in an inverted fashion on a rear side along the longitudinal axis 40f. On the rear side 50f, the handle body 16f is partly concavely shaped in a region of the handle surface 56f. In a plan view, the handle body 16f has an I-shaped basic shape in a region of the handle surface 56f. In a plan view, the entire handle body 16f is in particular partially Y-shaped or T-shaped. In order to create the cambered convex shape, the handle body 16f is three-dimensionally deformed.

The handle body 16f further has a substantially constant material thickness. The handle body 16f moreover comprises a groove 64f, which extends on the front side 58f from the application unit 12f over the handle surface 56f as far as a bottom side of the handle unit 14f. The groove 64f runs centrally in parallel to the longitudinal axis 40f of the handle unit 14f. Along its extension parallel to the longitudinal axis 40f, the groove 64f has an at least approximately constant cross section, wherein the groove 64f slightly widens towards the application unit 12f. The groove 64f has a trapezoidal cross section, which widens towards the front side 58f. On the rear side 50f of the material handle body 16f, a web-shaped elevation is formed that corresponds to the groove 64f. The web-shaped elevation is here in particular formed during production of the groove 64f due to the constant material thickness.

The material handle body 16f is at least largely made of a paper material. The material handle body 16f consists completely of a paper material. The material handle body 16f of the handle unit 14f comprises at least one layer 30f of a paper material, which is formed three-dimensionally. The material handle body 16f consists of precisely one layer 30f of a paper material; however, a multilayer structure would also be conceivable.

The body care product 10f further comprises the connection unit 18f. The connection unit 18f is configured for a form-fitting connection to the material handle body 16f of the handle unit 14f.

The connection unit 18f comprises respectively one form-fitting element 20f, 20f′ on each delimiting wall 38f, 38f′. The form-fitting elements 20f, 20f′ are configured to clamp the handle body 16f against the contact surface 102f of the base body 26f. The contact surface 102f is in particular realized as a surface which corresponds to the underside of the head region 28f of the handle body 16f and which the handle body 16f can adjoin over a large surface area. The contact surface 102f is realized in a peripheral region of the base body 26f. The form-fitting elements 20f, 20f′ are configured to engage around the handle body 16f. The form-fitting elements 20f, 20f′ are formed on the delimiting walls 38f, 38f′ as projecting edges and are thus supported in a slightly springy manner. The edges are substantially oriented in the direction of the longitudinal axis 42f of the body care product. The form-fitting elements 20f, 20f′ are configured to partially engage in recesses 22f, 22f′ of the handle body 16f. The recesses 22f, 22f′ of the handle body 16f are realized as lateral indentations.

The material handle body 16f of the handle unit 14f has in the head region 28f on the rear side 50f the elevation 66f, which is configured for a positioning of the connection unit 18f relative to the handle unit 14f. The elevation 66f is configured to engage into a depression 94f of the connection unit 18f.

The connection unit 18f furthermore comprises at least one receiving groove 32f, 32f′. The connection unit 18f comprises two receiving grooves 32f, 32f′. The receiving grooves 32f, 32f′ are arranged on the outer articulated arms 87f, 87f″ of the base body 26f of the application unit 12f. The receiving grooves 32f, 32f′ are each configured to receive a front edge 34f of the material handle body 16f of the handle unit 14f, which faces towards the application unit 12f. The handle body 16f in particular has a Y-shaped basic shape, wherein in particular the two outer front edges 34f are received in the receiving grooves 32f, 32f′.

The receiving grooves 32f, 32f′ are configured for a clamping accommodation at least of an edge of the material handle body 16f. The receiving grooves 32f, 32f′ extend substantially perpendicular to a longitudinal axis 40f of the handle unit 14f. It is conceivable that for example barbs are provided in the receiving grooves 32f, 32f′, which improve a clamping. The receiving grooves 32f, 32f′ are configured to provide a clamping connection to the material handle body 16f. Herein the receiving grooves 32f, 32f′ are made of plastic, such that a plastic of the connection unit 18f is arranged on the outside, which receives a paper material of the handle unit 14f on the inside. The receiving grooves 32f, 32f′ in each case form a U-shaped groove, said grooves being open towards the handle unit 14f.

During production, the handle unit 14f and the connection unit 18f are provided. Then the handle unit 14f and the connection unit 18f are brought together, wherein the front edge of the material handle body 16f is pushed into the receiving grooves 32f, 32f′ in a slightly angled fashion. After this the material handle body 16f is pivoted into an end position, wherein the form-fitting elements 20f, 20f′ then clamp the handle body 16f and thus latch.

The connection unit 18f furthermore comprises at least one delimiting wall 38f, 38f′. The connection unit 18f comprises two delimiting walls 38f, 38f′. The delimiting walls 38f, 38f′ are angled with respect to a main extension plane of the connection unit 18f and extend substantially parallel to the longitudinal axis 40f of the handle unit 14f. The delimiting walls 38f, 38f′ extend from the receiving grooves 32f, 32f′ towards the handle unit 14f. The delimiting walls 38f, 38f′ are arranged on an outer edge of the connection unit 18f, in particular of the base body 26f of the application unit 12f, which extends substantially parallel to the longitudinal axis 42f. The delimiting walls 38f, 38f′ are angled, perpendicular to the longitudinal axis of the connection unit 18f, at an angle of at least 60°, preferably at least 80°, with respect to a main extension plane of the connection unit 18f. The delimiting walls 38f, 38f′ in each case form a free outer edge of the material handle body 16f. The delimiting walls 38f, 38f′ furthermore each protrude towards the bottom side. Holding surfaces 98f, 98f′ are formed on the delimiting walls 38f, 38f′ of the connection unit 18f.

The application unit 12f comprises the base body 26f. The base body 26f is fixedly connected with the connection unit 18f. The base body 26f is integrally connected with the connection unit 18f. The base body 26f is realized in a one-part implementation with the connection unit 18f. The connection unit 18f is integrated in the base body 26f. The form-fitting elements 20f, 20f′, the receiving grooves 32f, 32f′ and the delimiting walls 38f, 38f′ are realized integrally with the base body 26f of the application unit 12f. The form-fitting elements 20f, 20f′, the receiving grooves 32f, 32f′ and the delimiting walls 38f, 38f′ are realized in a one-part implementation with the base body 26f of the application unit 12f. The depression 94f is likewise realized in a one-part implementation with the base body 26f of the application unit 12f.

Alternatively, it would also be conceivable that the application unit 12f comprises at least one base body 26f, which is exchangeably connected with the connection unit 18f. Herein it would in particular be conceivable that the entire application unit 12f is realized, for example, as an exchangeable blade.

The body care product 10f has a longitudinal axis 42f, a height axis and a width axis. The longitudinal axis 42f is arranged in parallel to a main extension direction 44f of the body care product 10f. If the body care product 10f is placed with its rear side on a flat surface, such that the longitudinal axis 42f is arranged in parallel to the surface, the height axis is arranged perpendicular to the longitudinal axis 42f and perpendicular to the surface and to the width axis. The width axis is arranged perpendicular to the longitudinal axis 42f and perpendicular to the height axis. In the present case, the body care product 10f has a length, in particular parallel to the longitudinal axis 42f and measured parallel to the bearing surface, of 100 mm to 160 mm, preferably of 130 mm to 150 mm. The body care product 10f furthermore has a maximum height, in particular measured parallel to the height axis and perpendicular to the bearing surface, of 10 mm to 25 mm and preferably of 12 mm to 18 mm. The body care product 10f further has in a point of maximum width a width, in particular parallel to the width axis, of 25 mm to 50 mm and preferably of 30 mm to 45 mm.

The structure of the body care product 10f is provided with a number of lays and a number of layers, as mentioned in the description.

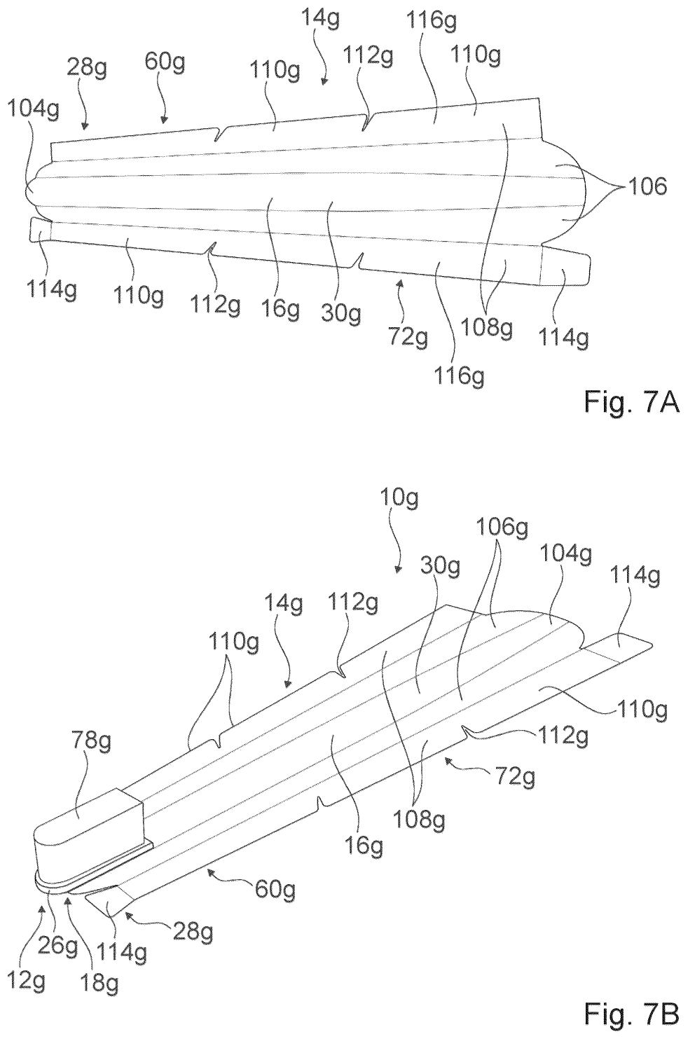

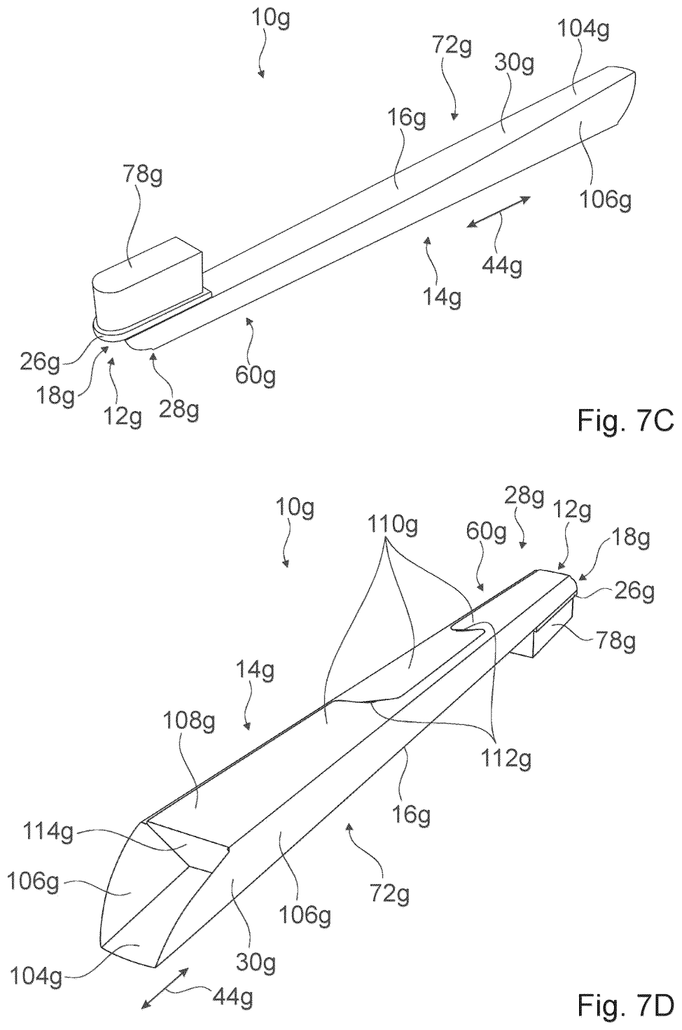

In the following, reference is made to FIGS. 7A to 7D, which show different views and manufacturing stages of the body care product 10g. Due to the different views, some elements are not shown in all the figures and are accordingly not in all the figures provided with reference numerals.

FIGS. 7C and 7D respectively show the finished body care product 10g. FIGS. 7C and 7D show a handle unit 14g of a body care product 10g with an application unit 12g in a schematic perspective illustration. The body care product 10g is realized as an oral hygiene means. The body care product 10g is in the present case realized as a toothbrush.

The body care product 10g comprises the application unit 12g. The body care product 10g further comprises the handle unit 14g, which has a material handle body 16g. The body care product 10g also comprises a connection unit 18g, which connects the application unit 12g with the handle unit 14g.

The body care product 10g has a longitudinal axis, a height axis and a width axis. The longitudinal axis 42g is arranged in parallel to a main extension direction 44g of the body care product 10g. If the body care product 10g is placed with its rear side on a flat surface, such that the longitudinal axis 42g is arranged in parallel to the surface, the height axis is arranged perpendicular to the longitudinal axis 42g and perpendicular to the surface and to the width axis. The width axis is arranged perpendicular to the longitudinal axis 42g and perpendicular to the height axis. In the present case, the body care product 10g has a length, in particular parallel to the longitudinal axis 42g and measured parallel to the bearing surface, of 140 mm to 210 mm and preferably of 165 mm to 185 mm. The body care product 10g furthermore has a maximum height, in particular measured parallel to the height axis and perpendicular to the bearing surface, of 12 mm to 25 mm and preferably of 15 mm to 19 mm. The body care product 10g further has in a point of maximum width a width, in particular parallel to the width axis, of 10 mm to 25 mm and preferably of 13 mm to 18 mm.

The application unit 12g forms a brush head of the body care product 10g. In the present case, the application unit 12g is realized as a toothbrush head. The application unit 12g in particular comprises a base body 26g, which is realized as a bristle carrier. The base body 26g forms a connection to the connection unit 18g. The base body 26g of the application unit 12g forms a bristle carrier. The application unit 12g further comprises several bristles 78g arranged on the base body 26g. The bristles 78g are injection-molded to the base body 26g. The bristles 78g consist of a material for injection-molded bristles; this material also forms the base body 26g.

The handle unit 14g is configured to be gripped by the operator. The handle unit 14g comprises the material handle body 16g. The material handle body 16g of the handle unit 14g comprises a head region 28g. Beyond this, the material handle body 16g of the handle unit 14g comprises a handle region 72g and a neck region 60g that is arranged between the handle region 72g and the head region 28g. The head region 28g is configured for a connection of the application unit 12g. The handle unit 14g comprises at least one handle surface, which forms a surface of the material handle body 16g. The handle body 16g is folded from the non-folded state, depicted in FIG. 7A, respectively FIG. 7B, to form a three-dimensional body. In a folded state, the handle body 16g has a rectangular or trapezoidal cross section, which is composed of four sides of the base body 16g. In a folded state, the base body 16g is realized hollow. The base body 16g in particular consists of a paper sheet, which may consist of several layers or sub-layers. The base body 16g has a front wall 104g, two side walls 106g and a rear wall 108g which is plugged together from several tabs 110g. Moreover, two end tabs 114g are arranged at the rear wall 108g, which are configured to close an upper side and a bottom side of the base body 16g in a folded state. The end tabs 114g close a hollow space formed by the front wall 104g, the side walls 106g, 106g′ and the rear wall 108g on the upper side and on the bottom side.

In FIG. 7A a blank of the paper material for the handle unit 16g is shown. Here the application unit 12g has not yet been fastened to the blank. The tabs 110g of the base body 16g respectively adjoin outside-situated edges of the side walls. By way of example, the base body 16g has respectively three tabs 110g per side. The tabs 110g are partially separated by notches 112g. Two notches 112g, which respectively delimit the tabs 110g, are provided per long side. The tabs 110g are realized as plug-in tabs configured to be plugged into one another on the rear side via the notches 112g. During assembly, the notches 112g are slid into one another, such that the tabs 110g come to lie one above the other—one at the top, one at the bottom. This can be seen in FIG. 7D. Thus the sub-layers 116g of the rear wall 108g are fixed against one another.

In the folding around the folding axes, the sub-layers 116g of the front wall 104g, the side walls 106g, 106g′ and the rear wall 108g are folded against one another, such that they form a 3D body. A tetragonal cross-section is formed. Overall, not four but five sub-layers 116g are provided, such that an overlap of two of these sub-layers 116g can take place. The overlap takes place on the side on which the sub-layers 116g are fixed against one another. This cross-section is fixed to one another, for example, by gluing or by one or more tabs slid into one another. In the example shown, the sub-layers 116g of the rear wall 108g are plugged into one another. Due to their geometry and the geometry of the cross-section, the tabs 110g may be geometrically blocked in the final product such that in effect they cannot slip out.

Furthermore, the tabs 110g are designed in such a way that they are placed in a certain range around the double-layered rear wall 108g. Herein they bring about a further stabilization of the sub-layers 116g against one another.

The handle body 16g contains folding axes transversely to the main extension direction, for sub-layers 116g that (after the folding) close the inner space to a certain extent. Viewed in the main extension direction 44g, these sub-layers 116g-which are realized as end tabs 114g—are situated in the region of the end at which the application unit 12g is situated and at the end situated opposite the application unit 12g. These end tabs 114g close the inner space to a certain extent at the end of the application unit 12g and at the opposite end. The end tabs 114g are only “folded in” and are not fixed any further.

The material handle body 16g is at least largely made of a paper material. The material handle body 16g consists completely of a paper material. The material handle body 16g of the handle unit 14g comprises at least one layer 30g of a paper material, which is formed three-dimensionally. The material handle body 16g consists of precisely one layer 30g of a paper material.

The body care product 10g furthermore comprises the connection unit 18g. The connection unit 18g is configured for a material-bonding connection with the material handle body 16g of the handle unit 14g.

The connection unit 18g is connected to the handle body 16g with the rear side. The connection is preferably created by gluing or welding.

The application unit 12g comprises the base body 26g. The base body 26g is fixedly connected with the connection unit 18g. The base body 26g is integrally connected with the connection unit 18g. In the injection molding procedure, the base body 26g is in one step produced and connected to the connection unit 18g.

The structure of the body care product 10g is equipped with a number of lays and a number of layers, as mentioned in the description.

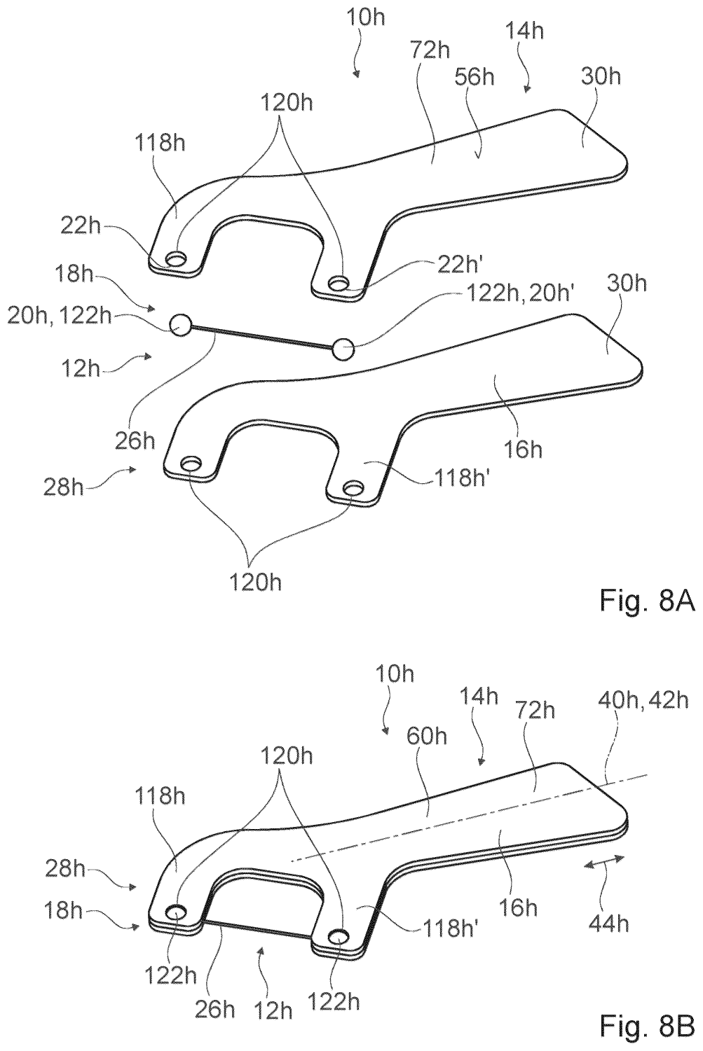

In the following, reference is made to FIGS. 8A and 8B, which show different views of an alternative body care product 10h according to the invention. Due to the different views, some elements are not shown in all the figures and are accordingly not in all the figures provided with reference numerals.

FIG. 8A shows a handle unit 14h of a body care product 10h in a schematic perspective illustration. The body care product 10h is realized as an oral hygiene means. The body care product 10h is in the present case realized as a flosser.

The body care product 10h comprises an application unit 12h. The body care product 10h further comprises the handle unit 14h, which has a material handle body 16h. The body care product 10h also comprises a connection unit 18h, which connects the application unit 12h with the handle unit 14h.

The application unit 12h forms a dental-floss thread of the body care product 10h. In the present case, the application unit 12h is embodied as a dental-floss thread. The application unit 12h in particular has a base body 26h that is realized as a thread. The base body 26h forms a connection to the connection unit 18h. The connection unit 18h is realized as a ball of plastic that is injection-molded to the dental-floss thread.

The handle unit 14h is configured to be gripped by the operator. The handle unit 14h comprises the material handle body 16h. The material handle body 16h of the handle unit 14h comprises a head region 28h. Beyond this, the material handle body 16h of the handle unit 14h comprises a handle region 72h and a neck region 60h that is arranged between the handle region 72h and the head region 28h. The head region 28h is configured for a connection of the application unit 12h. The head region 28h is realized as a U-shaped geometry with two legs, respectively arms. In a final state, the material handle body 16h has a flat basic shape that is towards the application unit 12h divided into two legs 118h, 118h′. The handle unit 14h comprises at least one handle surface 56h, which forms a surface of the material handle body 16h.

The material handle body 16h is at least largely made of a paper material. The material handle body 16h consists completely of a paper material. The material handle body 16h of the handle unit 14h may comprise at least one layer 30h of a paper material, which is formed three-dimensionally. The material handle body 16h consists of precisely one layer 30h of a paper material.

The head region 28h is formed as a U-shaped geometry with the two legs 118h, 118h′. The legs 118h, 118h′ are respectively provided with a through hole 120h, which is required for the anchoring of the application unit 12h. Respectively one through hole 120h is formed on the two legs 118h, and the through holes 120h are realized identically on both layers 30h which are later brought together.

The application unit 12h comprises two connection units 18h, which are implemented as balls 122h. The balls 122h are situated in the region of the ends of the single application unit 12h, where subsequently the connection to the handle body 16h is created. The balls 122h are laid into the through holes 120h of a layer 30h, the layer 30h is then connected to the second layer 30h by gluing, by sealing, by welding, etc. This takes place in such a manner that the balls 122h respectively get to lie in the through holes 120h of the further layer 30h. In the final product the balls 122h lie between the layers 30h in the through holes 120h. By this implementation, the application unit 12h, respectively the dental-floss thread, is clamped and is fixed in such a manner that it is not torn out towards the free space of the U-shaped geometry, because this region explicitly serves for the application.

The application unit 12h comprises the base body 26h. The base body 26h is fixedly connected with the connection unit 18h. The base body 26h is integrally connected with the connection unit 18h. The base body 26h is connected with the connection unit 18h via an adhesive connection and/or welded connection.

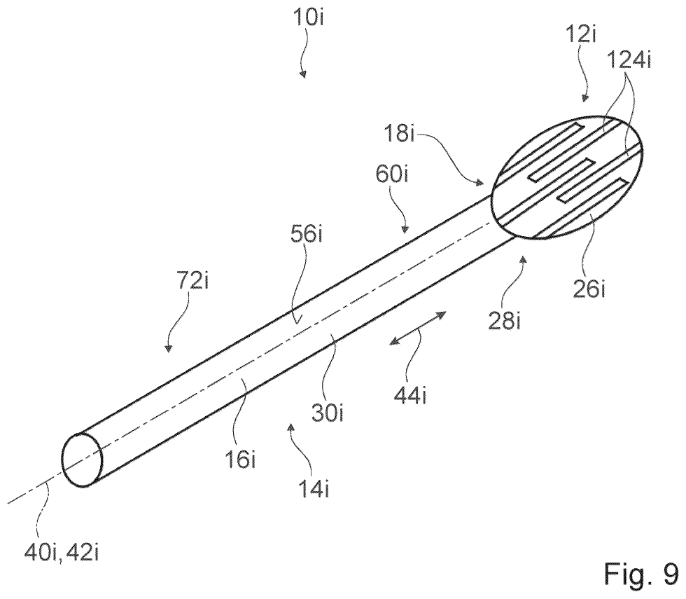

In the following, reference is made to FIG. 9, which shows a schematic 3D view of a body care product 10i according to the invention.

FIG. 9 shows a handle unit 14i with an application unit 12i of a body care product 10i in a schematic perspective illustration. The body care product 10i is realized as an applicator for a liquid or viscous medium. The body care product 10i is in the present case realized as an applicator for oral care media or medical media.

The body care product 10i comprises an application unit 12i. The body care product 10i further comprises a handle unit 14i, which has a material handle body 16i. Beyond this the body care product 10i comprises a connection unit 18i, which connects the application unit 12i to the handle unit 14i.

The body care product 10i has a longitudinal axis 42i, a height axis and a width axis. The longitudinal axis 42i is arranged parallel to a main extension direction 44i of the body care product 10i. The longitudinal axis 42i corresponds to the longitudinal axis of the handle body 16i. The height axis and the width axis are perpendicular to the longitudinal axis 42i. The width axis is arranged perpendicular to the longitudinal axis 42i and perpendicular to the height axis. In the present case the body care product 10i has a length, in particular parallel to the longitudinal axis 42i, of 60 mm to 100 mm and preferably of 75 mm to 90 mm. Due to the preferably symmetrical design of the product, the height and width dimensions (in the direction of the height axis and the width axis) are identical. The height dimension, respectively the width dimension is 2 mm to 8 mm and preferably 2 mm to 5 mm.

The application unit 12i forms an applicator head. In the present case the application unit 12i is designed as an applicator head. The application unit 12i in particular comprises a base body 26i that is realized as a body made of a soft component. The base body 26i forms a connection with the connection unit 18i. The connection unit 18i is to be understood as an element that is molded on with the applicator head.

The handle unit 14i is configured to be gripped by the operator. The handle unit 14i comprises the material handle body 16i. The material handle body 16i of the handle unit 14i comprises a head region 28i. The material handle body 16i of the handle unit 14i further comprises a handle region 72i and a neck region 60i that is arranged between the handle region 72i and the head region 28i. The head region 28i is configured to connect the application unit 12i. The handle unit 14i comprises at least one handle surface 56i, which forms a surface of the material handle body 16i.

The material handle body 16i consists at least largely of a paper material. The material handle body 16i consists entirely of a paper material. The material handle body 16i of the handle unit 14i may comprise at least one layer 30i of a paper material, which is realized in rolled fashion and thus forms the handle body 16i.

The application unit 12i is produced as a body made of a soft component. The body is designed in the manner of a sphere or an ellipsoid. Recesses, respectively depressions 124i, are formed on the surface of the body of the application unit 12i. The recesses, respectively depressions 124i, serve to temporarily store the medium to be applied. By means of the recesses, respectively depressions 124i, the medium that is to be applied is transported from a receiving location to the application location. The application unit 12i may also be flocked in order to improve the application.

The application unit 12i comprises the base body 26i. The base body 26i is fixedly connected with the connection unit 18i. The base body 26i is integrally connected with the connection unit 18i. The base body 26i is connected with the connection unit 18i by overmolding.

The structure of the body care product 10i is equipped with a number of lays and a number of layers, as mentioned in the description.

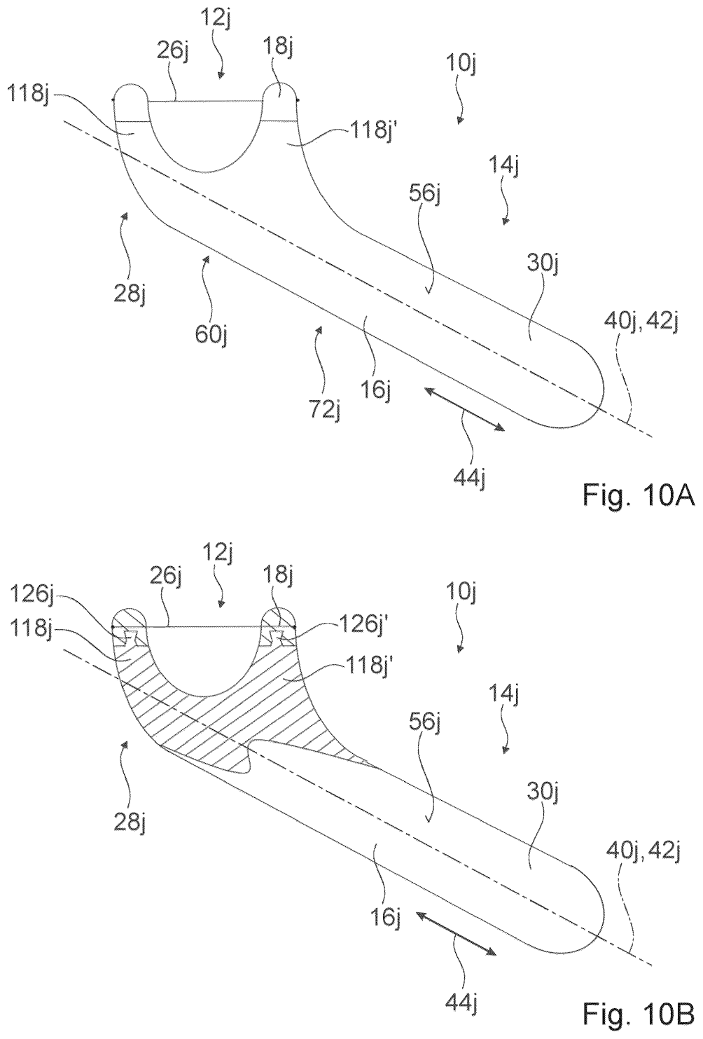

In the following, reference is made to FIGS. 10A and 10B, which show two views of the body care product 10j.

FIG. 10A schematically shows a handle unit 14j of a body care product 10j in a plan view. The body care product 10j is embodied as an oral hygiene means. The body care product 10j is in the present case realized as a flosser.

The body care product 10j comprises an application unit 12j. The body care product 10j further comprises a handle unit 14j which has a material handle body 16j. Beyond this the body care product 10j comprises a connection unit 18j which connects the application unit 12j to the handle unit 14j. The connection unit 18j is in the present case realized as an overmold body connecting the handle unit 14j to the application unit 12j.

The application unit 12j forms a dental-floss thread of the body care product 10j. In the present case the application unit 12j is realized as a dental-floss thread. The application unit 12j in particular comprises a base body 26j that is realized as a thread. The base body 26j forms a connection with the connection unit 18j. The connection unit 18j is realized as a plastic part which is molded onto the dental-floss thread and which on the one hand connects the thread to the connection unit 18j and on the other hand connects the connection unit 18j to the handle unit 14j.

The handle unit 14j is configured to be gripped by the operator. The handle unit 14j comprises the material handle body 16j. The material handle body 16j of the handle unit 14j comprises a head region 28j. The material handle body 16j of the handle unit 14j further comprises a handle region 72j and a neck region 60j that is arranged between the handle region 72j and the head region 28j. The head region 28j is realized as a U-shaped geometry with two legs, respectively arms. In a final state, the material handle body 16j has a flat basic shape which is divided into two legs 118j, 118j′ towards the application unit 12j. The handle unit 14j comprises at least one handle surface 56j, which forms a surface of the material handle body 16j.

The material handle body 16j consists at least largely of a paper material. The material handle body 16j of the handle unit 14j may comprise at least one layer 30j of a paper material. The layer may be realized in three-dimensionally formed fashion. The material handle body 16j consists of precisely one layer 30j of a paper material.

The head region 28j is designed as a U-shaped geometry with the two legs 118j, 118j′. The two legs are partially formed by the connection unit 18j, which is an overmold body. The connection unit 18j is on each of the legs 118j, 118j′ molded to the handle body 16j. The legs 118j, 118j′ are each provided with a projection 126j, 126j′, which forms an undercut and is required for the anchoring of the connection unit 18j. By way of example, the legs 118j, 118j′ each have a dovetail-shaped projection 126j, 126j′. Alternatively, the projection 126j, 126j′ which forms an undercut may be replaced by a through hole.

The anchoring of the dental-floss thread takes place in a known manner in which it can be fixed in the case of flossers made of plastic. The ends of the dental-floss thread on the outer side of the legs 118j, 118j′ may be molten/slightly burned, and thus form a spherical shape which makes pulling out of the connection unit 18j even more difficult.

In the plan view with the application region shown in section in FIG. 10B, the material connection of the connection unit 18j to the handle body 16j and to the dental-floss thread can be clearly seen. In the sectioned handle body 16j, the projection 126j, 126j′ which projects into the connection unit 18j can be seen on each of the legs 118j, 118j′. The projections 126j, 126j′ are each formed by a pin. The projections 126j, 126j′ are overmolded and provided with undercuts which support the anchoring of the connection unit 18j. It is further possible to perceive the dental-floss thread that is guided through the connection unit 18j and is molten at the ends.

The application unit 12j comprises the base body 26j. The base body 26j is fixedly connected with the connection unit 18j. The base body 26j is connected with the connection unit 18j by overmolding.

1. A body care product, in particular a razor, comprising:
with at least one application unit;
a handle unit comprising at least one material handle body; and
at least one connection unit including a form-fitting element,
wherein the at least one material handle body comprises at least for the most part of a wood, a paper material, a material made from fibers or cellulose, or combinations thereof; and
wherein the at least one connection unit is configured to attach the at least one application unit to the at least one handle unit.

2. The body care product according to claim 1,
wherein
the at least one form-fitting element is configured to engage into a recess of the application unit and/or of the handle unit.

3. The body care product according to claim 2,
wherein
the at least one material handle body delimits at least one pass-through recess which is configured to receive the at least one form-fitting element of the connection unit.

4. The body care product according to claim 2,
wherein
the at least one form-fitting element is realized as a latching element.

5. The body care product according to claim 2,
wherein
the at least one form-fitting element is realized as a rivet, in particular a plastic rivet.

6. The body care product according to claim 1,
wherein
the application unit comprises at least one base body, which is fixedly connected with the connection unit.

7. The body care product according to claim 1,
wherein
the application unit comprises at least one base body, which is exchangeably connected with the connection unit.

8. The body care product according to claim 1,
wherein
the at least one material handle body of the handle unit consists of precisely one layer of a paper material.

9. The body care product according to claim 1,
wherein
the connection unit comprises at least one receiving groove configured to receive a front edge of the at least one material handle body of the handle unit, which faces towards the application unit.

10. The body care product according to claim 1,
wherein
the at least one material handle body of the handle unit forms at least one stabilizing wall, which is angled with respect to a main extension plane of the handle unit and extends at least substantially parallel to a longitudinal axis of the handle unit.

11. The body care product according to claim 10,
wherein
the connection unit comprises at least one delimiting wall, which is configured for an at least partial delimitation and stabilization of the stabilizing wall of the at least one material handle body.

12. An application unit for a razor head, having a longitudinal axis, comprising:
a base body including a connecting unit,
wherein the connecting unit includes at least one receiving groove.

13. The application unit according to claim 12, further comprising two delimiting walls.

14. The application unit according to claim 12, wherein the at least one receiving groove is essentially perpendicular to the longitudinal axis.

15. The application unit according to claim 12, further comprising a form-fitting element.

16. A handle unit having a longitudinal axis, comprising:
a material handle body including a head region, a handle region, a front side and a rear side, the material handle body having at least one handle surface,
wherein the material handle body includes a material thickness that is substantially constant.

17. The handle unit according to claim 16, further comprising at least one recess.

18. The handle unit according to claim 16, further comprising two delimiting walls bent with respect to the remainder of the material handle body.

19. The handle unit according to claim 16, further comprising a razor blade directly attached to the handle unit.
